(19) United States
(12) Patent Application Publication
(10) Pub. No.: US 2010/0260677 A1
(43) Pub. Date: Oct. 14, 2010

(54) METHODS AND SYSTEMS FOR TREATMENT AND/OR DIAGNOSIS

(75) Inventors: Sangeeta N. Bhatia, Lexington, MA (US); Geoffrey A. von Maltzahn, Boston, MA (US)

Correspondence Address: WOLF GREENFIELD & SACKS, P.C., 600 ATLANTIC AVENUE, BOSTON, MA, 02210-2206, US

(73) Assignee: Massachusetts Institute of Technology, Cambridge, MA (US)

(21) Appl. No.: 12/716,217

(22) Filed: Mar. 2, 2010

Related U.S. Application Data
(60) Provisional application No. 61/156,676, filed on Mar. 2, 2009.

(51) Int. Cl. A61K 9/14; A61K 49/00

(52) U.S. Cl. 424/9.1; 424/489

(57) ABSTRACT

The invention relates to methods and systems that can be used for treatment and/or diagnosis. In one aspect, the present invention involves systems and methods for activating a biological cascade in a subject, and administering, to the subject, a composition or a component comprising an agent able to bind a product of a biological cascade or otherwise interact with the biological cascade. The biological cascade may be, for example, a coagulation cascade, a complement cascade, an inflammation cascade, or the like. In some cases, the concentration of a protein, the metabolic demand for a substrate, or the like may be increased as a result of activation of the biological cascade. As a specific non-limiting example, in one set of embodiments, the biological cascade may be a coagulation cascade and the composition administered to the subject may include a fibrin-binding peptide and an antitumor species. By activating the biological cascade, e.g., with an activation composition or by applying energy, coagulation may be induced in a tumor, which the antitumor species may associate with due to an increase in fibrin caused by the coagulation cascade. In addition, in certain aspects, the present invention involves systems and methods for changing tissue from a first state to a second state, for instance, with a first composition comprising nanoparticles. The second composition may be more responsive to the tissue in the second state than in the first state in some cases. In still other aspects, the present invention is generally directed to systems and methods for making such compositions, systems and methods for promoting such compositions, kits involving such compositions, or the like.

### RELATED APPLICATIONS

This application claims the benefit of U.S. Provisional Patent Application Ser. No. 61/156,676, filed Mar. 2, 2009, entitled “Methods and Systems for Treatment and/or Diagnosis,” by von Maltzahn, et al., incorporated herein by reference.

### FIELD OF INVENTION

The invention relates to methods and systems that can be used for treatment and/or diagnosis.

### BACKGROUND

Targeted drug delivery is a method of delivering medication to a subject in a manner that increases the concentration of the medication in some parts of the body relative to others. Many systems have been used for targeted drug delivery, including antibodies and nanoparticles. However, the increase in concentration in a particular location has not been great in existing approaches, and alternative approaches for targeted drug delivery are thus still needed.

### SUMMARY OF THE INVENTION

The invention relates to methods and systems that can be used for treatment and/or diagnosis. The subject matter of the present invention involves, in some cases, interrelated products, alternative solutions to a particular problem, and/or a plurality of different uses of one or more systems and/or articles.

In one set of embodiments, the present invention is directed to a method includes acts of administering, to a subject, a receiving composition comprising an agent able to bind a product of a biological cascade, and prior to, simultaneously with, and/or after the act of administering, activating the biological cascade in the subject, thereby facilitating association of the receiving composition with the biological cascade. The present invention, in another set of embodiments, is directed to a method comprising increasing a concentration of a protein in a selected site by activating a biological cascade within the target tissue, and exposing the target tissue to a composition comprising an agent able to bind the protein.

In another set of embodiments, is directed to a method comprising increasing metabolic demand for a substrate of a biological cascade in a target tissue, and exposing the target tissue to a composition comprising an agent able to bind a product of a biological cascade produced from the substrate. In still another set of embodiments, the present invention is directed to a method comprising administering, to a subject, a composition comprising a fibrin-binding peptide and an antitumor composition, and prior to, simultaneously with, and/or after the act of administering, activating coagulation within a tumor within the subject, thereby causing the composition to bind proximate the tumor. The present invention, in another set of embodiments, is directed to a method comprising administering, to a subject, a composition comprising a substrate to which activated Factor XIII specifically binds and an antitumor agent, and prior to, simultaneously with, and/or after the act of administering, activating coagulation within a tumor within the subject, thereby causing the composition to bind proximate the tumor.

In another set of embodiments, the invention features a method including changing tissue from a first state to a second state with a first composition having nanoparticles, and delivering a second composition to the tissue. In some cases, the second composition is more responsive to the tissue in the second state than in the first state. The second composition can include nanoparticles having a composition different from the composition of the nanoparticles of the first composition.

In another set of embodiments, the invention features a method includes coagulating a portion of tissue, and delivering a first composition to the coagulated tissue. In some cases, the first composition is more responsive to the coagulated tissue than un-coagulated tissue. The invention, in yet another set of embodiments, includes a method of inflaming a portion of tissue, and delivering a first composition to the coagulated tissue. The first composition, in some cases, is more responsive to the inflamed tissue than non-inflamed tissue. In yet another set of embodiments, the invention features a method including increasing an amount of selected binding sites at a portion of tissue, and introducing a first composition to the tissue. In some embodiments, the first composition is adapted to bind to the binding sites.

In one set of embodiments, the invention is directed to a system including a first composition capable of changing tissue from a first state to a second state, and a second composition adapted to be more responsive to the tissue in the second state than in the first state. The first composition may have nanoparticles in some cases. The invention features a system including a tissue coagulant, according to another set of embodiments. In some cases, the first composition may be adapted to be more responsive to coagulated tissue than un-coagulated tissue. In another set of embodiments, the invention features a system including a tissue inflammatory material, and a first composition adapted to be more responsive to inflamed tissue than non-inflamed tissue. The invention, in still another set of embodiments, includes a system comprising a first composition capable of increasing an amount of selected binding sites at a portion of tissue, and a second composition adapted to bind to the binding sites.

In one set of embodiments, the present invention is directed to an article, comprising a receiving composition comprising an agent able to bind a product of a biological cascade, and an activation composition able to activate the biological cascade. In some cases, the receiving composition is distinguishable from the activation composition. In another set of embodiments, the present invention includes an article comprising a receiving composition comprising an agent able to bind a product of an inflammation cascade, and an activation composition able to activate the inflammation cascade. In yet another set of embodiments, the present invention is directed to an article comprising a receiving composition comprising an agent able to bind a product of a complement cascade, and an activation composition able to activate the complement cascade.

In one set of embodiments, the present invention is directed to a kit comprising a receiving composition comprising an agent able to bind a product of a biological cascade, and an activation composition able to activate the biological cascade. The present invention, in another set of embodiments, includes a kit comprising a receiving composition comprising an agent able to bind a product of a biological cascade, and an applicator for applying energy to a subject. In yet another set of embodiments, the present invention is directed to a kit comprising a receiving composition comprising an agent able to bind a product of a biological cascade, and an activation composition able to activate the biological cascade.

Several methods are disclosed herein of administering a subject with a compound for prevention or treatment of a particular condition. It is to be understood that in each such aspect of the invention, the invention specifically includes, also, the compound for use in the treatment or prevention of that particular condition, as well as use of the compound for the manufacture of a medicament for the treatment or prevention of that particular condition.

In another aspect, the present invention is directed to a method of making one or more of the embodiments described herein. In another aspect, the present invention is directed to a method of using one or more of the embodiments described herein.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIGS. 1A-1B are schematic diagrams of various embodiments of methods of treatment and/or diagnosis;

FIGS. 2A-2C show nanoparticle communication for amplified tumor targeting, in accordance with certain embodiments of invention;

FIGS. 3A-3H show various signaling components, in other embodiments of the invention;

FIGS. 4A-4F show various receiving components, in other embodiments of the invention;

FIGS. 5A-5J illustrate amplified tumor targeting, in one set of embodiments;

FIGS. 6A-6E illustrate amplified tumor therapy, in another sets of embodiments;

FIGS. 7A-7C illustrate specific fibrinogen tropism to heated tumors, in one set of embodiments;

FIGS. 8A-8E illustrate nanorod-directed tumor heating and fibrinogen deposition, in another set of embodiments;

FIGS. 9A-9C illustrate tTF-RGD and tTF-NGR signaling components, in accordance with yet another set of embodiments;

FIGS. 10A-10D illustrate absorption, fluorescence, and hydrodynamic size characterization of various receiving components, in still another set of embodiments;

FIGS. 11A-11F show various nanoworm receivers, in yet another set of embodiments;

FIG. 12 shows data from cytotoxicity experiments to assess intrinsic toxicity of Au nanorods and doxorobicin-loaded liposomes, in certain embodiments of the invention;

FIGS. 13A-13B illustrate integrated communicating nanoparticles in certain embodiments of the invention;

FIG. 14 shows various histopathological analyses of FXIII-targeted nanoworms, in another set of embodiments;

FIG. 15 shows nanoparticle communication in human cervical xenograft tumors, in still another set of embodiments;

FIGS. 16A-16E illustrate autonomous communication between tTF-RGD signaling components and FXIII-NW receivers, in yet another set of embodiments;

FIGS. 17A-17B illustrate certain therapeutic communicating nanoparticles in one set of embodiments;

FIGS. 18A-18C show small molecule vascular disrupting agents to selectively induce coagulation in tumors, in another set of embodiments; and

FIG. 19 shows the upregulation of ICAM-1 in response to certain growth factors and cytokines, in another embodiment of the invention.

### BRIEF DESCRIPTION OF THE SEQUENCES

SEQ ID NO: 1 is G-Y-e-C-hyP-cY-G-L-C-Y-I-Q, a fibrin binding peptide, wherein e is D-glutamic acid, hyP is hydroxyproline, and cY is chlorotyrosine;

SEQ ID NO: 2 is a peptide recognizable by Factor XIII having a sequence GNQEQVSPLTLLKXC, where X is a 6-aminohexanoic acid;

SEQ ID NO: 3 is Ac-d-d-d-G-Y-e-C-hyP-cY-G-L-C-Y-I-Q-K-Fl, a fibrin binding peptide, where Ac is the acetyl end terminus, d is D-aspartic acid, e is D-glutamic acid, hyP is hydroxproline, and cY is chlorotyrosine;

SEQ ID NO: 4 is KK, a target sequence for Cathepsin B;

SEQ ID NO: 5 is HSSKLQ, a target sequence for PSA;

SEQ ID NO: 6 is PICFF, a target sequence for Cathepsin D;

SEQ ID NO: 7 is GPLGVRG, a target sequence for MMP-2

SEQ ID NO: 8 is GVSQNYPIVG, a target sequence for HIV protease;

SEQ ID NO: 9 is LVLASSSFGY, a target sequence for HSV protease;

SEQ ID NO: 10 is DEVD, a target sequence for Caspase-3;

SEQ ID NO: 11 is WEHD, a target sequence for Caspase-1;

SEQ ID NO: 12 is G-N-A-E-Q-V-S-P-L-T-L-L-K-X-C-(K-Fluorescein), a control substrate for Factor XIII, where X is a 6-aminohexanoic acid;

SEQ ID NO: 13 is GLPCDYYGTCLD, a fibrin-binding peptide;

SEQ ID NO: 14 is GYLCGDYTLCPD, a fibrin-binding peptide;

SEQ ID NO: 15 is LPCDYYGTC-Bip, a fibrin-binding peptide, where Bip is L-biphenylalanine;

SEQ ID NO: 16 is LPCDYYGTC-Bip-d, a fibrin-binding peptide, where Bip is L-biphenylalanine and d is the D-isomer of aspartic acid;

SEQ ID NO: 17 is CGLIIQKNEC, a fibronectin-targeting ligand;

SEQ ID NO: 18 is CNAGESSKNC, a fibronectin-targeting ligand;

SEQ ID NO: 19 is a linear alpha-v-beta-3 targeting ligand matching the targeting motif on tTF-RGD, fluorescein-X-C-G-R-G-D-S-P-COO-, where X is a 6-aminohexanoic acid;

SEQ ID NO: 20 is a CD13/aminopeptidase N targeting ligand matching the motif on tTF-NGR, Fluorescein-X-C-G-N-G-R-A-H-A-COO-, where X is a 6-aminohexanoic acid;

SEQ ID NO: 21 is the primer CATGCCATGGGATCAGGCACTACAAATACTGTGGCAGCATATAAT;

SEQ ID NO: 23 is the primer CGGGATCCTATTATGGAGAATCACCTCTTCCTCTGAATTCCCC; and

SEQ ID NO: 24 is the primer CGGGATCCTATTATGCATGTGCTCTTCCGTTACCTCTGAATTCCCC.

### DETAILED DESCRIPTION

The invention relates to methods and systems that can be used for treatment and/or diagnosis. In one aspect, the present invention involves systems and methods for activating a biological cascade in a subject, and administering, to the subject, a composition or a component comprising an agent able to bind a product of a biological cascade or otherwise interact with the biological cascade. The biological cascade may be, for example, a coagulation cascade, a complement cascade, an inflammation cascade, or the like. In some cases, the concentration of a protein, the metabolic demand for a substrate, or the like may be increased as a result of activation of the biological cascade. As a specific non-limiting example, in one set of embodiments, the biological cascade may be a coagulation cascade and the composition administered to the subject may include a fibrin-binding peptide and an antitumor species. By activating the biological cascade, e.g., with an activation composition or by applying energy, coagulation may be induced in a tumor, which the antitumor species may associate with due to an increase in fibrin caused by the coagulation cascade. In addition, in certain aspects, the present invention involves systems and methods for changing tissue from a first state to a second state, for instance, with a first composition comprising nanoparticles. The second composition may be more responsive to the tissue in the second state than in the first state in some cases. In still other aspects, the present invention is generally directed to systems and methods for making such compositions, systems and methods for promoting such compositions, kits involving such compositions, or the like.

A first aspect of the invention is generally directed to systems and methods for using biological cascades to enhance and/or localize delivery of certain compositions or components, e.g., to a selected site within a subject. For example, one set of embodiments is generally directed to systems and methods for administering, to a subject, a composition comprising an agent able to bind a product of a biological cascade, or otherwise interact with the biological cascade, and activating the biological cascade in the subject, which may facilitate association of the composition with the biological cascade. These can be done in any order, including simultaneously.

Biological cascades are naturally-occurring series of chemical reactions in which the products of one reaction are consumed in the next reaction. Often, such biological cascades provide an enhancement in signal, since a product produced in one step may be able to catalyze the formation of multiple products in the next step within the biological cascade. Thus, activating a biological cascade with a relatively small initial signal may result in the production of a relatively large response by the biological cascade. In some cases, activation of a biological cascade may increases the concentration of a protein and/or the metabolic demand for an agent within the biological cascade. Biological cascades may be identified as a series of reactions in which the product of one reaction catalyzes the next reaction. In contrast, in many other metabolic processes (e.g., glucose oxidation), the product of one reaction is used as the reactant for the next reaction, not the catalyst that facilitates the next reaction. As discussed herein, certain embodiments of the invention are directed to using biological cascades or similar processes to facilitate or enhance and/or localize the binding of compositions or compositions within a subject, e.g., to selected sites within the subject. In some embodiments, the biological cascade may be a cascade that occurs extracellularly of cells, e.g., as in the coagulation cascade or the inflammation cascade.

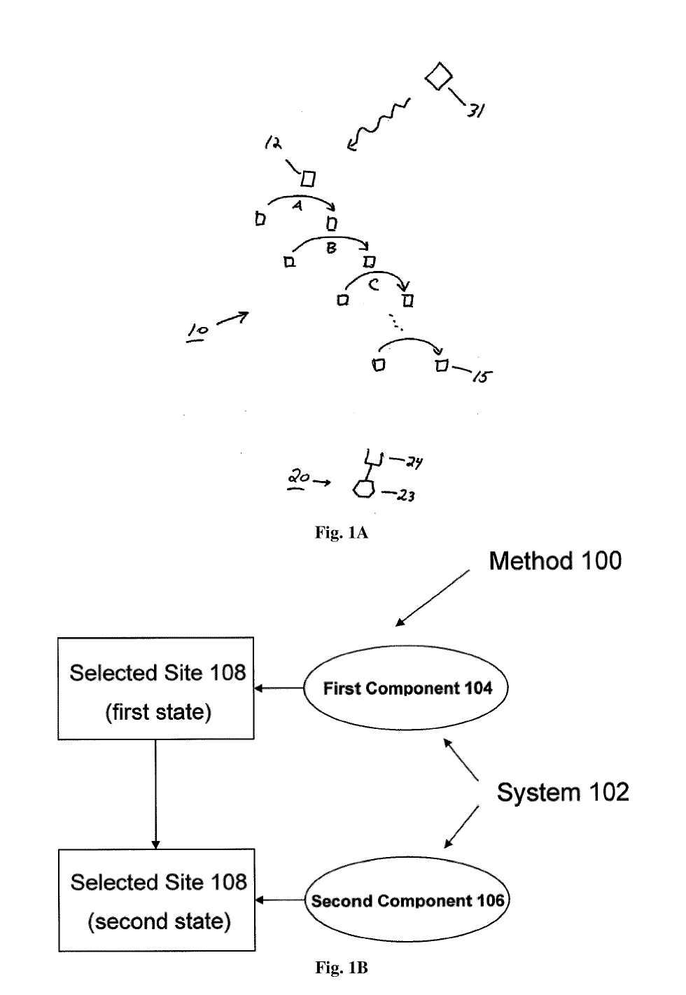

Referring now to the schematic drawing shown in the example of FIG. 1A with biological cascade 10, a series of reactions is shown that occurs between a first reactant 12 and a final product 15. In this figure, first reactant 12 catalyzes Reaction A, which in turn produces a product (e.g., an activated enzyme) catalyzing Reaction B, etc., until final product 15 is produced from the biological cascade. Composition 20 may also be administered, where composition 20 includes an agent 24 able to bind or interact with final product 15 in some fashion. Composition 20 may also include other agents 23, such as a pharmaceutical agent or a diagnostic agent (or other agents such as those described herein), for example, for therapeutic or diagnostic purposes. Biological cascade 10 may be activated by any suitable technique, for example, by supplying an activation composition 31 which is able to activate biological cascade 10, and/or by supplying energy to activate the biological cascade 10.

As a specific non-limiting example of a biological cascade, the coagulation cascade is a biological cascade that directs blood clotting. In this cascade, a series of reactions occurs which eventually leads to the production of fibrin, a fibrous protein that is polymerized to form a “mesh” over a wound site to form a blood clot. By administering, to a subject, a receiving composition comprising a suitable agent able to bind a product of a biological cascade, or otherwise interact with the biological cascade, for example, a fibrin-binding peptide or a peptide substrate for Factor XIII, and activating coagulation within a selected site within the subject, the composition may be targeted to the selected site. As a specific non-limiting example, the receiving composition includes an antitumor species in some embodiments; coagulation can be activated within or proximate a tumor within a subject, thereby causing fibrin to form within or proximate the tumor. The receiving composition may thus be able to bind fibrin due to the presence of a suitable agent within the receiving composition, such as a fibrin-binding peptide. Optionally, the receiving composition may also include particles, nanoworms, liposomes, etc., which may facilitate administration of the composition. Once bound to the fibrin, the antitumor species may be positioned in, or at least proximate, the tumor, which may thus be used to treat the tumor. As discussed herein, coagulation within the tumor may be activated using any suitable technique, including chemical techniques (e.g., an activation composition), physical techniques, energy, etc.

Another example schematic drawing is shown with reference to FIG. 1B. This figure shows an example method 100 for medical treatment and/or diagnosis. As shown in this example, method 100 includes system 102 that includes first component 104 and second component 106. First component 104 and second component 106 may work together to provide treatment and/or diagnosis to a subject. For example, first component 104 may be capable of directly or indirectly changing a selected site 108 (e.g., a tumor) from a first state to a second state. Second component 106 may include, for instance, a pharmaceutical agent and/or a diagnostic agent, for example, a drug and/or an imaging agent. Furthermore, second component 106 in this example is adapted to target or to recognize selected site 108 when the selected site is in the second state. By changing selected site 108 with first component 104 to a second state that can be targeted by second component 106, delivery of the second component to the selected site for treatment and/or diagnosis can be enhanced. In comparison, when selected site 108 is in the first state, relatively less of second component 106 may be delivered to the selected site (for example, second component 106 may associate with the selected site less strongly or not at all beyond background interactions in some cases). As a non-limiting example, first component 104 can include a material capable of coagulating within a tumor, and second component 106 can include a material capable of targeting coagulated tumor and delivering a tumor-destroying drug or an antitumor species to the tumor. By changing the tumor from un-coagulated tissue (the first state) to coagulated tissue (the second state), more tumor-destroying drug can be selectively delivered to the tumor, relative to delivering the drug without such a targeting functionality.

The biological cascade may be activated using any suitable technique. As used herein, “activation” includes not only activating a cascade when it was not formerly activated (e.g., activating the coagulation cascade in a tumor or other selected site), but also upregulating a previously active cascade or increasing its throughput in some cases. For example, an inflammation cascade may be activated to exhibit a greater degree of inflammation or inflammation response.

Referring again to FIG. 1A, an example of a technique for activating a biological cascade is now discussed as a non-limiting example. In this figure, biological cascade 10 is activated using activation composition 31. Activation composition 31 may be a composition that directly interacts with a portion of the biological cascade (for example, as a substrate of an enzyme in the biological cascade, as a mimic of a component of the biological cascade, as a substrate or substrate analog for reacting with the biological cascade, etc.), and/or activation composition 31 may be a composition that indirectly activates the biological cascade. In some cases, activation composition 31 may include more than one species, and/or activation composition 31 may itself be activated or triggered using an external signal, for example, by applying energy to the activation composition.

Referring again to the coagulation cascade as a specific non-limiting example, the coagulation cascade is activated, in certain embodiments, by physically causing injury to a selected site within a subject. For example, physical injury may be caused by applying pressure to the selected site, physically cutting or abrading the selected site (e.g., with a probe, a scalpel, a blade, etc.), applying heat or energy to the selected site, or the like. As another example, a chemical may be used to cause an injury to the selected site, e.g., by applying an acid or other reactive chemical to the site that causes internal bleeding.

Coagulation may be activated in a selected site by using a “signaling” or an activation composition that is able to target the selected site, according to certain embodiments of the invention. For example, a tumor within a subject can be targeted, e.g., actively using certain targeting entities such as antibodies or peptides such as RGD or NGR peptides, and/or passively using particles such as nanorods. For instance, particles such as nanorods may passively target to tumors due to the presence of fenestrated angiogenic blood vessels typically found in tumors, which are relatively small openings within the blood vessels which allow for the rapid exchange of metabolites between the blood and the tumor. Often there may be an increase in fenestrae near tumors, e.g., due to angiogenesis, which may facilitate tumor growth. Particles such as nanorods are unable able to freely pass through such fenestrae, and accordingly may accumulate there. Thus, in some embodiments, gold nanorods may be passively targeted to tumors, and near-infrared energy applied to the tumor; the near-infrared energy heats the gold nanorods, and this heat may damage the tumor, which can activate the coagulation cascade within the tumor. In contrast, near-infrared energy does not heat tissue not having gold nanorods present to the same degree as with the gold nanorods present; thus, tumors may be particularly affected by the application of near-infrared energy, relative to the surrounding non-tumor containing tissues. Accordingly, in certain embodiments, the activation composition by itself may not activate coagulation at the selected site, and coagulation instead may be separately activated at the selected site.

As a specific non-limiting example, a coagulation cascade may be activated by administering gold particles coated with poly(ethylene glycol) to a subject, then applying near infrared light to the tumors to cause heat damage and thereby activate coagulation. It should also be noted that for some applications, such as tumor treatment, the use of inherent biological cascades found within the a subject, such as the coagulation cascade or the inflammatory cascade, may potentially delay or prevent the development of inherent resistance to such treatments (for example, resistance by a tumor to continued treatments).

As another example, the activation composition may include truncated human tissue factor protein (tTF) that is activated upon binding of tTF to target receptors in tumor blood vessels. Binding of such proteins may be used to trigger coagulation, which in turn activates the coagulation cascade, thereby facilitating subsequent binding or association with receiving compositions such as those discussed herein.

As the above non-limiting examples indicate, various embodiments of the invention are generally directed to various methods and systems that can be used for treatment and/or diagnosis. It should be understood, however, that the above examples are not intended to be limiting, and additional details and further embodiments of the invention will be discussed in more detail below.

Accordingly, certain aspects of the invention includes a receiving composition or an actuator composition comprising an agent able to bind a product of a biological cascade, or an agent that is otherwise able to interact with the biological cascade, and/or a modulating entity. The composition may be any suitable composition, and may include more than agent, which may be covalently bound together or otherwise associated with each other, e.g., through ionic bonds, van der Waals forces, or the like. For example, the composition may include two or more agents that are conjugated, linked, attached, tethered, etc. The agents may be the same or different. The agents can be delivered to a selected site within a subject individually, or in combination with other agents. In some embodiments, the receiving composition may include particles (e.g., nanoparticles), microparticles, dendrimers, micelles, liposomes, or the like. For example, the composition may include a particle or a liposome to which one or more agents are associated (e.g., bound to), e.g., as described below.

In certain embodiments, the receiving composition may include a first agent able to bind a product of a biological cascade, and/or a second agent such as a pharmaceutical agent, a diagnostic agent, or the like. In some cases, the composition may include a targeting entity, e.g., as discussed below. For example, a composition of the invention comprising a diagnostic agent may be administered to a subject, optionally targeted to a selected site within the subject (e.g., to an organ, to an infection site, to a tumor, etc.), and the diagnostic agent determined or imaged in some fashion. Similarly, a composition of the invention comprising a pharmaceutical agent may be administered to a subject and optionally targeted to a selected site within a subject. As yet another example, as described herein, the changes at a site (e.g., site 108 in FIG. 1B) from a first state to a second state may provide enhanced delivery of the receiving component (e.g., component 106 in FIG. 1B); the component can include any material capable of recognizing or targeting (e.g., binding) a selected site in its second state.

In accordance with one set of embodiments, the biological cascade may be the coagulation cascade. In some cases, the coagulation cascade may be activated as discussed herein, and a receiving composition able to interact with the coagulation cascade, e.g., by binding a product of the coagulation cascade is used. Activation may be global or local, e.g., at a selected site. Upon activation, increased or enhanced binding of the receiving composition may result, e.g., at the selected site. The coagulation cascade has at least two primary pathways, the contact activation pathway (or the intrinsic pathway), and the tissue factor pathway (or the extrinsic pathway), which lead to fibrin formation. Coagulation factors are generally indicated by an “F” or the word “Factor” followed by Roman numerals, with a lowercase “a” appended to indicate an active form of the coagulation factor. The coagulation factors are all generally serine proteases (i.e., enzymes). There are some exceptions. For example, FVIII and FV are glycoproteins, and Factor XIII is a transglutaminase. Serine proteases generally act by cleaving other proteins at specific sites. The coagulation factors circulate as inactive zymogens, prior to being activated. The coagulation cascade may be divided into three pathways, including the tissue factor and the contact activation pathways, both of which can activate the “final common pathway” of Factor X, thrombin and fibrin, thereby causing blood coagulation or clotting to occur.

The tissue factor pathway can generate a so-called “thrombin burst,” which is a process by which thrombin is released essentially instantaneously. FVIIa circulates in a higher amount than other activated coagulation factors. Thus, following damage to a blood vessel, FVII leaves the circulation and comes into contact with tissue factor (TF) expressed on tissue-factor-bearing cells (stromal fibroblasts and leukocytes), forming an activated complex (TF-FVIIa). TF-FVIIa activates FIX and FX. FVII is itself activated by thrombin, FXIa, plasmin, FXII and FXa. The activation of FXa by TF-FVIIa is almost immediately inhibited by tissue factor pathway inhibitor (TFPI). FXa and its co-factor FVa form the prothrombinase complex, which activates prothrombin to thrombin. Thrombin then activates other components of the coagulation cascade, including FV and FVIII (which activates FXI, which, in turn, activates FIX), and activates and releases FVIII from being bound to vWF (von Willebrand factor). FVIIIa is the co-factor of FIXa, and together they form the “tenase” complex, which activates FX; and so the cycle continues.

The contact activation pathway begins with formation of the primary complex on collagen by high-molecular-weight kininogen (HMWK), prekallikrein, and FXII (Hageman factor). Prekallikrein is converted to kallikrein and FXII becomes FXIIa. FXIIa converts FXI into FXIa. Factor XIa activates FIX, which with its co-factor FVIIIa form the tenase complex, which activates FX to FXa.

Thrombin has a variety of functions. One role of thrombin is the conversion of fibrinogen to fibrin, which is a protein that acts as the building block of a hemostatic plug or blood clot. In addition, thrombin activates Factors VIII and V and their inhibitor protein C (in the presence of thrombomodulin), and it activates Factor XIII, which forms covalent bonds that crosslink the fibrin polymers that form from activated monomers. Following activation by the contact factor or tissue factor pathways, the coagulation cascade may be maintained in a prothrombotic state by the continued activation of FVIII and FIX to form the tenase complex, until it is down-regulated by anticoagulant pathways.

In some cases, platelet activation can occur as part of the coagulation cascade. Damage to blood vessel walls exposes subendothelium proteins, most notably von Willebrand factor (vWF), present under the endothelium. vWF is a protein secreted by healthy endothelium, forming a layer between the endothelium and underlying basement membrane. When the endothelium is damaged, the normally-isolated, underlying vWF is exposed to blood and recruits Factor VIII, collagen, and other clotting factors. Circulating platelets bind to collagen with surface collagen-specific glycoprotein Ia/IIa receptors. This adhesion is strengthened further by additional circulating proteins vWF, which forms additional links between the platelets glycoprotein Ib/IX/V and the collagen fibrils. These adhesions activate the platelets.

Activated platelets may release the contents of stored granules into the blood plasma. The granules include ADP, serotonin, platelet-activating factor (PAF), vWF, platelet factor 4, and thromboxane A2 (TXA2), which, in turn, activate additional platelets. The granules' contents activate a Gq-linked protein receptor cascade, resulting in increased calcium concentration in the platelets' cytosol. The calcium activates protein kinase C, which, in turn, activates phospholipase A2 (PLA2). PLA2 then modifies the integrin membrane glycoprotein IIb/IIIa, increasing its affinity to bind fibrinogen. The activated platelets change shape from spherical to stellate, and the fibrinogen cross-links with glycoprotein IIb/IIIa aid in aggregation of adjacent platelets.

Another pathway is the contact activation pathway (intrinsic). The contact activation pathway begins with formation of the primary complex on collagen by high-molecular-weight kininogen (HMWK), prekallikrein, and FXII (Hageman factor). Prekallikrein is converted to kallikrein and FXII becomes FXIIa. FXIIa converts FXI into FXIa. Factor XIa activates FIX, which with its co-factor FVIIIa form the tenase complex, which activates FX to FXa. The minor role that the contact activation pathway has in initiating clot formation can be illustrated by the fact that patients with severe deficiencies of FXII, HMWK, and prekallikrein do not have a bleeding disorder.

The final common pathway is as follows. Thrombin has a large array of functions. Its primary role is the conversion of fibrinogen to fibrin, the building block of a hemostatic plug. In addition, it activates Factors VIII and V and their inhibitor protein C (in the presence of thrombomodulin), and it activates Factor XIII, which forms covalent bonds that crosslink the fibrin polymers that form from activated monomers. Following activation by the contact factor or tissue factor pathways, the coagulation cascade is maintained in a prothrombotic state by the continued activation of FVIII and FIX to form the tenase complex, until it is down-regulated by the anticoagulant pathways.

There are several mechanisms keep platelet activation and the coagulation cascade in check, including the following. Any of these may be targeted to control the coagulation cascade.

Protein C is a major physiological anticoagulant. It is a vitamin K-dependent serine protease enzyme that is activated by thrombin into activated protein C (APC). Protein C is activated in a sequence that starts with Protein C and thrombin binding to a cell surface protein thrombomodulin. Thrombomodulin binds these proteins in such a way that it activates Protein C. The activated form, along with protein S and a phospholipid as cofactors, degrades FVa and FVIIIa. Quantitative or qualitative deficiency of either may lead to thrombophilia (a tendency to develop thrombosis). Impaired action of Protein C (activated Protein C resistance), for example by having the “Leiden” variant of Factor V or high levels of FVIII also may lead to a thrombotic tendency.

Antithrombin is a serine protease inhibitor (serpin) that degrades the serine proteases: thrombin, FIXa, FXa, FXIa, and FXIIa. It is constantly active, but its adhesion to these factors is increased by the presence of heparan sulfate (a glycosaminoglycan) or the administration of heparins (different heparinoids increase affinity to FXa, thrombin, or both). Quantitative or qualitative deficiency of antithrombin (inborn or acquired, e.g., in proteinuria) leads to thrombophilia.

Tfactor pathway inhibitor (TFPI) limits the action of tissue factor (TF). It also inhibits excessive TF-mediated activation of FIX and FX.

Plasmin is generated by proteolytic cleavage of plasminogen, a plasma protein synthesized in the liver. This cleavage is catalyzed by tissue plasminogen activator (t-PA), which is synthesized and secreted by endothelium. Plasmin proteolytically cleaves fibrin into fibrin degradation products that inhibit excessive fibrin formation.

Prostacyclin (PGI2) is released by endothelium and activates platelet Gs protein-linked receptors. This, in turn, activates adenylyl cyclase, which synthesizes cAMP. cAMP inhibits platelet activation by decreasing cytosolic levels of calcium and, by doing so, inhibits the release of granules that would lead to activation of additional platelets and the coagulation cascade.

It should also be noted that, as discussed in detail below, the coagulation cascade can be activated using multiple techniques, including but not limited to the activation of any of the enzymes or factors discussed above. Other methods of activating the coagulation cascade are also discussed below.

In another set of embodiments, the biological cascade may be the complement cascade or the complement system. The complement system is a biological cascade that helps, or “complements,” the ability of antibodies to clear pathogens from a subject. It is part of the immune system called the innate immune system. However, it can be recruited and brought into action in some cases by the adaptive immune system. The complement system includes various relatively small proteins found in the blood, generally synthesized by the liver, and normally circulating as inactive precursors (pro-proteins). When stimulated by one of several triggers, proteases in the system cleave specific proteins to release cytokines and initiate an amplifying cascade of further cleavages. One result of this activation cascade is massive amplification of a response, including activation of the cell-killing membrane attack complex. Over 25 proteins and protein fragments make up the complement system, including serum proteins, serosal proteins, and cell membrane receptors. These proteins are synthesized mainly in the liver, and they account for about 5% of the globulin fraction of blood serum. Various biochemical pathways activate the complement system, including the classical complement pathway and the alternative complement pathway.

The classical pathway of activation of the complement system is a group of blood proteins that mediate the specific antibody response. It is triggered by antigen-bound antibody molecules. It is the binding of a specific part of the antibody molecule to the C1 component that initiates this pathway. The initial enzyme, C1, is a complex formed through a calcium-dependent association between two reversibly interacting subunits, C1q and C1 (C1qr2s2). Approximately 70% of C1 is typically present in this complex form. C1 occurs in serum as a proenzyme which tends to undergo autoactivation but which is controlled by C1-inhibitor (e.g., C1-in or C1 esterase). Upon the binding of C1 to immune complexes by virtue of the affinity of C1q for immunoglobulins (e.g., IgM and IgG), the controlling action of C1-In may be overcome and C1q causes activation of C1r2s2.

C1q appears to possess substantially no intrinsic catalytic activity, but when any of several activators bind to the C1q subcomponent of C1, the homologous C1r and C1s subcomponents are converted into catalytically active species, namely C1r* and C1s*, thereby activating the classical pathway of complement activation. Thus, on binding to immune complexes through C1q, the subunits of C1 become firmly associated and autoactivation commences. Initially, a conformational change in C1r occurs, followed by proteolytic activation which results in the cleavage of all four polypeptide chains of C1r2s2. The two activated C1s subunits are then able to catalyze the assembly of the C3-convertase, C4bC2a, which has been formed from C2 and C4.

The alternative pathway of the complement system is an innate component of the immune system's natural defense against infections which can generally operate without antibody participation. The alternative pathway opsonizes and kills pathogens. The alternative pathway does not necessarily require a specific antibody to commence, and so it can usually be effective much faster than if antibody synthesis had to take place, as in the classical pathway. However, not all antigens can activate this pathway.

The alternative pathway may be initiated by the spontaneous hydrolysis of C3, which is abundant in the plasma in the blood. “Tickover” occurs through the spontaneous cleavage of the thioester bond in C3 to form C3(H₂O). This change in shape allows the binding of plasma protein Factor B which allows Factor D to cleave Factor B into Ba and Bb. Bb remains part of the C3(H₂O) to form C3(H₂O)Bb. This complex is also known as a fluid-phase C3 convertase. This convertase, although produced in small amounts, can cleave C3 proteins into C3a and C3b. C3a stimulates histamine release by mast cells, thereby producing vasodilation. C3b is able to bind to bacterial cell walls and act as an opsonin, which marks the invader as a target for phagocytosis. The alternative pathway C3-convertase includes the activated B and D factors, forming a generally unstable compound. This compound can become stable under certain conditions, e.g., after binding properdin, a serum protein. After the creation of C3 convertase, the complement system follows the same path regardless of activation, as discussed above. Binding of another C3b-fragment to the C3-convertase of the alternative pathway may create a C5-convertase analogous to the MBL or classical pathway. C5 stimulates histamine release by mast cells, thereby producing vasodilation. It is also able to act as a chemoattractant to direct cells via chemotaxis to the site of inflammation.

As mentioned, the complement cascade includes the classical pathway, the alternative pathway, and the lectin pathway. The classical pathway may be activated by activation of the C1-complex (C1q, two molecules of C1r, and two molecules of C1s thus forming C1qr2s2), which occurs when C1q binds to IgM or IgG complexed with antigens (a single IgM can initiate the pathway, while multiple IgGs are needed), or when C1q binds directly to the surface of the pathogen. Such binding leads to conformational changes in the C1q molecule, which leads to the activation of two C1r (a serine protease) molecules. They then cleave C1s (another serine protease). The C1r2s2 component now splits C4 and then C2, producing C4a, C4b, C2a, and C2b. C4b and C2a bind to form the classical pathway C3-convertase (C4b2a complex), which promotes cleavage of C3 into C3a and C3b; C3b later joins with C4b2a (the C3 convertase) to make C5 convertase (C4b2a3b complex). The inhibition of C1r and C1s is controlled by C1-inhibitor. C3-convertase can be inhibited by decay accelerating factor (DAF), which is bound to erythrocyte plasma membranes via a GPI anchor.

The alternative pathway may be activated by spontaneous C3 hydrolysis directly due to the breakdown of the thioester bond via condensation reaction (C3 is mildly unstable in aqueous environment) to form C3a and C3b. It does not rely on a pathogen-binding antibodies like the other pathways. C3b is then capable of covalently binding to a pathogenic membrane surface if it is near enough. If there is no pathogen in the blood, the C3a and C3b protein fragments may be deactivated by rejoining with each other. Upon binding with a cellular membrane C3b is bound by factor B to form C3bB. This complex in presence of factor D will be cleaved into Ba and Bb. Bb will remain covalently bonded to C3b to form C3bBb which is the alternative pathway C3-convertase. The protein C3 maybe produced in the liver.

The C3bBb complex, attaches to the surface of a pathogen, and catalyzes the hydrolysis of C3 in the blood into C3a and C3b, which positively affects the number of C3bBb hooked onto a pathogen. After hydrolysis of C3, C3b complexes to become C3bBbC3b, which cleaves C5 into C5a and C5b. C5b with C6, C7, C8, and C9 (C5b6789) complex to form the membrane attack complex, also known as MAC, which is inserted into the cell membrane, which causes a “hole” to form in the membrane to initiate cells lysis. C5a and C3a are known to trigger mast cell degranulation. IgA is associated with activating the alternative path.

The lectin pathway is homologous to the classical pathway, but with the opsonin, mannose-binding lectin (MBL), and ficolins, instead of C1q. This pathway may be activated by binding mannose-binding lectin to mannose residues on the pathogen surface, which activates the MBL-associated serine proteases, MASP-1, and MASP-2 (very similar to C1r and C1s, respectively), which can then split C4 into C4a and C4b and C2 into C2a and C2b. C4b and C2a then bind together to form the C3-convertase, as in the classical pathway. Ficolins are homologous to MBL and function via MASP in a similar way. In invertebrates without an adaptive immune system, ficolins are expanded and their binding specificities diversified to compensate for the lack of pathogen-specific recognition molecules.

The complement system has the potential to be extremely damaging to host tissues, meaning its activation must be tightly regulated. The complement system is regulated by complement control proteins, which are present at a higher concentration in the blood plasma than the complement proteins themselves. Some complement control proteins are present on the membranes of self-cells preventing them from being targeted by complement. One example is CD59, also known as protectin which inhibits C9 polymerization during the formation of the membrane attack complex.

In the classical pathway, C1 may bind with its C1q subunits to Fc fragments (made of CH2 region) of IgG or IgM which has formed a complex with antigens. C4b and C3b may also bind to antigen-associated IgG or IgM, to its Fc portion. Such immunoglobulin-mediated binding of the complement may be interpreted as that the complement uses the ability of the immunoglobulin to detect and bind to non-self antigens as its guiding stick. The complement itself may bind non-self pathogens after detecting their pathogen-associated molecular patterns (PAMPs), however, utilizing specificity of antibody, complements are able to detect non-self enemies much more specifically. Some components have a variety of binding sites. In the classical pathway C4 may bind to Ig-associated C1q and C1r2s2 enzyme cleave C4 to C4b and 4a. C4b binds to C1q, antigen-associated Ig (specifically to its Fc portion), and even to the microbe surface. C3b may bind to antigen-associated Ig and to the microbe surface. The ability of C3b to bind to antigen-associated Ig could work effectively against antigen-antibody immune complexes to make them soluble. This induction of complement cascade in response to anti-cancer antibodies may be employed to provide localized amplification of secondary payload targeting.

In yet another set of embodiments, the biological cascade may be the inflammatory cascade. Acute inflammation may be characterized by marked vascular changes, including vasodilation, increased permeability, and the slowing of blood flow, which are induced by the actions of various inflammatory mediators. Vasodilation typically occurs first at the arteriole level, progressing to the capillary level, and brings about a net increase in the amount of blood present, causing the redness and heat of inflammation. Increased permeability of the vessels may result in the movement of plasma into the tissues, with resultant stasis due to the increase in the concentration of the cells within blood, which may be characterized by enlarged vessels packed with cells. Stasis allows leukocytes to marginate (move) along the endothelium, which facilitates their recruitment into the tissues. Normal flowing blood prevents this, as the shearing force along the periphery of the vessels moves cells in the blood into the middle of the vessel.

In one set of embodiments, the receiving composition comprises an agent able to bind a product of a biological cascade. The agent may bind specifically and/or non-specifically. In some embodiments, the agent may be an antibody, nucleic acid, protein, peptide, carbohydrate, small molecule, fatty acid, lipid, etc. The agent may also be, in some embodiments, a targeting entity. A targeting entity is any entity that binds to a component associated with an organ, tissue, cell, subcellular locale, extracellular matrix component, or the like. For example, in certain embodiments, the receiving composition may comprise an agent able to recognize or bind to a selected site, for example, within a subject. As a specific non-limiting example, the receiving composition may include an antibody-conjugated chemotherapeutic drug, which can recognize a receptor at a selected site within a subject, e.g., within a tumor.

For example, the agent may be able to bind a final product of the coagulation cascade. As a specific example, one end product of the coagulation cascade is fibrin, which may be polymerized to form a blood clot. Accordingly, in some embodiments, the agent is able to bind to fibrin. For instance, the agent may be a fibrin-binding peptide, such as a peptide comprising or consisting essentially of the sequence G-Y-e-C-hyP-cY-G-L-C-Y-I-Q (SEQ ID NO: 1), where lower case letters refer to D-amino acids (i.e., “e” refer to D-amino acids (rather than the naturally-occurring L-amino acids), hyP stands for hydroxyproline, and cY stands for chlorotyrosine. Other examples include the fibrin-binding peptides (GLPCDYYGTCLD (SEQ ID NO: 13), GYLCGDYTLCPD (SEQ ID NO: 14), LPCDYYGTC-Bip (SEQ ID NO: 15), LPCDYYGTC-Bip-d (SEQ ID NO: 16), where Bip is L-biphenylalanine and d is the D-isomer of aspartic acid), Factor XIII-targeting ligands, fibronectin-targeting ligands (e.g., CGLIIQKNEC (SEQ ID NO: 17) or CNAGESSKNC (SEQ ID NO: 18)), fibrin and coagulation factor mimetics, or other ligands that accumulate in regions of coagulation. Still other examples include vascular disrupting agents (tubulin-binding drugs, cytokines, coagulation factors, cytokine-inducing drugs), platelet depletion, targeted coagulation activators from insect or reptile venom, inflammation-inducing drugs, heating, or other compounds that can induce coagulation in tumors.

As another example, the agent may be able to bind a final product of the activation cascade. Examples include, but are not limited to ligands (e.g., antibody, protein, peptide, small molecule, saccharide, or other chemical/biological moiety) able to bind to amplified receptors expressed during inflammation, including ICAM receptors, selectins (including P-selectin, E-selectin, and L-selectin), exposed parenchymal epitopes as a result of amplified vascular permeability (including generic receptors such as collagen and other matrix epitopes, or tumor and/or tissue-specific receptors), or any population of immune cells ushered into areas of inflammation.

The agent may also be able to bind a product of a complement cascade, as another example. These include any epitopes exposed as a result of localized complement activity targeted via peptides, antibodies, proteins, polysaccharides, or other vehicles. As another example, the agent may bind to an intermediate product of a biological cascade, for example, a protein or an enzyme within the biological cascade. For instance, the agent may bind to an enzyme, such as an enzyme which catalyzes a further reaction within the biological cascade. The agent may be, for example, a substrate that is recognizable by the enzyme, or an antibody or a peptide able to specifically bind the enzyme. As a specific example, Factor XIII is an enzyme within the coagulation cascade, which catalyzes fibrin polymerization. An agent such as a peptide recognizable by Factor XIII may be used as the agent, for instance, a peptide comprising the sequence GNQEQVSPLTLLKXC (SEQ ID NO: 2), where X is 6-aminohexanoic acid. In some cases, upon binding of the agent to the enzyme, a portion of the receiving composition may be cleaved, e.g., by the enzyme.

In one set of embodiments, the receiving composition includes a targeting entity. The targeting entity may be, for instance, a nucleic acid, a polypeptide, a glycoprotein, a carbohydrate, a lipid, etc. In some embodiments, a targeting entity may be a naturally occurring or synthetic ligand for a cell surface receptor, e.g., a growth factor, hormone, LDL, transferrin, etc. A targeting entity can be, for example, a nucleic acid targeting entity that binds to a cell type specific marker. The targeting entity may also be an antibody in some embodiments. In some embodiments, the targeting entity may be a peptide. The peptide can be identified, e.g., using known procedures such as phage display. As non-limiting examples, in some embodiments, the targeting entity may be a coagulation-specific targeting ligand, such as a fibrin-binding peptide or a peptide substrate for the transglutaminase Factor XIII.

In some embodiments, targeting entities are able to bind to an organ, a tissue, a cell, an extracellular matrix component, and/or an intracellular compartment that is associated with a specific developmental stage or a specific disease state (i.e. a “target” or “marker”). In some embodiments, a target is an antigen on the surface of a cell, such as a cell surface receptor, an integrin, a transmembrane protein, an ion channel, a membrane transport protein, etc. The target may also be an intracellular protein in some cases. In some embodiments, the target is a soluble protein, such as immunoglobulin. The target may be selected in some cases to be more prevalent, accessible, and/or abundant in a diseased locale or other selected site (e.g., organ, tissue, cell, subcellular locale, and/or extracellular matrix component), relative to a healthy locale. As a non-limiting example, in some embodiments, a target may be expressed to a greater extent in tumor tissues relative to normal tissues. In some embodiments, the target is more prevalent, accessible, and/or abundant in a selected site that is associated with a particular developmental state, relative to other locales having different developmental states. In some embodiments, targeting entities facilitate the passive entry into target sites by extending circulation time of conjugates, reducing non-specific clearance of conjugates, and/or geometrically enhancing the accumulation of conjugates in target sites.

In some embodiments, the targeting entity is a nucleic acid. As used herein, a “nucleic acid” targeting entity refers to a nucleic acid that binds selectively to a target. In some embodiments, the nucleic acid targeting entity is a nucleic acid aptamer. In general, an aptamer is an oligonucleotide (e.g., DNA, RNA, or an analog or derivative thereof) that binds to a particular target or target structure. An aptamer is typically a polynucleotide that binds to a specific target that is associated with a selected site. The targeting function of the aptamer can be based on the three-dimensional structure of the aptamer and/or target in some cases.

In some embodiments, the targeting entity in a protein or peptide. The protein or peptide may be of any suitable size or dimension. For instance, in certain embodiments, the peptide may have a size ranging from about 5 to 100, 10 to 75, 15 to 50, 20 to 25 amino acids, etc. The peptide sequence may have any useful lamino acid sequence, or in some cases, the peptide sequence may include a random arrangement of amino acids. Example proteins that may be used as targeting entities include, but are not limited to, antibodies, receptors, cytokines, peptide hormones, proteins derived from combinatorial libraries (e.g., avimers, affibodies, etc.), and/or characteristic portions thereof.

In some embodiments, a targeting entity may be a small molecule. Any small molecule that specifically binds a desired target can be used. In some embodiments, the targeting entity may be a carbohydrate (e.g., glycoproteins, proteoglycans, etc.). In some embodiments, the targeting entity may include one or more fatty acid groups or salts thereof (e.g., lipoproteins). In some embodiments, the targeting entity is targeted to a particular location by a property that is intrinsic to a particle (e.g., the geometry of the particle, assembly of multiple particle entities, etc.). In some embodiments, an agent of a receiving composition may function as a targeting entity as described herein. As a non-limiting example, an antibody that is useful for targeting tissues may also serve as a pharmaceutical agent.

As previously discussed, according to certain embodiments, the targeting entity is associated with a specific developmental stage or a specific disease state, i.e., a marker. Numerous markers are known in the art. Typical markers include cell surface proteins, e.g., receptors. Examples of receptors include, but are not limited to, the transferring receptor, LDL receptor, growth factor receptors such as epidermal growth factor receptor family members (e.g., EGFR, HER-2, HER-3, HER-4, HER-2/neu, etc.) or vascular endothelial growth factor receptors, cytokine receptors, cell adhesion molecules, integrins, selectins, CD molecules, etc. The marker can be, in some cases, a molecule that is present exclusively or in higher amounts on a malignant cell, e.g., a tumor cell, relative to normal tissue. For example, prostate-specific membrane antigen (PSMA) is often expressed at the surface of prostate cancer cells. Other examples of markers include endothelial cell markers or tumor markers. As another example, the marker may be a polypeptide that is expressed at higher levels on dividing than on non-dividing cells. Nucleolin is an example. For instance, the peptide known as F3 is a suitable targeting entity for targeting nucleolin.

In certain embodiments, a marker may be expressed in significant amounts mainly on one or a few cell types or in one or a few diseases. A cell type specific marker for a particular cell type can be expressed at levels at least 3 fold greater in that cell type than in a reference population of cells, for example, taken from a mixture containing cells from a plurality (e.g., 5 to 10 or more) of different tissues or organs in approximately equal amounts. In some embodiments, the cell type specific marker is present at levels at least 4 to 5 fold, between 5 to 10 fold, or more than 10-fold greater than its average expression in a reference population. Detection or measurement of a cell type specific marker may make it possible to distinguish the cell type or types of interest from cells of many, most, or all other types of cells.

In one set of embodiments, the receiving composition includes a diagnostic agent. For example, the diagnostic agent may be one that is determinable when administered to a subject, e.g., qualitatively and/or quantitatively. The diagnostic agent may be determined directly or indirectly within the subject. For instance, the diagnostic agent may be fluorescent, radioactive, electron-dense, or the like. The location of the diagnostic agent within the subject (for example, within a selected site) may thus be determined using any suitable technique. In some cases, a portion, or all, of a subject may be determined to determine the diagnostic agent within the subject.

In some embodiments, fluorescent and/or luminescent moieties can be used as a diagnostic agent, which include a variety of different organic or inorganic small molecules commonly referred to as “dyes,” “labels,” or “indicators.” Examples include, but are not limited to, fluorescein, rhodamine, acridine dyes, Alexa dyes, and cyanine dyes. Fluorescent and luminescent moieties may include a variety of naturally occurring proteins and derivatives thereof, such as genetically engineered variants. For example, fluorescent proteins may include green fluorescent protein (GFP), enhanced GFP, red, blue, yellow, cyan, and sapphire fluorescent proteins, reef coral fluorescent protein, etc. Luminescent proteins include luciferase, aequorin, or derivatives thereof. Fluorescence or luminescence can be detected using any suitable approach including, but not limited to, spectrometry, fluorescence microscopy, flow cytometry, etc.

Other non-limiting examples of diagnostic agents include gases (for example, for ultrasound imaging); commercially available imaging agents used in positron emissions tomography (PET), computer assisted tomography (CAT), single photon emission computerized tomography, X-ray, fluoroscopy, and magnetic resonance imaging (MRI); ultrasound contrast compounds, etc. Non-limiting examples of materials for use as contrast agents in MRI include gadolinium, iron, magnesium, manganese, copper, and chromium species. Examples of materials for CAT and X-ray imaging include, but are not limited to, heavy metal species such as iodine-based materials or barium-based materials. Examples of radioactive species used in radioactive compounds include, but are not limited to, ¹⁸F, ¹⁵O, ¹¹C, ^{99m}Tc, ⁵⁷Co, ⁶⁰Co, ¹²³I, ¹²⁵I, ¹³¹I, ¹¹¹In, or 32P. In some cases, the subject may be imaged to determine the location of the diagnostic agent within the subject, e.g., using techniques such as ultrasound, PET, CAT, single photon emission computerized tomography, X-ray, fluoroscopy, magnetic resonance imaging, etc.

In another set of embodiments, the receiving composition may include a pharmaceutical agent or a therapeutic agent. The pharmaceutical or therapeutic agent, when administered to a subject, can have a therapeutic effect and/or elicits a desired biological and/or pharmacological effect. The agent may be one that, when introduced to a subject, can be delivered systemically and/or locally, depending on the application. For instance, the composition may be targeted to a tissue, or proximate a tissue, for local delivery of the pharmaceutical agent. Example pharmaceutical agents to be delivered include, but are not limited to, small molecules, organic compounds, organometallic compounds, nucleic acids, proteins (including multimeric proteins, protein complexes, etc.), peptides, lipids, carbohydrates, hormones, metals, radioactive elements and compounds, drugs, vaccines, immunological agents, and/or combinations thereof.

In some embodiments, the pharmaceutical agent may include a nucleic acid, including DNA or RNA. For instance, the nucleic acids may be delivered to a subject for gene therapy purposes. As specific examples, the pharmaceutical agent may include functional RNAs (e.g. siRNAs, RNAis, and shRNAs, tRNAs, ribozymes, RNAs used for triple helix formation, etc.). In some embodiments, the functional RNA is a ribozyme. In certain embodiments, one or more particles are used to deliver functional RNAs to a selected site, such as a tissue, cell, or subcellular locale. Example RNAs are described in U.S. Published Patent Application 2008/0213377, published Sep. 4, 2008, entitled “Delivery of Nanoparticles and/or Agents to Cells,” by Bhatia, et al., hereby incorporated by reference in its entirety.

The pharmaceutical agent may also be a small molecule and/or organic compound with pharmaceutical activity (such as a clinically-used drug) in other embodiments. Examples of drugs include, but are not limited to, antibiotics, anti-viral agents, anesthetics, antitumor species, inhibitors of an enzyme, steroidal agents, anti-neoplastic agents, anti-angiogenic agents, antigens, vaccines, antibodies, decongestants, antihypertensives, sedatives, progestational agents, anti-cholinergics, analgesics, beta-adrenergic blocking agents, diuretics, cardiovascular active agents, vasoactive agents, etc. The pharmaceutical agent, in other embodiments, can include a protein or peptide. The peptides may range from about 5 to about 40, about 10 to about 35, about 15 to about 30, about 20 to about 25 amino acids, etc. in size. In yet other embodiments, the pharmaceutical agent may include an antibody. In some embodiments, the pharmaceutical agent includes one or more prophylactic agents, such as vaccines. Vaccines may include isolated proteins or peptides, inactivated organisms and viruses, dead organisms and virus, genetically altered organisms or viruses, and cell extracts. Prophylactic agents may be combined with interleukins, interferon, cytokines, and adjuvants such as cholera toxin, alum, Freund's adjuvant, etc. These antigens may be in the form of whole killed organisms, peptides, proteins, glycoproteins, carbohydrates, etc.

In some embodiments, the receiving composition includes a mixture of pharmaceutical agents, including mixtures including any of the pharmaceutical agents described herein. As a non-limiting example, anesthetics may also be administered with vasoactive agents such as epinephrine. As another example, an antibiotic may be combined with an inhibitor of the enzyme commonly produced by bacteria to inactivate the antibiotic (e.g., penicillin and clavulanic acid, etc.).

In one set of embodiments, the receiving composition includes a liposome or a micelle. The term “liposomes” refers to artificial microscopic spherical particles formed by a lipid-containing bilayer (or multilayers) enclosing an aqueous compartment. The liposome may be unilamellar or multilamellar. Liposomes are usually nontoxic, physiologically acceptable, and are relatively simple to make and administer. Liposomes can be filled with drugs, and used to deliver drugs for cancer and other diseases. Liposomes may be formed from phospholipids or other lipids. Phospholipids are molecules that have a head group and a tail group. The head is attracted to water, and the tail, which is made of a long hydrocarbon chain, is repelled by water. Phospholipids are often found as stable membranes composed of two layers (a bilayer). In the presence of water, the heads are attracted to water and line up to form a surface facing the water. The tails are repelled by water, and line up to form a surface away from the water. The hydrocarbon tails of one layer face the hydrocarbon tails of the other layer, and the combined structure forms a bilayer. Examples of phospholipids include phosphatidylethanolamine and phosphatidylcholine. Other non-limiting examples include naturally-derived phospholipids with mixed lipid chains (like egg phosphatidylethanolamine), or of pure surfactant components like DOPE (dioleoylphosphatidylethanolamine).

In another set of embodiments, the receiving composition includes a particle. The particle may be, e.g., a microparticle or a nanoparticle, and can be spherical or non-spherical. The particles can be biodegradable and/or biocompatible in some cases. In some embodiments, the particle may have a greatest dimension (e.g., diameter) of less than about 1000 micrometers (μm). Further discussion of suitable particles can be seen below.

In certain embodiments, the particle may include one or more entities that offer protection, for example, against degradation or damage. For instance, the particles may have a coating layer. Use of a biocompatible coating layer can be advantageous, e.g., if the particles contain materials that are toxic to cells. The coating may be conjugated, linked, attached, tethered, etc. to the particle, and the coating may be bound covalently and/or noncovalently. The coating may be applied or assembled in a variety of ways such as by dipping, using a layer-by-layer technique, by self-assembly, conjugation, etc. Self-assembly refers to a process of spontaneous assembly of a higher order structure that relies on the natural attraction of the components of the higher order structure (e.g., molecules) for each other. It typically occurs through random movements of the molecules and formation of bonds based on size, shape, composition, and/or chemical properties. In some embodiments, the particles may include one or more optically or magnetically detectable materials forming a coating layer.

Examples of coating materials include, but are not limited to, natural proteins such as bovine serum albumin (BSA), biocompatible hydrophilic polymers such as poly(ethylene glycol) (PEG) or a PEG derivative, phospholipid (PEG), silica, lipids, polymers, and carbohydrates such as dextran. In some embodiments, PEG-coated particles may be useful, since such particles may exhibit reduced toxicity, enhanced protection from proteolytic degradation, or extended circulating life. In some embodiments, the particles may include a coating of poly(ethylene glycol) (PEG). In some embodiments, PEG is covalently associated with the particles; for example, the PEG may be covalently linked using a linker, such as a peptide linker. Examples of linkers are discussed herein.

The receiving composition may include a modulating entity, according to one set of embodiments. For example, the receiving composition may include one or more entities that modulate (e.g., control) their delivery and/or their activity, and/or protect them while in transit. The modulating entity may be associated with the receiving composition, or a portion thereof, e.g., with an agent able to bind a product of a biological cascade. As used herein, the term “modulating entity” refers to any entity that can be used to alter or affect delivery and/or efficacy of a moiety (such as a receiving composition, or a portion thereof, such as a diagnostic agent or a pharmaceutical agent), protect the moiety while in transit, and/or control the delivery and/or efficacy of the moiety. In certain embodiments, a modulating entity is a targeting moiety. In some embodiments, modulating entities are any entities that alter or affect the fate of the moiety within the body. For example, modulating entities may alter or affect the final tissue, cellular, or subcellular distribution of the moiety. Alternatively or additionally, modulating entities may direct a moiety to certain organs and/or tissues for excretion and/or breakdown. In some embodiments, modulating entities can protect the moiety, increase the stability of the moiety, increase the half-life of the moiety, increase the circulation time of the moiety, or the like. For example, the modulating entity may be poly(ethylene glycol), a transfection reagent (e.g., a dendrimer), a translocation entity, an entity that alters activity of a moiety to be delivered, an entity that mediates controlled release of a moiety, or an endosomal escape agent.

In another set of embodiments, a coagulation inhibitor may be applied to the subject. The coagulation inhibitor may be applied separately, and/or the coagulation may form part of the receiving composition. For example, the coagulation inhibitor may be an inhibitor able to inhibit fibrin formation or platelet self-assembly. In some cases, for example, where the coagulation cascade is activated, inhibition of coagulation may include effectiveness of the receiving composition, since coagulation is inhibited from occurring (which could reduce or eliminate blood flow) while the coagulation cascade is activated. As other examples, platelet self-assembly can be inhibited while allowing coagulation cascade use, or thrombin can be inhibited while allowing platelet self-assembly.

In one set of embodiments, the receiving composition may comprise one or more transfection reagents. As used herein, the term “transfection reagent” refers to any substance that enhances the transfer or uptake of an exogenous nucleic acid into a cell when the cell is contacted with the nucleic acid in the presence of the transfection reagent. In some embodiments, transfection reagents enhance the transfer of an exogenous nucleic acid, e.g. RNA, into mammalian cells. The transfection reagents may be employed to alter intracellular delivery of the receiving composition. In certain embodiments, the receiving composition may also comprise a functional RNA, such as an siRNA, which is deliverable to the subject. A variety of different transfection reagents can be used to alter delivery of large DNA molecules (typically several hundred to thousands of base pairs in length), which differ significantly in terms of structure from small RNA species such as short RNAi agents and tRNAs. Certain of these transfection reagents can mediate intracellular delivery of short RNAi agents and/or tRNAs. Examples of transfection reagents are disclosed in detail in U.S. Published Patent Application 2008/0213377, published Sep. 4, 2008, entitled “Delivery of Nanoparticles and/or Agents to Cells,” by Bhatia, et al., hereby incorporated by reference in its entirety.

In another set of embodiments, the receiving composition may comprise one or more translocation entities. Translocation entities may include peptides, proteins, glycoproteins, nucleic acids, carbohydrates, lipids, small molecules, etc. Typically, a translocation entity is a peptide. A translocation peptide can be any of a variety of protein domains that are capable of inducing or enhancing translocation of an associated moiety into a eukaryotic cell, e.g., a mammalian cell. For example, the presence of these domains within a larger protein may enhance transport of the larger protein into cells. These domains are sometimes referred to as protein transduction domains (PTDs) or cell penetrating peptides (CPPs). Translocation peptides include peptides derived from various viruses, DNA binding segments of leucine zipper proteins, synthetic arginine-rich peptides, etc. In some embodiments, translocation-enhancing moieties of use include peptide-like molecules known as peptoid molecular transporters. Certain of these molecules contain contiguous, highly basic subunits, particularly subunits containing guanidyl or amidinyl moieties.

In still another set of embodiments, the receiving composition may include one or more entities that alter the activity of an agent of the receiving composition. For instance, in some embodiments, such entities may include cationic reagents, for example, cationic polymers such as PEI, poly(lysine), or protamine, which may enhance activities of polynucleotides in cells.

In yet another set of embodiments, the receiving composition may include one or more entities that mediate controlled release of an agent. For example, in some cases, protease-cleavable peptides may be used. Cleavage can occur the sites where corresponding proteases are present. Proteases such as matrix metalloproteases (MMPs) are upregulated in many types of tumors. In some embodiments, agents to be delivered that are conjugated to particles via protease-cleavable bonds may be released from a receiving composition when it reaches tumor sites in vivo. Such agents (e.g., siRNAs, drugs, etc.) can be associated with the receiving composition using a protease-sensitive sequence. Serine proteases or MMPs have specific peptide sequences that they typically recognize and cleave. In some embodiments, one end of the target peptide is conjugated to a particle (covalently or non-covalently), with the other end conjugated to an agent (covalently or non-covalently). In some embodiments, heterobifunctional crosslinkers (e.g., sulfo-SPDP or sulfo-SMCC) are used to conjugate an amino group on one species (e.g. particle) to a thiol group on the other (e.g., cysteine residue on the peptide). In some cases, a target conjugate can be linked to a receiving composition with an additional conjugation step (e.g., a lysine residue on the peptide can be reacted with sulfo-SMCC to form a maleimide, which in turn can react with a thiol group added to the agent). Appropriate peptide sequences can be produced synthetically or expressed in a cell culture system. Purification can be performed to ensure that only the sequence of interest is conjugated between the nanoparticle and agent. Example peptide sequences and proteases that target these sequences are presented in Table 1:

| Target | | | |
|---|---|---|---|
| protease | Disease | Substrate Peptide | |
| Cathepsin B | Cancer | K•K | (SEQ ID NO: 4) |
| PSA | Prostate | HSSKLQ• | (SEQ ID NO: 5) |
| | cancer | | |
| Cathepsin D | Breast | PICF•F | (SEQ ID NO: 6) |
| | cancer | | |
| MMP-2 | Metastases | GPLG•VRG | (SEQ ID NO: 7) |
| HIV | HIV | GVSQNY•PIVG | (SEQ ID NO: 8) |
| protease | | | |
| HSV | HSV | LVLA•SSSFGY | (SEQ ID NO: 9) |
| protease | | | |
| Caspase-3 | Apoptosis | DEVD• | (SEQ ID NO: 10) |
| Caspase-1 | Apoptosis | WEHD• | (SEQ ID NO: 11) |
| (ICE) | | | |

In some embodiments, other proteases that can serve as target proteases include, but are not limited to, any matrix metalloprotease (e.g., MMP-1, MMP-7, MMP-9, MMP-13, etc.), caspase-2, NF-kappa B, Cathespin S, Cathespin K, etc. For instance, when a receiving composition is introduced into a region of high protease expression (e.g., targeted to tumor interstitium where a high concentration of MMPs are present), extracellular cleavage can cause separation to occur.

As mentioned, various aspects of the invention are directed to enhancing and/or localizing delivery of certain compositions or components to a selected site within a subject. The selected site can include any site at which treatment and/or diagnosis (e.g., imaging) is desired to occur. In some embodiments, the selected site is an area (e.g., a tumor) or a surrounding or proximate area that is affected by a disease (e.g., cancer). In some embodiments, the selected site includes blood vessels (e.g., tumor vasculature). For example, in cancer therapy, the vasculature of solid tumors can be the selected site for targeted therapy. Local interruption of the tumor vasculature can produce an avalanche of cell death because tumor cells depend on a blood supply. Other examples of selected sites include, but are not limited to, the heart, the liver, a site of infection, a fibrous site, etc.

As a non-limiting example, in some embodiments, coagulation or clotting can be induced to provide a change at a selected site. A coagulation cascade can be activated through intrinsic and/or extrinsic pathway activation, and/or through other techniques. For instance, as discussed herein, a local change in temperature (e.g., heating), particulate material, and non-particulate material can induce coagulation. For example, tumor-targeted plasmonic gold nanorods can activate coagulation by photothermally disrupting tumor vessels and/or exposing extravascular tissue factor or collagen to activate extrinsic and/or intrinsic coagulation pathways. As more examples, a coagulation cascade can be activated by tissue factor protein, or a molecule such as a photosensitizer that is capable of inducing thrombosis of blood vessels.

In some embodiments, a change at a selected site can include tissue inflammation and/or inflammation in a blood vessel. Inflammation is a localized protective response elicited by injury or destruction of tissues, which serves to destroy, dilute, or wall off both the injurious agent or event, and the injured tissue. Furthermore, both inflammation and coagulation can play important roles in the pathogenesis of vascular disease. In some cases, there may be cross-talk between inflammation and coagulation, in which inflammation may lead to activation of coagulation, and/or coagulation may lead to activation of inflammation.

Changes at a selected site can involve, in some embodiments, an increase in a local temperature, for example, when heat is locally generated. For example, local tumor heating can dilate tumor blood vessels and transvascular pores. Heat-induced vessel dilation can increase the exposure of blood proteins to the tumor parenchyma, stimulating a clotting cascade and rapid polymerization of fibrin in the intra- and extra-vascular spaces. This induced coagulation can be targeted in some cases.

In some embodiments, the permeability at or around a selected site can be changed. As discussed herein, heating can induce a permeability change of or near a selected site (e.g., tumor blood vessels). For example, cytokines (e.g., IL-2) can enhance vasopermeability. For IL-2, it is believed that the induced vasopermeability enhancement is caused by the release of nitric oxide gas within the milieu of the tumor or inflammatory lesion resulting in the rounding up of the endothelial cell wall and the generation of microfenestrations that enable leakage of fluid into affected tissues.

Another aspect of the invention generally involves activating the biological cascade, e.g., with an activation composition and/or by applying energy or another external stimulus, for example, heat, electromagnetic radiation, magnetism, or the like. Activation of the biological cascade may be prior to, simultaneously with, and/or after administering a receiving composition, for example, comprising an agent able to bind a product of a biological cascade as discussed herein. Activation of the biological cascade may initiate or facilitate association of the receiving composition with the biological cascade. For example, activation of a biological cascade may result in coagulating a selected site, inflaming the selected site, changing the permeability of the selected site, changing the porosity of the selected site, changing a local concentration of a compound capable of being targeted by the activation component, etc. The activation composition may be a biomolecule or a small molecule that can change a selected site from first state to second state chemically, biologically, and/or physically. In some cases, the activation composition may have dimensions sufficiently small to allow uptake by eukaryotic cells. For example, the activation composition may include particles such as nanoparticles, or other compositions as described below.

In one set of embodiments, the biological cascade may be activated by applying energy, e.g., to a selected site. Optionally, an activation composition may also be delivered to the subject, which in some cases may interact with the applied energy to activate the biological cascade. For example, a selected site can be induced to coagulate by locally heating the selected site using stimuli such as electromagnetic radiation, infrared heating, high intensity focused ultrasound, radiofrequency ablation, radiofrequency hyperthermia, laser ablation, and/or external application of heat. Devices and applicators able to apply such stimuli can be readily obtained commercially. As another example, vessel damage to a selected site can be caused by using stimuli such as ultrasound-induced cavitation of microbubbles. An inflammatory response can also be induced, in some embodiments, by a stimulus such as X-rays, radioactive radiation, and/or an electromagnetic field. As yet other examples, a biological cascade may be activated by applying electromagnetic radiation, e.g., visible light, infrared radiation, ultraviolet radiation, X-rays, etc. In yet another example, a biological cascade such as the coagulation cascade may be activated using externally applied force or pressure, by physically cutting or abrading the selected site (e.g., with a probe, a scalpel, a blade, etc.), etc.

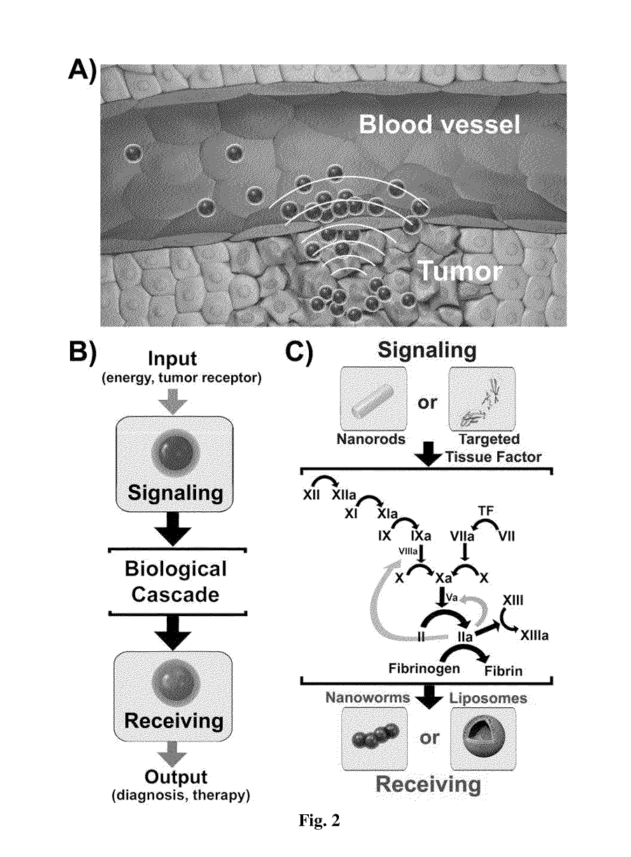

The biological cascade may be activated using an activation composition in another set of embodiments. The activation composition may comprise a reactant within a biological cascade (e.g., one whose presence causes or initiates reactions within the biological cascade), an enzyme or other catalyst within the biological cascade, or in some cases, the activation composition may be a trigger that is recognized by the biological cascade, thereby activating the biological cascade. As a specific non-limiting example, referring to the coagulation cascade shown in FIG. 2B, the coagulation cascade may be activated by providing a reactant of the biological cascade (e.g., fibrin), providing a catalyst within the biological cascade (e.g., activated Factor XIII), or by damaging tissue in the subject (e.g., which is able to trigger coagulation within the subject through a variety of mechanisms, including the release of tissue factor protein.

Referring again to the example shown in FIG. 1B, in this example, first component 104 can include any material capable of changing selected site 108 from a first state to a second state different from the first state. For example, first component 104 may be capable of changing a chemical, a biological, and/or a physical property of selected site 108. As a specific example, for a chemical change, first component 104 may cause a local increase in concentration of a chemical species that can bind (e.g., selectively or preferentially) with second component 106. For instance, certain receptors and proteases such as MMPs can be upregulated in many types of tumors. As a non-limiting example of a biological change, first component 104 may produce coagulation or clotting in at least a portion of selected site 108. As an example of a physical change, first component 104 may change (e.g., increase) porosity of or near selected site 108. In some embodiments, first component 104 can change selected site 108 by itself and/or by interacting with other materials. In some cases, changes caused by an activation composition may be caused by the activation composition receiving information in the form of, for example, a biological signal and/or an exogenous stimulus, or other applied energy (such as an electromagnetic stimulus). The second condition may be selected in some cases to be targeted by the activation composition to enhance delivery of a receiving composition to the selected site.

In some embodiments, an activation composition may be characterized by an effect of the activation composition can cause on a selected site within a subject. The effect can be, for instance, a stressful change or a benign change to a tissue. A stressful change can include the initiation of any process that induces responses that lower the apoptotic threshold of target cells or tissues or re-arranges the target tissue structure for the purpose of increasing the tissues visibility to other system components. As examples of changes to a selected site, the activation composition can cause coagulation or clotting, inflammation, a change in permeability, a change in porosity, a change in the local concentration of a chemical species, vessel damage, complement cascade activation, activation of other biological cascades, modification of endothelial or parenchymal cell surface, heat-shock protein responses, internal stress responses, pre-apoptotic or apoptotic changes in cells, matrix reorganization or cleavage of matrix components, any gene expression or protein localization changes in the target tissue, recruitment of immune or other cells, or any combination of such effects.

In some embodiments, the changes can be produced by delivering an exogenous stimulus or stimuli to a selected site, for example, an activation composition. The activation composition can be particulate or non-particulate. For example, the activation composition can be in the form of one or more particles, or in the form of non-particles, such as small molecules (e.g., a therapeutic agent or a drug) or biomolecules (e.g., a protein or a peptide), etc. In some embodiments, a particulate and a non-particulate activation composition are used together.

In certain embodiments, the activation composition includes (e.g., is formed wholly of) a material, such as an inorganic material, that can be activated as a heat source. For example, certain inorganic materials, such as plasmon resonant materials (e.g., gold, silver, copper, and materials including these metals) or certain semiconductors, are capable of absorbing energy (e.g., infrared radiation) to produce heat. A specific example is gold nanorods, which can release heat when exposed to certain types of light. Other materials are capable of absorbing radiofrequency (RF) energy to produce heat. Still other materials (such as superparamagnetic iron oxide) are capable of interacting with a magnetic or an electromagnetic field, such as a magnetic resonance frequency field, to produce heat (e.g., by induction according to Faraday's Law). By exposing these materials to the appropriate stimulus (e.g., optical energy, RF energy, electromagnetic field, etc.), materials such as these can provide targeted and local heating (e.g., an increase of tens of degrees) without necessarily causing systemic heating. For instance, such local heating can cause a selected site to coagulate selectively, and this coagulation can then be targeted by a receiving composition, as described herein.

As a non-limiting example of a class of materials that can produce heat by exposure to an appropriate stimulus, an activation composition can include one or more magnetic particles (e.g., nanoparticles or microparticles). In some embodiments, magnetic particles experience heating due to Brownian relaxation and reorientation of their magnetic poles. The magnetic particles may include one or more ferrimagnetic, ferromagnetic, paramagnetic, and/or superparamagnetic materials. The magnetic particles may include (wholly or in part) of one or more of the following materials: iron, cobalt, nickel, niobium, magnetic iron oxides (such as maghemite (γ-Fe₂O₃) or magnetite (Fe₃O₄)); hydroxides (such as feroxyhyte (FeO(OH))); double oxides or hydroxides of two- or three-valent iron with two- or three-valent other metal ions, such as those from the first row of transition metals (e.g., Co(II), Mn(II), Cu(II), Ni(II), Cr(III)); and mixtures of any of the foregoing. Additional materials that may be included in magnetic particles include, but are not limited to, yttrium, europium, gadolinium, dysprosium, samarium, and vanadium.

In some embodiments, the activation composition may include inorganic materials and/or organic materials. Non-limiting examples of inorganic materials include metals (e.g., gold, silver, copper, platinum, palladium, etc.), alloys, oxides, and hydroxides. Examples of organic materials include polymers. In some embodiments, the activation composition may include a composite containing an inorganic material and an organic material. For example, composite particles can include a plurality of metal particles that are embedded in or encapsulated by a polymer, such as iron oxide nanoparticles or gold nanorods encapsulated by a biodegradable polymer (e.g., poly(amino esters) or a non-biodegradable (e.g., polyacrylamide) polymer.

In some embodiments, the activation composition may include one or more synthetic particles and a delivery vehicle comprising biocompatible materials, such as micelles, liposomes, dendrimers and polymers including organic, inorganic (e.g., silica) and/or hybrid (e.g., organic modified silica) polymers. In some embodiments, the delivery vehicle is a polymeric nanoparticle. In other embodiments, the delivery vehicle is a linear polymer. These synthetic particles and delivery vehicles are discussed further elsewhere herein.

In some embodiments, the particles are capable of exhibiting self-assembly. The assembly of conjugates and/or particles can induce a change in biological, chemical, and/or physical properties on a selected site. For example, self-assembly of particles accumulated at tumor site can result in activation of coagulation and/or inflammation in tumor blood vessels.

In one set of embodiments, the activation composition may be able to cause a selected site to change from a first condition to a second condition. If particles are used, biomolecules and/or small molecules that are used as part of the activation composition can be embedded in the particles (e.g., for degradable polymeric particles) and/or placed on the surface of the particles (e.g., for certain inorganic particles).

For example, to cause coagulation, an activation composition can include one or more biomolecules (such as a tissue factor protein, TNF-alpha, or other cytokines, or endothelially-targeted antibodies) and/or one or more small molecules (such as endothelial cell destabilizing agents (including combrestatin-like molecules) or any vascular disrupting agents) capable of causing coagulation at the selected site.

Non-limiting examples of activation compositions able to induce coagulation include NGR-IFNgamma, NPI2358 with docetaxel (docetaxel and plinabulin), NPI2358 with Folfiri (fluorouracil, irinotecan, leucovorin, and plinabulin), Zybrestat (fosbretabulin disodium), STA9584, VEGF/rGel-Op, BNC105, Arenegyr, Arenegyr with cisplatin, Arenegyr with doxorubicin, NPI2358 (plinabulin), Zybrestat with Avastin (bevacizumab and fosbretabulin disodium), Zybrestat with Cetuximab (cetuximab and fosbretabulin disodium), ASA404 with docetaxel (dimethylxanthenone acetic acid and docetaxel), CYT997, CYT997 with carboplatin and etoposide, Azixa, Azixa with carboplatin, Azixa with temozolomide, Arenegyr with Xelox (capecitabine and oxaliplatin), OXi4503 (combretastatin A-1 diphosphate), Zybrestat with (131) I-A5B7 (carcinoembryonic antigen monoclonal antibody and fosbretabulin disodium), Zybrestat with paclitaxel and carboplatin (carboplatin, fosbretabulin disodium, and paclitaxel), AVE8062, ASA404 with Erbitux (dimethylxanthenone acetic acid and cetuximab), AZD4440, ZD6126 (N-acetylcolchinol phosphate), AS1404 (dimethylxanthenone acetic acid), EndoTAG-2 (camptothecin), OXi8007, Zybrestat with cisplatin (cisplatin and fosbretabulin disodium), VEGF121/rGel, TVT-Dox (doxorubicin), NGR-IL12, and ASA404 (dimethylxanthenone acetic acid), as well as combinations of these and/or other compounds.

Other non-limiting examples of activation compositions include bradykinin from the kinin system, which is a vasoactive protein which is able to induce vasodilation, increase vascular permeability, cause smooth muscle contraction, and induce pain; C3 from the complement cascade, which cleaves to produce C3a and C3b; C5a from the complement cascade, which stimulates histamine release by mast cells, thereby producing vasodilation, and/or acting as a chemoattractant to direct cells via chemotaxis to the site of inflammation; Factor XII (Hageman Factor), which is a protein that circulates inactively, until activated by collagen, platelets, or exposed basement membranes via conformational change, and when activated, it can activate plasma systems involved in inflammation, including the kinin system, fibrinolysis system, and coagulation system; or plasmin, which is able to break down fibrin clots, cleave complement protein C3, and activate Factor XII.

Similarly, to inflame a selected site, an activation composition may include one or more biomolecules (TNF-alpha, interferons, interleukins, or other cytokines, endo- and exo-toxins (including lipopolysaccharides)), polynucleotide-based inflammatory mediators (including DNA and RNA motifs), pro-inflammatory hormones (prostaglandins, histamines), bacteria or viruses and components thereof, endothelially-targeted or any tissue-targeted antibodies) and/or one or more small molecules (such as prostaglandins, histamines, leukotrienes, and any other chemicals capable of inducing hallmark features of inflammation, including enhanced vessel permeability, leukocyte recruitment, and other features) capable of inflaming the selected site.

Examples of suitable cell-derived mediators which can be used as activation compositions include, but are not limited to, lysosome granules (granulocytes); histamine (mast cells, basophils, platelets); IFN-gamma; IL-8, leukotriene B4; nitric oxide; prostaglandins, TNF-alpha, or IL-11. Lysosome granules are produced in granulocytes, which contain a large variety of enzymes which perform a number of functions. Granules can be classified as either specific or azurophilic depending upon the contents, and are able to break down a number of substances, some of which may be plasma-derived proteins which allow these enzymes to act as inflammatory mediators. Histamine is stored in preformed granules in mast cells, basophils, orplatelets. Histamine is released in response to a number of stimuli. It causes arteriole dilation and increased venous permeability. IFN-gamma has antiviral, immunoregulatory, or anti-tumor properties. This interferon was originally called macrophage-activating factor, and is important in the maintenance of chronic inflammation. IL-8 causes activation and chemoattraction of neutrophils, with a weak effect on monocytes and eosinophils. Leukotriene B4 is able to mediate leukocyte adhesion and activation, allowing them to bind to the endothelium and migrate across it. In neutrophils, it is also a chemoattractant, and is able to induce the formation of reactive oxygen species and the release of lysosome enzymes by these cells. Nitric oxide is a potent vasodilator. It may also relax smooth muscle, reduce platelet aggregation, aid in leukocyte recruitment, or direct antimicrobial activity in high concentrations. Prostaglandins are lipids that can cause vasodilation, fever, or pain. TNF-alpha and IL-1 can affect a wide variety of cells to induce many similar inflammatory reactions, including fever, production of cytokines, endothelial gene regulation, chemotaxis, leukocyte adherence, or activation of fibroblasts. These are also able to affect certain systemic effects of inflammation, such as loss of appetite and increased heart rate.

To change the permeability or porosity of a selected site, an activation composition may include one or more biomolecules (such as TNF-alpha, which can destroy tumor vasculature selectively and enhance the tumor vascular permeability) and/or one or more small molecules (such as prostaglandins, histamines, leukotrienes and others) capable of making the selected site more or less permeable or porous.

To improve delivery to a selected site, in one set of embodiments, an activation composition can include one or more modulating entities (e.g., poly(ethylene glycol) (PEG)). Examples of modulating entities include targeting entities, transfection reagents, translocation entities, endosome escape entities, entities that alter activity of materials, entities that mediate controlled release of materials, etc. As a non-limiting example, PEG-coated gold nanorods (PEG-NRs) have relatively long circulation times in mice, which may allow passive targeting to tumors via their fenestrated blood vessels and precise tumor heating using near-infrared (NIR) energy. In some embodiments, a modulating entity is a targeting entity that directs a particle to a specific tissue, cell, or subcellular locale.

In some embodiments, an activation composition can include functional agents, such as optically detectable (e.g., quantum dots) and/or magnetically detectable particles (e.g., iron-containing particles) or molecules (e.g. fluorescent dyes) or drugs. For example, an activation composition can have one or more particles capable of being heat sources, an imaging agent (e.g., a fluorescent dye or an MRI contrast agent) and/or a targeting entity for targeted delivery.

In one set of embodiments, the activation composition can be delivered in vivo, and in some cases can have dimensions sufficiently small to allow uptake by eukaryotic cells. Non-limiting examples include particles such as nanoparticles (e.g., particles having a mean dimension smaller than approximately 1,000 nm), microparticles (e.g., particles having a mean dimension larger than approximately 1 micron), dendrimers, micelles, liposomes, etc. The nanoparticles may also have a largest dimension that is smaller than approximately 200 nm, smaller than approximately 100 nm, or smaller than approximately 50 nm (e.g., approximately 5-30 nm). In some embodiments, the particles may be characterized by an Enhanced Permeability and Retention (EPR) effect. Such enhanced accumulation in tumors may be caused by the increased permeability of angiogenic tumor vasculature relative to normal vasculature. In some cases, the particles can diffuse through such “leaky” vasculature, resulting in accumulation of particles in tumors.

Various examples of activation compositions follow, and an activation composition can include one or more of these examples, and/or other agents. For example, the activation composition for activating a biological cascade may be capable of increasing the concentration of a receptor, enzyme, or biochemical/mechanical characteristic of a tumor, e.g., via indirect signaling to endogenous processes (of inflammation, stress responses, or other immune or biological responses) or via direct expression of said receptor, enzyme, or characteristic themselves) that are targeted. In certain embodiments, the activation composition includes TNF-alpha.

In one set of embodiments, the activation composition is tissue factor protein. Tissue factor protein is a membrane-bound protease on extravascular cell surfaces that can activate the extrinsic clotting cascade. When truncated to remove the membrane-bound region, its activity drops by about five orders of magnitude. This activity may be regained, for example, by directing its binding to the surface of a cell harboring phosphatidylserine lipids. By attaching a tumor-targeting domain to the tissue factor protein, its coagulation activity may be negligible in circulation and activated upon binding, e.g., to tumor vessels.

Other examples of potentially suitable activation compositions include microorganisms. Some examples of microorganisms may be infectious agents that can inflame a selected site, such as bacteria, viruses, fungi and other pathogenic or non-pathogenic microorganisms, naturally-occurring, genetically manipulated, or selected for via controlled evolution techniques, etc. Example bacteria include Escherichia coli, Pseudomonas aeruginosa, Staphylococcus aureus, and Mycobaterium tuberculosis. Example viruses include adenoviruses, herpes viruses, coxsackie viruses, HIV, rhinoviruses, comaviruses, and flu viruses. Example fungi include Candida albicans, Bacillus subtilis and Bacillus athrophaeus.

In another set of embodiments, the activation composition includes a human cell. In some cases, human cells (e.g., immune cells, stem cells, etc.) are isolated from the body, manipulated in some way, and re-injected; one or more of these could be programmed to target a selected site.

In yet another set of embodiments, the activation composition includes a small molecule. For instance, the small molecules may be molecules with low molecule weight that are capable of coagulating a selected site, inflaming the selected site, changing the permeability of the selected site, changing the porosity of the selected site, or changing a local concentration of a compound capable of being targeted by an activation composition. For example, a photosensitizer (e.g., Sn(IV) chlorin e6) that generates toxic oxygen species (e.g., singlet oxygen) after irradiation may be used to initiate coagulation in tumor blood vessels and further recruit the accumulation of an activation component for therapy or diagnosis. Other non-limiting examples of small molecules include, but are not limited to, prostaglandins, histamines, leukotrienes, which can inflame a selected site; cytokines (e.g., IL-2), prostaglandins, histamines, leukotrienes, which change the permeability and/or porosity of the selected site.

In some embodiments, the activation composition may include a targeting entity for delivery. Examples of targeting entities are discussed herein, and include those targeting entities discussed with reference to the receiving composition. For instance, in certain embodiments, the targeting entity may be able to recognize or bind to a selected site, for example, within a subject.

In some cases, the activation composition may be cloaked by a blocking agent that prevents the activation composition from interacting with a selected site. The activation composition may then be altered in some fashion to allow it to interact with the selected site, e.g., when the blocking agent is removed. Non-limiting examples of blocking agents include polaxamines, poloxamers, poly(ethylene glycol) (PEG), peptides, or other synthetic polymers of sufficient length and density (e.g., to mask self-assembly and provide protection against non-specific adsorption, opsonization, and reticulo-endothelial system (RES) uptake). In some embodiments, the blocking agent includes a PEG chain that is approximately 2.5 kDa, approximately 5 kDa, approximately 7.5 kDa, approximately 10 kDa, approximately 15 kDa, approximately 20 kDa, or approximately 25 kDa.

Various aspects of the invention involve particles, including activation compositions and/or receiving compositions. In some cases, the particles are microparticles or nanoparticles. In certain embodiments, the particle may be greater in size than the renal excretion limit (e.g., particles having diameters of greater than 6 nm), or small enough to avoid clearance of particles from the bloodstream by the liver (e.g., particles having diameters of less than 1000 nm). The physiochemical features of particles, including particle size, can be selected to allow a particle to circulate longer in plasma by decreasing renal excretion and/or liver clearance. Particles under 100 nm may be easily endocytosed. In some embodiments, particles under 400 nm may be characterized by enhanced accumulation in tumors. While not wishing to be bound by any theory, enhanced accumulation in tumors may be caused by the increased permeability of angiogenic tumor vasculature relative to normal vasculature. Particles can diffuse through such “leaky” vasculature, resulting in accumulation of particles in tumors and enhanced delivery of, for example, pharmaceutical agents and/or diagnostic agents.

In some embodiments, a particle may have a greatest dimension (e.g., diameter) of less than about 500 micrometers, less than about 300 micrometers, less than about 100 micrometers, less than about 50 micrometers, less than about 30 micrometers, less than about 10 micrometers, less than about 5 micrometers, less than about 3 micrometers, less than about 1000 nm, less than about 900 nm, about 800 nm, about 700 nm, about 600 nm, about 500 nm, about 400 nm, about 300 nm, about 200 nm, about 100 nm, about 50 nm, about 25 nm, or about 10 nm; and/or greater than or equal to about 1 nm, about 3 nm, about 10 nm, about 25 nm, about 50 nm, about 100 nm, about 200 nm, about 300 nm, about 400 nm, about 500 nm, about 600 nm, about 700 nm, about 800, about 900 nm, or about 1000 nm.

The particles, according to some embodiments of the invention, may have a variety of different shapes including, but not limited to, spheres, oblate spheroids, cylinders, ovals, ellipses, shells, cubes, cuboids, cones, pyramids, rods (e.g., cylinders or elongated structures having a square or rectangular cross-section), tetrapods (particles having four leg-like appendages), triangles, prisms, etc. The particles may be formed from any suitable material. For example, in certain embodiments, the particles include metal particles, silica particles, and/or polymeric polymers. Suitable metals can include, but are not limited to, gold, silver, iron, cobalt, zinc, cadmium, nickel, gadolinium, chromium, copper, manganese, palladium, tin, and alloys thereof. Oxides of any of these metals can be used. Certain lanthanide ion-doped nanoparticles exhibiting strong fluorescence can be used. A variety of different dopant molecules can be used in some cases. For example, in certain embodiments, the particles may include fluorescent europium-doped yttrium vanadate (YVO₄) nanoparticles. In some cases, the particles may be functionalized with biomolecules. In certain embodiments, the particles may include silica (SiO₂). The amount of silica in the particle, or in a core or coating layer including silica, can range from approximately 5% to 100% by mass, volume, or number of atoms, or can assume any value or range between 5% and 100%.

In some embodiments, the particles include an organic polymer. A wide variety of organic polymers and methods for forming nanoparticles therefrom may be used. For example, particles including poly(methylmethacrylate), poly(acrylamide), poly(vinyl chloride), carboxylated poly(vinyl chloride), or poly(vinyl chloride-co-vinyl acetate-co-vinyl alcohol) may be used. Optionally the particles include one or more plasticizers or additives. Co-polymers, block co-polymers, and/or grafted co-polymers can also be used in some embodiments. The polymers can be biodegradable or non-biodegradable. In some embodiments, the polymers are biocompatible.

The amount of inorganic material (e.g., metal(s) or magnetic material(s)) in a particle, in a core, and/or in a coating layer can range from approximately 5% to approximately 100% by mass, volume, or number of atoms. The amount of inorganic material can be greater than or equal to approximately 5%, approximately 10%, approximately 20%, approximately 30%, approximately 40%, approximately 50%, approximately 60%, approximately 70%, approximately 80%, or approximately 90% by mass, volume, or number of atoms; and/or less than or equal to approximately 100%, approximately 90%, approximately 80%, approximately 70%, approximately 60%, approximately 50%, approximately 40%, approximately 30%, approximately 20%, or approximately 10% by mass, volume, or number of atoms. The particles may include gradient or homogeneous alloys in certain embodiments. The particles may be composite particles in some cases including two or more materials, of which one, more than one, or all of the materials possess magnetic properties, electrically detectable properties, and/or optically detectable properties. In some embodiments, the particles include magnetic particles.

In some embodiments, a particle may have a composite structure. For example, a particle can be a composite particle having an inner core or layer containing a first material, and an outer layer or shell containing a second material. The particles may have a core/shell structure in some embodiments, wherein the core and shell can include different materials. The first and second material can include, in any combination, a metal (e.g., containing gold or silver), a metal-containing material (e.g., monocrystalline and/or superparamagnetic iron oxide), a nonmetallic material (e.g., a dielectric material such as silica), an organic material, etc. In some embodiments, at least one of the first and/or second materials is magnetic. In some embodiments, a magnetic particle contains a magnetic material and one or more nonmagnetic materials, which may be a metal or a nonmetal (e.g., quantum dots (QD), ceramics, polymers containing inorganic materials, bone-derived materials, bone substitutes, and viral particles). In certain embodiments, a particle may be coated with a metal shell that can absorb specific wavelengths of incident electromagnetic energy by varying particle diameter and shell thickness. The particles may be tuned to absorb specific frequencies of interest by altering the composition and/or the shape of the particle. For example, gold nanoparticles can absorb at approximately 520 nm when spherical, but rod-shapes or core-shell architectures can be tuned to absorb in the near infrared region of light (approximately 700 nm to approximately 1000 nm). The particles described herein can be solid throughout or hollow. Hollow particles (e.g., hollow nanoparticles coated with a shell) have an interior volume or cavity. In some embodiments, hollow particles having two or more concentric hollow spheres are used. A solid particle can include, for example, a core (e.g., made of a dielectric material) coated with a shell (e.g., a dielectric inorganic material or an organic material).

In some embodiments, a population of particles is relatively uniform in terms of size, shape, and/or composition so that each particle has similar properties. For example, at least about 80%, at least about 90%, or at least about 95% of the particles may have a diameter or greatest dimension that falls within about 5%, about 10%, or about 20% of the average diameter or greatest dimension. In some embodiments, however, a population of particles may be heterogeneous with respect to size, shape, and/or composition. In certain embodiments, one or more substantially uniform populations of particles is used, e.g., 2, 3, 4, 5, or more substantially uniform populations having distinguishable properties, e.g., optical and/or magnetic properties. For instance, the use of multiple distinguishable particle populations may allow tracking of multiple different agents or particles concurrently. A combination of two or more populations having distinguishable properties can be considered to be a single population of particles. In some embodiments, the particle may include one or more diagnostic agents and/or one or more pharmaceutical agents, as discussed herein.

In certain embodiments, a particle is porous. For instance, the particle may contain holes or channels that are relatively small compared with the size of a particle. For example, a particle may be a porous silica particle, e.g., a mesoporous silica nanoparticle or may have a coating of mesoporous silica. Particles may have pores ranging from about 1 nm to about 50 nm in diameter, e.g., between about 1 nm and 20 nm in diameter. Between about 10% and 95% of the volume of a particle may include voids within the pores or channels.

In some embodiments, the particles include one or more dispersion media, surfactants, release-retarding ingredients, or other pharmaceutically acceptable excipients, e.g., as described herein. In some embodiments, the particles include one or more plasticizers or additives.

In some embodiments, the particles may be magnetic. For example, the particles may include iron or other magnetic materials. Detection of magnetic particles administered to a subject may be performed using any suitable technique. For example, a magnetometer or a detector based on the phenomenon of magnetic resonance (NMR) can be employed. Superconducting quantum interference devices (SQUID), which use the properties of electron-pair wave coherence and Josephson junctions to detect very small magnetic fields can be used. Magnetic force microscopy or handheld magnetic readers can also be used in some cases. Another suitable technique is magnetic resonance microscopy.

In some embodiments, the particles may include fluorescent or luminescent particles, particles that include fluorescent or luminescent moieties, and/or plasmon resonant particles. In certain embodiments, the particles have detectable optical and/or magnetic properties. For example, the particles may include one or more diagnostic agents, or the particles may themselves be detectable in some fashion. For instance, in some cases, an optically detectable particle can be detected within a living cell, or within tissue. Optical detection can be accomplished by any suitable technique, for example, detecting the scattering, emission, and/or absorption of light that falls within the optical region of the spectrum, e.g., that portion of the spectrum extending from approximately 180 nm to several microns, for example, between 380 nm and 750 nm.

In certain embodiments, the particles include quantum dots (QDs). QDs are bright, fluorescent nanocrystals with physical dimensions small enough such that the effect of quantum confinement gives rise to unique optical and electronic properties. Semiconductor QDs are often composed of atoms from groups II-VI or III-V in the periodic table, but other compositions are possible, for example, gold quantum dots. By varying their size and composition, the emission wavelength can be tuned (i.e., adjusted in a predictable and controllable manner) from the blue to the near infrared. QDs generally have a broad absorption spectrum and a narrow emission spectrum. Thus different QDs having distinguishable optical properties (e.g., peak emission wavelength) can be excited using a single source.

Any suitable technique may be used to associate or link the various portions of an activation or a receiving composition with each other. For example, in one set of embodiments, the portions may be physically and/or chemically associated with each other, for instance using covalent bonds or non-covalent interactions such as ionic interactions, van der Waals forces, hydrophobic interactions, or the like. For instance, a composition may comprise various combinations of a particle, a liposome, a targeting entity, a pharmaceutical agent, a diagnostic agent, a modulating entity, or an agent able to bind a product of a biological cascade, and some or all of these portions may be associated directly with other and/or associated via one or more linkers.

Linkers may be selected from any suitable class of compounds and may comprise polymers or copolymers of organic acids, aldehydes, alcohols, thiols, amines and the like. For example, polymers or copolymers of hydroxy-, amino-, or di-carboxylic acids, such as glycolic acid, lactic acid, sebacic acid, or sarcosine may be employed. Alternatively, polymers or copolymers of saturated or unsaturated hydrocarbons such as ethylene glycol, propylene glycol, saccharides, and the like may be employed. In some cases, the linker is of an appropriate length that allows the binding element, which is to be attached, to interact freely with molecules in a sample solution and to form effective binding. The linker, in some embodiments, comprises at least two reactive groups able to react with each other. The two reactive groups may be of the same or different chemical moiety. Examples of chemical moieties include maleimide or vinyl sulfone.

Methods for binding a linker will vary. For example, siloxane bonds may be formed via reactions between the trichlorosilyl or trisalkoxy groups of a linker and the hydroxyl groups on the support surface. The linkers may be either branched or unbranched, but this and other structural attributes of the linker should not interfere stereochemically with relevant functions of the binding elements, such as a ligand-antiligand interaction. Protection groups, known to those skilled in the art, may be used to prevent the linker end groups from undesired or premature reactions. For instance, U.S. Pat. No. 5,412,087 describes the use of photo-removable protection groups on a linker thiol group.

As a specific example, in one set of embodiments, two or more portions may be associated via a thermally responsive linker, which can mediate their association in a temperature-sensitive manner. For instance, a composition may comprise a particle and an agent to be delivered to the subject (for example, a targeting entity, a diagnostic agent, a therapeutic agent, a modulating entity, etc.), and the thermally responsive linker may mediate their association in a temperature-sensitive manner, for example, by causing dissociation of the two when the temperature meets or exceeds a trigger temperature. As a specific example, a composition may comprise a particle and a pharmaceutical agent linked by a thermally-responsive linker, and when the composition is exposed to a trigger temperature and/or a temperature higher than the trigger temperature, the thermally-responsive linker may be no longer capable of mediating their association (i.e. the thermally-responsive linker is “disrupted”), and the pharmaceutical agent may be released from the particle. In some embodiments, thermally-responsive linkers are able to mediate the association of a composition for which disruption of the composition results in release of a portion of the composition. In some embodiments, this may allow the enhancement of transport or clearance of portions of the composition. For example, the composition may be too large for clearance in the body, but the individual portions may be small enough for clearance in the body.

In some embodiments, disruption of the linker occurs at temperatures higher than ambient temperature. In some embodiments, disruption of the linker occurs at temperatures higher than body temperature. The thermally-responsive linkers may be modulated such that the linker dissociates at different trigger temperatures. Such modulation enables production of thermally-responsive linkers having a specific and/or desired trigger temperature and enables multiplexing of several different drug release schemes. Any substance that is responsive to changes in temperature (e.g., displays different properties at different temperatures) may be used a thermally-responsive linker. In some embodiments, thermally-responsive linkers include nucleic acids, peptides and/or proteins, carbohydrates, polymers, or the like. In some embodiments, thermally-responsive linkers are hybrid linkers. The term “hybrid linkers” refers to thermally-responsive linkers including at least two of the following: nucleic acids, proteins/peptides, carbohydrates, lipids, polymers, and small molecules.

As mentioned, in some embodiments, the composition may contain more than one thermally-responsive linker. For example, multiple thermally-responsive linkers may be used to cause the release of more than one portion of the composition. In some embodiments, the multiple different thermally-responsive linkers are sensitive to different temperatures, which may be used to deliver different portions of the composition at different points in time. For example, the composition may release multiple pharmaceutical agents at different times, e.g., on a dosage schedule. The thermally-responsive linkers may be modulated such that the linker dissociates at different trigger temperatures, enabling multiplexing of several different release schemes. For example, the nucleotide content of a nucleic acid thermally-responsive linker may be modified such that a set of linkers is generated, in which each member of the set is characterized by a different nucleotide content (e.g., nucleotide sequence) and, consequently, a different trigger temperature.

In certain embodiments, thermally-responsive linkers include interactions between complimentary peptides, lipids, polymers, and/or carbohydrates. In certain embodiments, thermally-responsive linkers include proteins which can undergo temperature dependent conformational changes. A thermally-responsive linker can also include, in some cases, any material that swells and/or shrinks in response to temperature changes. In certain embodiments, a thermally-responsive linker includes any material that swells and/or shrinks in response to temperature changes and also that does not break in response to temperature changes. For example, such a thermally-responsive linker may include a polymer such as pNIPAM.

In some embodiments, a thermally-responsive linker includes a nucleic acid. In certain embodiments, thermally-responsive linkers include complementary Watson-Crick base pairing of nucleic acid strands (e.g., DNA, RNA, and/or PNA strands). The thermally-responsive linker may comprise nucleic acids whose properties result from the three-dimensional structure of the nucleic acid (e.g., an aptamer). In some instances, the trigger temperature can be modulated by varying the number of complimentary hybridizing bases on two or more nucleic acid strands. The duplex region may not include any nucleotide mismatches, or the duplex region may be interrupted by 1, 2, 3, 4, 5, or more nucleotide mismatches. The nucleotide mismatches may be contiguous (i.e. mismatches are adjacent to one another) or non-contiguous (i.e. mismatches are separated by one or more base pairs). The presence of mismatches can, in some cases, decrease the trigger temperature relative to the absence of mismatches.

In some embodiments, a thermally-responsive linker includes a duplex region and at least one single-stranded nucleic acid overhang on either side or both sides of the duplex region. The trigger temperature can be modulated by varying the nucleotide content of the nucleic acid strands. For example, increasing the amount of guanine and/or cytosine relative to the amount of adenine, thymine, and/or uracil tends to raise the trigger temperature of a thermally-responsive linker. Likewise, increasing the amount of adenine, thymine, and/or uracil relative to the amount of guanine and/or cytosine tends to lower the trigger temperature of a thermally-responsive linker. In some cases, the trigger temperature can be modulated by including one or more modified nucleotide residues.

In some embodiments, a thermally-responsive linker includes one or more amino acids. For instance, the link may be a protein and/or peptide linker that includes two or more moieties that interact with one another in a heat-sensitive manner. Protein-based interactions may be heat-sensitive if their association is at least partially-mediated by hydrogen bonding. In certain cases, thermally-responsive linkers include any protein-protein interaction domains that involve hydrogen bonding. In certain embodiments, thermally-responsive linkers are based on coil geometries (e.g., alpha helices, leucine zippers, collagen helices, etc.), beta sheet motifs (e.g., amphiphilic peptides), etc. The protein and/or peptide linkers may include any heat-sensitive affinity interaction, ligand-receptor interactions (e.g., TGF alpha-EGF receptor interactions), antibody-antigen interactions, other types of affinity interactions (e.g., any two proteins which specifically bind to one another), ligand-receptor interactions, enzyme-substrate interactions, or other types of affinity interaction (e.g., an interaction between any proteins which specifically bind to one another).

In some embodiments, the thermally-responsive linker includes a polymer (e.g., a synthetic polymer). For instance, the thermally-responsive linker may include sol-gel hydrogels whose transition is based on temperature, including natural polymers, poly(ethylene oxide)/poly (propylene oxide) block copolymers, N-isopropylacrylamide copolymers, etc. In some embodiments, polymer-based thermally-responsive linkers include multiphase hydrogels.

In some embodiments, a thermally-responsive linker may include at least one individual component which has a temperature-sensitive three-dimensional conformation. In certain embodiments, thermally-responsive linkers include proteins and/or peptides which can undergo temperature-dependent conformational changes. In certain cases, protein and/or peptide structures containing hydrogen bonds (e.g., alpha helices, beta sheets, amphiphilic peptides, etc.) encapsulate hydrophobic agents in the interior of the structures and, upon disassociation (e.g., upon exposure to a trigger temperature), release portions of the composition. Release can occur in some cases because the composition is no longer able to contain a portion therein (e.g., the portion can “leak out” of the protein and/or peptide structure).

According to certain aspects, the systems and methods discussed herein can be applied to a variety of different classes of diseases, disorders and conditions can be treated and/or diagnosed. For example, a disease may include a tumor, including benign, pre-malignant and/or malignant tumors. Cancer, which includes malignant tumors, is a class of diseases in which a group of cells display uncontrolled growth, invasion, and sometimes metastasis. As another example, various inflammatory diseases can be treated and/or diagnosed using the systems and methods discussed herein. There are many conditions and diseases that are linked to chronic inflammation or that have an inflammatory component, such as Alzheimer's disease, cancer, Parkinson's disease, diabetes mellitus, atherosclerosis, cataract, reperfusion injury, infectious meningitis, rheumatoid arthritis, asthma, sepsis, inflammatory bowel disease and multiple sclerosis, etc.

The systems and methods discussed herein may be used in some embodiments to alter or affect the delivery of one or more therapeutic, diagnostic, and/or prophylactic agents to specific tissues, cells, and/or subcellular locales. For example, a selected site may be treated by delivering a receiving composition and/or an activation composition systemically and/or locally to the selected site.

In certain embodiments, a selected site includes cancer cells, and the receiving composition and/or the activation composition may include one or more anti-cancer agents. In certain embodiments, the selected site is a tumor, and nearby blood vessels, and the receiving composition and/or the activation composition include a vascular disruption agent. Vascular disruption is a therapeutic strategy that includes depriving tumors of blood supply. In solid tumors, vascular disruption agents, such as ZYBRESTAT (combretastatin A4 phosphate) and OXi4503 (the diphosphate prodrug of combretastatin A1) (licensed and developed by Oxigene), can cause tumor cell death by rapidly reducing blood-flow to the tumor, thereby depriving it of oxygen and nutrients used for survival. The receiving composition and/or the activation composition can be delivered simultaneously or sequentially, in any suitable order. In some embodiments, an activation composition may be delivered to a selected site, to cause at least a portion of the selected site to change from a first condition to a second condition. For example, the activation composition may cause a selected site to coagulate, to change its permeability, to increase in temperature, to inflame, etc.

These changes may enhance the delivery and/or effectiveness of a receiving composition, for example, for treatment and/or a diagnosis of the selected site. For example, the activation composition may allow heating of a selected site (e.g., by RF ablation and other techniques described herein), or the activation composition may be used to deliver a tumor-targeted tissue factor protein and/or a material that is activatable as a heat source (e.g., gold nanorods) and subsequently heating (e.g., photothermally) the heat source to stimulate a series of effects for recruiting the receiving composition. For example, heating can dilate tumor blood vessels and transvascular pores, and/or increase tumor perfusion and vascular escape of circulating materials (such as nanoparticles). Vessel dilation can also stimulate a clotting cascade. These effects may help to recruit the receiving composition (e.g., doxorubicin-loaded micelles that sense tumor heating by binding to regions of coagulation; clot-targeted, drug-carrying micelles, and/or thermally-labile second components that can release a therapeutic agent) to the selected site. In some embodiments, changing a selected site can catalyze the production of intravascular binding sites that can be accessible to drug-carrying particles still in circulation, e.g., to destroy the selected site. In addition, heating can in some cases provide local and specific tumor ablation.

As another example, the activation composition can include permeability-enhancing materials (e.g., nanoparticles) to locally dilate tumor vessel pores and amplify the targeting of the receiving composition (e.g., therapeutic and/or diagnostic nanoparticles). The receiving composition (e.g., clinically approved formulations such as Doxil and Abraxane and/or imaging agents) can then benefit from increased access to tumor cells and be more tumor targeted. For example, the activation composition and the receiving composition may be related using molecular pathways in which the activation composition up-regulates or reveals a receptor for the receiving composition to amplify targeting.

Another aspect of the invention provides a method of administering a composition of the invention to a subject. When administered, the compositions of the invention are applied in a therapeutically effective, pharmaceutically acceptable amount as a pharmaceutically acceptable formulation. As used herein, the term “pharmaceutically acceptable” is given its ordinary meaning as used in the art. Pharmaceutically acceptable compounds are generally compatible with other materials of the formulation and are not generally deleterious to the subject. A composition of the invention (or prodrug form of the composition) may be administered to the subject in any therapeutically effective dose or treatment. A therapeutically effective amount may be determined by those of ordinary skill in the art, for instance, employing factors such as those further described below and using no more than routine experimentation.

In administering the compositions of the invention to a subject, dosing amounts, dosing schedules, routes of administration, and the like may be selected so as to affect known activities of the compositions of the invention. Dosages may be estimated based on the results of experimental models, optionally in combination with the results of assays of compositions of the present invention. Dosage may be adjusted appropriately to achieve desired drug levels, local or systemic, depending upon the mode of administration. The doses may be given in one or several administrations per day. In some cases, parenteral administration of the composition may be from one to several orders of magnitude lower dose per day, as compared to oral doses. In the event that the response of a particular subject is insufficient at such doses, even higher doses (or effectively higher doses by a different, more localized delivery route) may be employed to the extent that subject tolerance permits. Multiple doses per day are also contemplated, in certain cases, to achieve appropriate levels of the composition within the subject or within the active site of the subject, such as within the brain.

The dose of the composition to the subject may be such that a therapeutically effective amount of the composition (or a portion thereof) reaches or enters the selected site. The dosage may be given in some cases at the maximum amount while avoiding or minimizing any potentially detrimental side effects to the subject. The dosage of the composition that is actually administered is dependent upon factors such as the final concentration desired at the active site, the method of administration to the subject, the efficacy of the composition, the longevity (i.e., half-life) within the subject of the composition, the timing of administration relative to the formation of the tangles and/or plaques the frequency of treatment, the effect of concurrent treatments, etc. The dose delivered may also depend on conditions associated with the subject, and can vary from subject to subject in some cases. For example, the age, sex, weight, size, environment, physical conditions, or current state of health of the subject may also influence the dose required and/or the concentration of the composition (or portion thereof) at the active site. Variations in dosing may occur between different individuals or even within the same individual on different days. It may be preferred that a maximum dose be used, that is, the highest safe dose according to sound medical judgment. Preferably, the dosage form is such that it does not substantially deleteriously affect the subject. The specific dosage(s) given to the subject can thus be determined by those of ordinary skill in the art, using no more than routine experimentation.

Administration of the compositions of the invention may be accomplished by any medically acceptable method which allows the composition (or portion thereof) to reach its target. The particular mode selected will depend, of course, upon factors such as the particular composition, the severity of the state of the subject being treated, or the dosage required for therapeutic efficacy. As used herein, a “medically acceptable” mode of treatment is a mode able to produce effective levels of the composition (or portion thereof) within the subject, without causing clinically unacceptable adverse effects. The location may be a selected site where a composition (or portion thereof) of the invention is able to act.

Any medically acceptable method may be used to administer the composition to the subject. The administration may be localized (i.e., to a particular region, physiological system, tissue, organ, or cell type) or systemic, depending on the condition to be treated. For example, the composition may be administered orally, vaginally, rectally, buccally, pulmonary, topically, nasally, transdermally through parenteral injection or implantation, via surgical administration, or any other method of administration where access to the target by the composition of the invention is achieved. Examples of parenteral modalities that can be used with the invention include intravenous, intradermal, subcutaneous, intracavity, intramuscular, intraperitoneal, epidural, or intrathecal. Examples of implantation modalities include any implantable or injectable drug delivery system. In certain cases, the article may be removed (and optionally replaced with a new article) after a suitable period of time has passed, for example, after a fixed time, such as a day, a week, or a month. In certain instances, the article may be removed when a certain condition is reached. In other cases, the article may be allowed to remain within the subject indefinitely. For example, a sponge may include one or more compositions of the invention, which may be released from the sponge upon implantation, or which may remain mobilized within the sponge.

Oral administration may be preferred in some embodiments because of the convenience to the subject as well as the dosing schedule. Compositions suitable for oral administration may be presented as discrete units such as hard or soft capsules, pills, cachettes, tablets, troches, or lozenges, each containing a predetermined amount of the active compound of the composition. Other oral compositions suitable for use with the invention include solutions or suspensions in aqueous or non-aqueous liquids such as a syrup, an elixir, or an emulsion. In another set of embodiments, the composition may be used to fortify a food or a beverage.

In certain embodiments of the invention, the administration of the composition of the invention may be designed so as to result in sequential exposures to the composition over a certain time period, for example, hours, days, weeks, months or years. This may be accomplished by repeated administrations of the composition by one of the methods described above, or by a sustained or controlled release delivery system in which the composition is delivered over a prolonged period without repeated administrations. Administration of the composition using such a delivery system may be, for example, by oral dosage forms, bolus injections, transdermal patches or subcutaneous implants. Maintaining a substantially constant concentration of the composition may be preferred in some cases. However, avoidance of short-term elevated levels of compositions within the body may be desired in some cases, for instance, to minimize the precipitation of silicate kidney stones in the presence of non-physiological levels of silicon and silicon-containing compounds.

Other delivery systems suitable for use with the present invention (e.g., where alteration and/or control of the release kinetics is desired) include time-release, delayed release, sustained release, or controlled release delivery systems. Such systems may avoid repeated administrations of the composition in many cases, increasing convenience to the subject. Many types of release delivery systems are available and known to those of ordinary skill in the art. They include, for example, polymer-based systems such as polylactic and/or polyglycolic acids, polyanhydrides, polycaprolactones and/or combinations of these; nonpolymer systems that are lipid-based including sterols such as cholesterol, cholesterol esters, and fatty acids or neutral fats such as mono-, di- and triglycerides; hydrogel release systems; liposome-based systems; phospholipid based-systems; silastic systems; peptide based systems; wax coatings; compressed tablets using conventional binders and excipients; or partially fused implants. Specific examples include, but are not limited to, erosional systems in which the composition is contained in a form within a matrix (for example, as described in U.S. Pat. Nos. 4,452,775, 4,675,189, and 5,736,152), or diffusional systems in which an active component controls the release rate (for example, as described in U.S. Pat. Nos. 3,854,480, 5,133,974 and 5,407,686). The formulation may be as, for example, microspheres, hydrogels, polymeric reservoirs, cholesterol matrices, or polymeric systems. In some embodiments, the system may allow sustained or controlled release of the composition to occur, for example, through control of the diffusion or erosion/degradation rate of the formulation containing the composition. In addition, a pump-based hardware delivery system may be used to deliver one or more embodiments of the invention.

Use of a long-term release implant may be particularly suitable in some embodiments of the invention. “Long-term release,” as used herein, means that the implant containing the composition is constructed and arranged to deliver therapeutically effective levels of the composition for at least 30 or 45 days, and preferably at least 60 or 90 days, or even longer in some cases. Long-term release implants are well known to those of ordinary skill in the art, and include some of the release systems described above.

Administration of the compositions of the invention (or prodrug form of the composition) can be alone, or in combination with other therapeutic agents and/or compositions. In certain embodiments, the compositions of the invention can be combined with a suitable pharmaceutically acceptable carrier, for example, as incorporated into a liposome, incorporated into a polymer release system, or suspended in a liquid, e.g., in a dissolved form or a colloidal form. The carrier may be either soluble or insoluble, depending on the application. Compositions of the invention that may be pharmaceutically acceptable include not only the active compound, but also formulation ingredients such as salts, carriers, buffering agents, emulsifiers, diluents, excipients, chelating agents, drying agents, antioxidants, antimicrobials, preservatives, binding agents, bulking agents, solubilizers, or stabilizers that may be used with the active compound. For example, if the formulation is a liquid, the carrier may be a solvent, partial solvent, or non-solvent, and may be aqueous or organically based. Examples of suitable formulation ingredients include diluents such as calcium carbonate, sodium carbonate, lactose, kaolin, calcium phosphate, or sodium phosphate; granulating and disintegrating agents such as corn starch or algenic acid; binding agents such as starch, gelatin or acacia; lubricating agents such as magnesium stearate, stearic acid, or talc; time-delay materials such as glycerol monostearate or glycerol distearate; suspending agents such as sodium carboxymethylcellulose, methylcellulose, hydroxypropylmethylcellulose, sodium alginate, polyvinylpyrrolidone; dispersing or wetting agents such as lecithin or other naturally-occurring phosphatides; thickening agents such as cetyl alcohol or beeswax; buffering agents such as acetic acid and salts thereof, citric acid and salts thereof, boric acid and salts thereof, or phosphoric acid and salts thereof; or preservatives such as benzalkonium chloride, chlorobutanol, parabens, or thimerosal. Suitable carrier concentrations can be determined by those of ordinary skill in the art, using no more than routine experimentation. The compositions of the invention may be formulated into preparations in solid, semi-solid, liquid or gaseous forms such as tablets, capsules, elixirs, powders, granules, ointments, solutions, depositories, inhalants or injectables. Those of ordinary skill in the art will know of other suitable formulation ingredients, or will be able to ascertain such, using only routine experimentation.

In general, pharmaceutically acceptable carriers suitable for use in the invention are well-known to those of ordinary skill in the art. As used herein, a “pharmaceutically acceptable carrier” refers to a non-toxic material that does not significantly interfere with the effectiveness of the biological activity of the active compound(s) to be administered, but is used as a formulation ingredient, for example, to stabilize or protect the active compound(s) within the composition before use. The term “carrier” denotes an organic or inorganic ingredient, which may be natural or synthetic, with which one or more active compounds of the invention are combined to facilitate the application of the composition. The carrier may be co-mingled or otherwise mixed with one or more active compounds of the present invention, and with each other, in a manner such that there is no interaction which would substantially impair the desired pharmaceutical efficacy. Pharmaceutically acceptable carriers include, for example, diluents, emulsifiers, fillers, salts, buffers, excipients, drying agents, antioxidants, preservatives, binding agents, bulking agents, chelating agents, stabilizers, solubilizers, silicas, and other materials well-known in the art.

Preparations include sterile aqueous or nonaqueous solutions, suspensions and emulsions, which can be isotonic with the blood of the subject in certain embodiments. Examples of nonaqueous solvents are polypropylene glycol, polyethylene glycol, vegetable oil such as olive oil, sesame oil, coconut oil, peanut oil, injectable organic esters such as ethyl oleate, or fixed oils including synthetic mono or di-glycerides. Aqueous carriers include water, alcoholic/aqueous solutions, emulsions or suspensions, including saline and buffered media. Parenteral vehicles include sodium chloride solution, 1,3-butandiol, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and/or other additives may also be present such as, for example, antimicrobials, antioxidants, chelating agents and inert gases and the like. Those of skill in the art can readily determine the various parameters for preparing and formulating the compositions of the invention without resort to undue experimentation.

In some embodiments, the present invention includes a step of bringing a composition or compound of the invention into association or contact with a suitable carrier, which may constitute one or more accessory ingredients. The final compositions may be prepared by any suitable technique, for example, by uniformly and intimately bringing the composition into association with a liquid carrier, a finely divided solid carrier or both, optionally with one or more formulation ingredients as previously described, and then, if necessary, shaping the product.

In some embodiments, a compound of the present invention may be present as a pharmaceutically acceptable salt. The term “pharmaceutically acceptable salts” includes salts of the compound, prepared in combination with, for example, acids or bases, depending on the particular compounds found within the composition and the treatment modality desired. Pharmaceutically acceptable salts can be prepared as alkaline metal salts, such as lithium, sodium, or potassium salts; or as alkaline earth salts, such as beryllium, magnesium, or calcium salts. Examples of suitable bases that may be used to form salts include ammonium, or mineral bases such as sodium hydroxide, lithium hydroxide, potassium hydroxide, calcium hydroxide, magnesium hydroxide, and the like. Examples of suitable acids that may be used to form salts include inorganic or mineral acids such as hydrochloric, hydrobromic, hydroiodic, hydrofluoric, nitric, carbonic, monohydrogencarbonic, phosphoric, monohydrogenphosphoric, dihydrogenphosphoric, sulfuric, monohydrogensulfuric, phosphorous acids and the like. Other suitable acids include organic acids, for example, acetic, propionic, isobutyric, maleic, malonic, benzoic, succinic, suberic, fumaric, mandelic, phthalic, benzenesulfonic, p-tolylsulfonic, citric, tartaric, methanesulfonic, glucuronic, galacturonic, salicylic, formic, naphthalene-2-sulfonic, and the like. Still other suitable acids include amino acids such as arginate, aspartate, glutamate, and the like.

A variety of definitions are now provided which will aid in understanding various aspects of the invention. Following, and interspersed with these definitions, is further disclosure that will more fully describe the invention.

Associated with: As used herein, the terms “associated with,” “conjugated,” “linked,” “attached,” and “tethered,” when used with respect to two or more moieties, means that the moieties are physically associated or connected with one another, either directly or via one or more additional moieties that serves to link the moieties together, to form a structure that is sufficiently stable so that the moieties remain physically associated (e.g., loaded by doping and encapsulation) under the conditions in which structure is used, e.g., physiological conditions. In some embodiments, the moieties are attached to one another by one or more covalent bonds. In certain instances, the moieties are attached to one another by a mechanism that involves specific (but non-covalent) binding (e.g., streptavidin/avidin interactions, antibody/antigen interactions, ionic interactions, van der Waals forces, hydrophobic interactions, etc.) in addition to and/or instead of covalent bonding interactions. In some embodiments, a sufficient number of weaker interactions (e.g., absorption or other non-covalent bonds) can provide sufficient stability for moieties to remain associated.

Biomolecules: As used herein, the term “biomolecules,” refers to molecules (e.g., proteins, amino acids, peptides, polynucleotides, nucleotides, carbohydrates, sugars, lipids, nucleoproteins, glycoproteins, lipoproteins, steroids, etc.) whether naturally-occurring or artificially created (e.g., by synthetic or recombinant methods) that are commonly found in cells and tissues. Specific classes of biomolecules include, but are not limited to, enzymes, receptors, neurotransmitters, hormones, cytokines, cell response modifiers such as growth factors and chemotactic factors, antibodies, vaccines, haptens, toxins, interferons, ribozymes, anti-sense agents, plasmids, DNA, and RNA.

Nucleic acid: As used herein, the term “nucleic acid,” in its broadest sense, refers to any compound and/or substance that is or can be incorporated covalently, for example, via a phosphodiester linkage, into an oligonucleotide chain and which contains complementary “bases” attached to a polymeric backbone, where the bases are complementary to cytosine, guanine, adenine, thymine, and/or uracil bases on DNA and/or RNA. In some embodiments, a nucleic acid refers to individual nucleic acid residues (e.g. nucleotides and/or nucleosides). In other embodiments, “nucleic acid” refers to an oligonucleotide chain comprising individual nucleic acid residues. As used herein, the terms “oligonucleotide” and “polynucleotide” can be used interchangeably. In some embodiments, “nucleic acid” encompasses RNA as well as single and/or double-stranded DNA and/or cDNA. Furthermore, the terms “nucleic acid,” “DNA,” “RNA,” and/or similar terms include nucleic acid analogs, i.e. analogs having other than a phosphodiester backbone. For example, the so-called “peptide nucleic acids,” which are known in the art and have peptide bonds instead of phosphodiester bonds in the backbone, are considered within the scope of the present disclosure.

The term “nucleotide sequence encoding an amino acid sequence” includes all nucleotide sequences that are degenerate versions of each other and/or encode the same amino acid sequence. Nucleotide sequences that encode proteins and/or RNA may include introns. Nucleic acids can be purified from natural sources, produced using recombinant expression systems and optionally purified, chemically synthesized, etc. Where appropriate, e.g. in the case of chemically synthesized molecules, nucleic acids can comprise nucleoside analogs such as analogs having chemically modified bases or sugars, backbone modifications, etc. A nucleic acid sequence is presented in the 5′ to 3′ direction unless otherwise indicated.

The term “nucleic acid segment” is used herein to refer to a nucleic acid sequence that is a portion of a longer nucleic acid sequence. In some embodiments, a nucleic acid segment comprises at least 3, 4, 5, 6, 7, 8, 9, 10, or more residues. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages). In some embodiments, “unmodified nucleic acids,” meaning nucleic acids (e.g. polynucleotides and residues, including nucleotides and/or nucleosides) that have not been chemically modified in order to facilitate or achieve delivery, are included.

Small molecule: As used herein, the term “small molecule” is used to refer to molecules, whether naturally-occurring or artificially created (e.g., via chemical synthesis), that have a relatively low molecular weight. In general, a “small molecule” is understood in the art to be an organic molecule that is less than about 5 kilodaltons (kDa) in molecular weight. In some embodiments, the small molecule is less than about 4 kDa, less than about 3 kDa, less than about 2 kDa, less than about 1.5 kDa, or less than about 1 kDa. In some embodiments, the small molecule is less than about 800 daltons (D or Da), less than about 600 Da, less than about 500 Da, less than about 400 Da, less than about 300 Da, less than about 200 Da, or less than about 100 Da. In some embodiments, small molecules are non-polymeric. In some embodiments, small molecules are not proteins, peptides, or amino acids. In some embodiments, small molecules are not nucleic acids or nucleotides. In some embodiments, small molecules are not saccharides or polysaccharides. Some small molecules are biologically active in that they produce a local or systemic effect in animals, for example, mammals such as humans. In certain embodiments, the small molecule is a drug. In certain embodiments, the drug is one that has already been deemed safe and effective for use by an appropriate governmental agency or body.

Amino acid: As used herein, term “amino acid,” in its broadest sense, refers to any compound and/or substance that can be incorporated into a polypeptide chain. In some embodiments, an amino acid has the general structure H₂N—C(H)(R)—COOH. In some embodiments, an amino acid is a naturally-occurring amino acid. In some embodiments, an amino acid is a synthetic amino acid; in some embodiments, an amino acid includes a D-amino acid; in some embodiments, an amino acid includes an L-amino acid. “Standard amino acid” refers to any of the twenty standard L-amino acids commonly found in naturally occurring peptides). “Nonstandard amino acid” refers to any amino acid, other than the standard amino acids, regardless of whether it is prepared synthetically or obtained from a natural source. As used herein, “synthetic amino acid” encompasses chemically modified amino acids, including but not limited to salts, amino acid derivatives (such as amides), and/or substitutions. Amino acids, including carboxy- and/or amino-terminal amino acids in peptides, can be modified by methylation, amidation, acetylation, and/or substitution with other chemical groups that can change the peptide's circulating half-life without adversely affecting their activity. Some amino acids may also participate in disulfide bonds. The term “amino acid” is used interchangeably with “amino acid residue,” and may refer to a free amino acid and/or to an amino acid residue of a peptide. It will be apparent from the context in which the term is used whether it refers to a free amino acid or a residue of a peptide.

Protein: As used herein, the term “protein” refers to a polypeptide (i.e., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a characteristic portion thereof. Those of ordinary skill will appreciate that a protein can sometimes include more than one polypeptide chain, for example linked by one or more disulfide bonds or associated by other means. Polypeptides may contain L-amino acids, D-amino acids, or both and may contain any of a variety of amino acid modifications or analogs known in the art. Useful modifications include, e.g., terminal acetylation, amidation, lipidation, phosphorylation, glycosylation, acylation, farnesylation, sulfation, etc. In some embodiments, proteins may comprise natural amino acids, non-natural amino acids, synthetic amino acids, and combinations thereof. Peptides from panels of peptides containing random sequences and/or sequences that have been varied consistently to provide a maximally diverse panel of peptides may be used. The terms “polypeptide” and “peptide” are generally used interchangeably herein, and generally refer to a polypeptide having a length of less than about 100 amino acids, or less than 50 amino acids in some cases. Various changes in the amino acid sequence of a peptide can also be made without substantially affecting the function of the peptide. For example, 1, 2, 3, or more such changes such as deletions, insertions, substitutions, etc. may be made. The resulting peptide can have at least 80% sequence identity, e.g., 90% sequence identity, with the original peptide.

Antibody: As used herein, the term “antibody” refers to any immunoglobulin, whether natural or wholly or partially synthetically produced, and to derivatives thereof and characteristic portions thereof. All derivatives thereof which maintain specific binding ability are also included in the term. The term is also intended to include antibody fragments, characteristic portions of antibodies, single chain antibodies, etc. Synthetic binding proteins such as antibodies, etc., can be used. The term also covers any protein having a binding domain which is homologous or largely homologous to an immunoglobulin binding domain. Such proteins may be derived from natural sources, or partly or wholly synthetically produced. An antibody may be monoclonal or polyclonal. An antibody may be a member of any immunoglobulin class, including but not limited to any of the human classes: IgG, IgM, IgA, IgD, and IgE. As used herein, the terms “antibody fragment” or “characteristic portion of an antibody” are used interchangeably and refer to any derivative of an antibody which is less than full-length. In general, an antibody fragment retains at least a significant portion of the full-length antibody's specific binding ability. Examples of antibody fragments include, but are not limited to, Fab, Fab′, F(ab′)₂, scFv, Fv, dsFv diabody, and Fd fragments. In some embodiments, antibodies may have reduced effect or functions and/or bispecific molecules. In some embodiments, antibodies may include Fab fragments and/or fragments produced by a Fab expression library. An antibody fragment may be produced by any technique. For example, an antibody fragment may be enzymatically or chemically produced by fragmentation of an intact antibody, and/or it may be recombinantly produced from a gene encoding the partial antibody sequence. Alternatively or additionally, an antibody fragment may be wholly or partially synthetically produced. An antibody fragment may optionally comprise a single chain antibody fragment. Alternatively or additionally, an antibody fragment may comprise multiple chains which are linked together, for example, by disulfide linkages. An antibody fragment may optionally comprise a multimolecular complex. A functional antibody fragment typically comprises at least about 50 amino acids and more typically comprises least about 200 amino acids. In some embodiments, antibodies include chimeric (e.g., “humanized”) and single chain (recombinant) antibodies. In some embodiments, antibodies have reduced effector functions and/or bispecific molecules. In some embodiments, antibodies include fragments produced by a Fab expression library.

Carbohydrate: A carbohydrate may be a polysaccharide including simple sugars (or their derivatives) connected by glycosidic bonds. The sugars may include, but are not limited to, glucose, fructose, galactose, ribose, lactose, sucrose, maltose, trehalose, cellobiose, mannose, xylose, arabinose, glucoronic acid, galactoronic acid, mannuronic acid, glucosamine, galatosamine, and neuramic acid. In some embodiments, a carbohydrate includes one or more of pullulan, cellulose, microcrystalline cellulose, hydroxypropyl methylcellulose, hydroxycellulose, methylcellulose, dextran, cyclodextran, glycogen, starch, hydroxyethylstarch, carageenan, glycon, amylose, chitosan, N,O-carboxylmethylchitosan, algin and alginic acid, starch, chitin, heparin, konjac, glucommannan, pustulan, heparin, hyaluronic acid, curdlan, and xanthan. In some embodiments, the carbohydrate is aminated, carboxylated, acetylated and/or sulfated. In some embodiments, hydrophilic polysaccharides are modified to become hydrophobic by introducing a large number of side chain hydrophobic groups.

Fatty acid: A fatty acid typically is a carboxylic acid with a long unbranched aliphatic tail (chain), which may be saturated or unsaturated. For instance, in some embodiments, a fatty acid group includes digestible, long chain (e.g., C₈-C₅₀), and substituted or unsubstituted hydrocarbons. In some embodiments, a fatty acid group includes a C₁₀-C₂₀ fatty acid or salt thereof. In some embodiments, a fatty acid group includes a C₁₅-C₂₀ fatty acid or salt thereof. In some embodiments, a fatty acid group includes a C₁₅-C₂₅ fatty acid or salt thereof. In some embodiments, a fatty acid group is unsaturated. In some embodiments, a fatty acid group is monounsaturated. In some embodiments, a fatty acid group is polyunsaturated. In some embodiments, a double bond of an unsaturated fatty acid group is in the cis conformation. In some embodiments, a double bond of an unsaturated fatty acid is in the trans conformation. In some embodiments, a fatty acid group includes one or more of butyric, caproic, caprylic, capric, lauric, myristic, palmitic, stearic, arachidic, behenic, or lignoceric acid. In some embodiments, a fatty acid group includes one or more of palmitoleic, oleic, vaccenic, linoleic, alpha-linoleic, gamma-linoleic, arachidonic, gadoleic, arachidonic, eicosapentaenoic, docosahexaenoic, or erucic acid.

Prophylactic agent: A prophylactic agent, as used herein, is one that is given to a subject to prevent the occurrence of a disease in the subject. The subject typically does not actively have the disease treated with the prophylactic agent. Prophylactic agents may include antigens of such bacterial organisms as Streptococccus pnuemoniae, Haemophilus influenzae, Staphylococcus aureus, Streptococcus pyrogenes, Corynebacterium diphtheriae, Listeria monocytogenes, Bacillus anthracis, Clostridium tetani, Clostridium botulinum, Clostridium perfringens, Neisseria meningitidis, Neisseria gonorrhoeae, Streptococcus mutans, Pseudomonas aeruginosa, Salmonella typhi, Haemophilus parainfluenzae, Bordetella pertussis, Francisella tularensis, Yersinia pestis, Vibrio cholerae, Legionella pneumophila, Mycobacterium tuberculosis, Mycobacterium leprae, Treponema pallidum, Leptospirosis interrogans, Borrelia burgdorferi, Camphylobacter jejuni, and the like; antigens of such viruses as smallpox, influenza A and B, respiratory syncytial virus, parainfluenza, measles, HIV, varicella-zoster, herpes simplex 1 and 2, cytomegalovirus, Epstein-Ban virus, rotavirus, rhinovirus, adenovirus, papillomavirus, poliovirus, mumps, rabies, rubella, coxsackieviruses, equine encephalitis, Japanese encephalitis, yellow fever, Rift Valley fever, hepatitis A, B, C, D, and E virus, and the like; antigens of fungal, protozoan, and parasitic organisms such as Cryptococcus neoformans, Histoplasma capsulatum, Candida albicans, Candida tropicalis, Nocardia asteroides, Rickettsia ricketsii, Rickettsia typhi, Mycoplasma pneumoniae, Chlamydial psittaci, Chlamydial trachomatis, Plasmodium falciparum, Trypanosoma brucei, Entamoeba histolytica, Toxoplasma gondii, Trichomonas vaginalis, Schistosoma mansoni, and the like.

Biocompatible: As used herein, the term “biocompatible” refers to substances that are not toxic to cells. In some embodiments, a substance is considered to be “biocompatible” if its addition to cells in vivo does not induce substantial inflammation and/or other adverse effects in vivo. In some embodiments, a substance is considered to be “biocompatible” if its addition to cells in vitro or in vivo results in less than or equal to about 50%, about 45%, about 40%, about 35%, about 30%, about 25%, about 20%, about 15%, about 10%, about 5%, or less than about 5% cell death. In some embodiments, a substance is considered to be biocompatible if its addition to cells does not induce adverse effects. It will be recognized, of course, that “biocompatibility” is a relative term, and some degree of inflammatory and/or immune response is to be expected even for substances that are highly compatible with living tissue. However, non-biocompatible materials are typically those substances that are highly inflammatory and/or are acutely rejected by the immune system, i.e., a non-biocompatible substance introduced to a subject may provoke a major immune response. In some cases, the immune response is severe enough that the rejection of the substance by the immune system cannot be adequately controlled, in some cases even with the use of immunosuppressant drugs.

Biodegradable: As used herein, the term “biodegradable” refers to substances that are degraded under physiological conditions. A biodegradable substance is one that undergoes breakdown under physiological conditions over the course of a therapeutically relevant time period (e.g., weeks, months, or years). In some embodiments, a biodegradable substance is a substance that is broken down by cellular machinery. In some embodiments, a biodegradable substance is a substance that is broken down by chemical processes. In some embodiments, the biodegradable substance may be bioabsorbable (or bioresorbable) within the subject. For example, some biodegradable substances may react with water and/or bodily fluids and may dissolve, or at least be partially degraded, e.g., via hydrolytic reactions. Such substances may degrade, for example, through hydrolysis, enzymatic action, bulk erosion, or physical surface erosion, or a combination of the above.

Binding partner: The term “binding partner” refers to a molecule that can undergo binding with a particular molecule, e.g., an analyte. For example, the binding may be highly specific and/or non-covalent. Binding partners which form highly specific, non-covalent, physiochemical interactions with one another are defined herein as “complementary.” Biological binding partners are examples. For example, Protein A is a binding partner of the biological molecule IgG, and vice versa. Other non-limiting examples include nucleic acid-nucleic acid binding, nucleic acid-protein binding, protein-protein binding, enzyme-substrate binding, receptor-ligand binding, receptor-hormone binding, antibody-antigen binding, etc. Binding partners include specific, semi-specific, and non-specific binding partners as known to those of ordinary skill in the art. For example, Protein A is usually regarded as a “non-specific” or semi-specific binder. As another example, an enzyme such as glucose oxidase or glucose 1-dehydrogenase, or a lectin such as concanavalin A may be able to bind to glucose.

As additional examples, binding partners may include antibody/antigen pairs, ligand/receptor pairs, enzyme/substrate pairs and complementary nucleic acids or aptamers. Examples of suitable epitopes which may be used for antibody/antigen binding pairs include, but are not limited to, HA, FLAG, c-Myc, glutatione-5-transferase, His₆, GFP, DIG, biotin and avidin. Antibodies may be monoclonal or polyclonal. Suitable antibodies for use as binding partners include antigen-binding fragments, including separate heavy chains, light chains Fab, Fab′, F(ab′)₂, Fabc, and Fv. Antibodies also include bispecific or bifunctional antibodies. Exemplary binding partners include biotin/avidin, biotin/streptavidin, biotin/neutravidin and glutathione-S-transferase/glutathione.

Binding: The term “binding” generally refers to the interaction between a corresponding pair of molecules or surfaces that exhibit mutual affinity or binding capacity, typically due to specific or non-specific binding or interaction, including, but not limited to, biochemical, physiological, and/or chemical interactions. The binding may be between biological molecules, including proteins, nucleic acids, glycoproteins, carbohydrates, hormones, or the like. Specific non-limiting examples include antibody/antigen, antibody/hapten, enzyme/substrate, enzyme/inhibitor, enzyme/cofactor, binding protein/substrate, carrier protein/substrate, lectin/carbohydrate, receptor/hormone, receptor/effector, complementary strands of nucleic acid, protein/nucleic acid repressor/inducer, ligand/cell surface receptor, virus/ligand, virus/cell surface receptor, etc. As another example, the binding agent may be a chelating agent (e.g., ethylenediaminetetraacetic acid) or an ion selective polymer (e.g., a block copolymer such as poly(carbonate-b-dimethylsiloxane), a crown ether, or the like). As another example, the binding partners may be biotin and streptavidin, or the binding partners may be various antibodies raised against a protein.

Specifically binds: The term “specifically binds,” when referring to a binding partner (e.g., protein, nucleic acid, antibody, etc.), refers to a reaction that is determinative of the presence and/or identity of one or other member of the binding pair in a mixture of heterogeneous molecules (e.g., proteins and other biologics). Thus, for example, in the case of a receptor/ligand binding pair, the ligand would specifically and/or preferentially select its receptor from a complex mixture of molecules, or vice versa. An enzyme would specifically bind to its substrate, a nucleic acid would specifically bind to its complement, an antibody would specifically bind to its antigen, etc. The binding may be by one or more of a variety of mechanisms including, but not limited to ionic interactions or electrostatic interactions, covalent interactions, hydrophobic interactions, van der Waals interactions, etc.

Nanoparticle: As used herein, the term “nanoparticle” refers to any particle having a diameter of less than 1000 nanometers (nm). Nanoparticle can be clusters, nanospheres, nanorods, nanofibers, etc. Metal, dielectric and semiconductor nanoparticles can be used, as well as hybrid structures (e.g., core-shell nanoparticles). Semi-solid and soft nanoparticles can be manufactured. In general, the nanoparticles will have dimensions small enough to allow their uptake by eukaryotic cells. The nanoparticles may have a longest straight dimension (e.g., diameter) of 200 nm or less. In some embodiments, the nanoparticles have a diameter of 100 nm or less. Smaller nanoparticles, e.g. having diameters of 50 nm or less, e.g., 5 nm-30 nm, are used in some embodiments. In some embodiments, nanoparticles can be polymeric particles to deliver and/or release drugs. In some embodiments, nanoparticles can be optically or magnetically detectable. In some embodiments, intrinsically fluorescent or luminescent nanoparticles, nanoparticles that include fluorescent or luminescent moieties, plasmon resonant nanoparticles, and magnetic nanoparticles are among the detectable nanoparticles that are used in various embodiments. In certain embodiments, nanoparticles are quantum dots, i.e., bright, fluorescent nanocrystals with physical dimensions small enough such that the effect of quantum confinement gives rise to unique optical and electronic properties. In certain embodiments, optically detectable nanoparticles are metal nanoparticles. Metals of use in the nanoparticles include, but are not limited to, gold, silver, iron, cobalt, zinc, cadmium, nickel, gadolinium, chromium, copper, manganese, palladium, tin, and alloys and/or oxides thereof. Nanoparticles also includes atomic clusters. In some embodiments, a nanoparticle can have a diameter of approximately 1 nm or less and can contain from several atoms (e.g., 3-4 atoms) up to several hundred atoms (e.g., 300-400 atoms). The nanoparticle may have a variety of different shapes including, for example, spheres, oblate spheroids, cylinders, shells, cubes, pyramids, rods (e.g., cylinders or elongated structures having a square or rectangular cross-section), tetrapods (particles having four leg-like appendages), triangles, and/or prisms.

Magnetic particle: “Magnetic particles” refer to magnetically responsive particles that react to a magnetic force resulting from a magnetic field. In some cases, the magnetic particle may contain one or more metals or oxides or hydroxides thereof. The magnetic field can attract or repel the magnetic particles towards or away from the source of the magnetic field, respectively, optionally causing acceleration or movement in a desired direction in space.

Subject: As discussed herein, the subject is generally a human, although non-human subjects may also be used in certain embodiments of the invention, for instance, other mammals such as a dog, a cat, a horse, a rabbit, a cow, a pig, a sheep, a goat, a rat (e.g., Rattus Norvegicus), a mouse (e.g., Mus musculus), a guinea pig, a hamster, a primate (e.g., a monkey, a chimpanzee, a baboon, an ape, a gorilla, etc.), or the like.

Cancer: The term “cancer,” as used herein, may include, but is not limited to: biliary tract cancer; bladder cancer; brain cancer including glioblastomas and medulloblastomas; breast cancer; cervical cancer; choriocarcinoma; colon cancer; endometrial cancer; esophageal cancer; gastric cancer; hematological neoplasms including acute lymphocytic and myelogenous leukemia; multiple myeloma; AIDS-associated leukemias and adult T-cell leukemia lymphoma; intraepithelial neoplasms including Bowen's disease and Paget's disease; liver cancer; lung cancer; lymphomas including Hodgkin's disease and lymphocytic lymphomas; neuroblastomas; oral cancer including squamous cell carcinoma; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, and osteosarcoma; skin cancer including melanoma, Kaposi's sarcoma, basocellular cancer, and squamous cell cancer; testicular cancer including germinal tumors such as seminoma, non-seminoma, teratomas, choriocarcinomas; stromal tumors and germ cell tumors; thyroid cancer including thyroid adenocarcinoma and medullar carcinoma; and renal cancer including adenocarcinoma and Wilms' tumor. Commonly encountered cancers include breast, prostate, lung, ovarian, colorectal, and brain cancer. In general, an effective amount of the one or more compositions of the invention for treating cancer will be that amount necessary to inhibit mammalian cancer cell proliferation in situ. Those of ordinary skill in the art are well-schooled in the art of evaluating effective amounts of anti-cancer agents.

The term “cancer treatment” as used herein, may include, but is not limited to, chemotherapy, radiotherapy, adjuvant therapy, surgery, or any combination of these and/or other methods. Aspects of cancer treatment may vary, for instance, depending on the subject being treated. Examples include, but are not limited to, dosages, timing of administration, duration of treatment, etc. One of ordinary skill in the medical arts can determine an appropriate cancer treatment for a subject.

Examples of anti-cancer agents and drugs that can be used in combination with one or more compositions of the invention include, but are not limited to, any one or more of 20-epi-1,25 dihydroxyvitamin D3, 4-ipomeanol, 5-ethynyluracil, 9-dihydrotaxol, abiraterone, acivicin, aclarubicin, acodazole hydrochloride, acronine, acylfulvene, adecypenol, adozelesin, aldesleukin, all-tk antagonists, altretamine, ambamustine, ambomycin, ametantrone acetate, amidox, amifostine, aminoglutethimide, aminolevulinic acid, amrubicin, amsacrine, anagrelide, anastrozole, andrographolide, angiogenesis inhibitors, antagonist D, antagonist G, antarelix, anthramycin, anti-dorsalizing morphogenetic protein-1, antiestrogen, antineoplaston, antisense oligonucleotides, aphidicolin glycinate, apoptosis gene modulators, apoptosis regulators, apurinic acid, ARA-CDP-DL-PTBA, arginine deaminase, asparaginase, asperlin, asulacrine, atamestane, atrimustine, axinastatin 1, axinastatin 2, axinastatin 3, azacitidine, azasetron, azatoxin, azatyrosine, azetepa, azotomycin, baccatin III derivatives, balanol, batimastat, benzochlorins, benzodepa, benzoylstaurosporine, beta lactam derivatives, beta-alethine, betaclamycin B, betulinic acid, BFGF inhibitor, bicalutamide, bisantrene, bisantrene hydrochloride, bisaziridinylspermine, bisnafide, bisnafide dimesylate, bistratene A, bizelesin, bleomycin, bleomycin sulfate, BRC/ABL antagonists, breflate, brequinar sodium, bropirimine, budotitane, busulfan, buthionine sulfoximine, cactinomycin, calcipotriol, calphostin C, calusterone, camptothecin derivatives, canarypox IL-2, capecitabine, caracemide, carbetimer, carboplatin, carboxamide-amino-triazole, carboxyamidotriazole, carest M3, carmustine, cam 700, cartilage derived inhibitor, carubicin hydrochloride, carzelesin, casein kinase inhibitors, castanospermine, cecropin B, cedefingol, cetrorelix, chlorambucil, chlorins, chloroquinoxaline sulfonamide, cicaprost, cirolemycin, cisplatin, cis-porphyrin, cladribine, clomifene analogs, clotrimazole, collismycin A, collismycin B, combretastatin A4, combretastatin analog, conagenin, crambescidin 816, crisnatol, crisnatol mesylate, cryptophycin 8, cryptophycin A derivatives, curacin A, cyclopentanthraquinones, cyclophosphamide, cycloplatam, cypemycin, cytarabine, cytarabine ocfosfate, cytolytic factor, cytostatin, dacarbazine, dacliximab, dactinomycin, daunorubicin hydrochloride, decitabine, dehydrodidemnin B, deslorelin, dexifosfamide, dexormaplatin, dexrazoxane, dexverapamil, dezaguanine, dezaguanine mesylate, diaziquone, didemnin B, didox, diethylnorspermine, dihydro-5-azacytidine, dioxamycin, diphenyl spiromustine, docetaxel, docosanol, dolasetron, doxifluridine, doxorubicin, doxorubicin hydrochloride, droloxifene, droloxifene citrate, dromostanolone propionate, dronabinol, duazomycin, duocarmycin SA, ebselen, ecomustine, edatrexate, edelfosine, edrecolomab, eflornithine, eflornithine hydrochloride, elemene, elsamitrucin, emitefur, enloplatin, enpromate, epipropidine, epirubicin, epirubicin hydrochloride, epristeride, erbulozole, erythrocyte gene therapy vector system, esorubicin hydrochloride, estramustine, estramustine analog, estramustine phosphate sodium, estrogen agonists, estrogen antagonists, etanidazole, etoposide, etoposide phosphate, etoprine, exemestane, fadrozole, fadrozole hydrochloride, fazarabine, fenretinide, filgrastim, finasteride, flavopiridol, flezelastine, floxuridine, fluasterone, fludarabine, fludarabine phosphate, fluorodaunorunicin hydrochloride, fluorouracil, fluorocitabine, forfenimex, formestane, fosquidone, fostriecin, fostriecin sodium, fotemustine, gadolinium texaphyrin, gallium nitrate, galocitabine, ganirelix, gelatinase inhibitors, gemcitabine, gemcitabine hydrochloride, glutathione inhibitors, hepsulfam, heregulin, hexamethylene bisacetamide, hydroxyurea, hypericin, ibandronic acid, idarubicin, idarubicin hydrochloride, idoxifene, idramantone, ifosfamide, ilmofosine, ilomastat, imidazoacridones, imiquimod, immunostimulant peptides, insulin-like growth factor-1 receptor inhibitor, interferon agonists, interferon alpha-2A, interferon alpha-2B, interferon alpha-N1, interferon alpha-N3, interferon beta-IA, interferon gamma-IB, interferons, interleukins, iobenguane, iododoxorubicin, iproplatin, irinotecan, irinotecan hydrochloride, iroplact, irsogladine, isobengazole, isohomohalicondrin B, itasetron, jasplakinolide, kahalalide F, lamellarin-N triacetate, lanreotide, lanreotide acetate, leinamycin, lenograstim, lentinan sulfate, leptolstatin, letrozole, leukemia inhibiting factor, leukocyte alpha interferon, leuprolide acetate, leuprolide/estrogen/progesterone, leuprorelin, levamisole, liarozole, liarozole hydrochloride, linear polyamine analog, lipophilic disaccharide peptide, lipophilic platinum compounds, lissoclinamide 7, lobaplatin, lombricine, lometrexol, lometrexol sodium, lomustine, lonidamine, losoxantrone, losoxantrone hydrochloride, lovastatin, loxoribine, lurtotecan, lutetium texaphyrin, lysofylline, lytic peptides, maitansine, mannostatin A, marimastat, masoprocol, maspin, matrilysin inhibitors, matrix metalloproteinase inhibitors, maytansine, mechlorethamine hydrochloride, megestrol acetate, melengestrol acetate, melphalan, menogaril, merbarone, mercaptopurine, meterelin, methioninase, methotrexate, methotrexate sodium, metoclopramide, metoprine, meturedepa, microalgal protein kinase C inhibitors, MIF inhibitor, mifepristone, miltefosine, mirimostim, mismatched double stranded RNA, mitindomide, mitocarcin, mitocromin, mitogillin, mitoguazone, mitolactol, mitomalcin, mitomycin, mitomycin analogs, mitonafide, mitosper, mitotane, mitotoxin fibroblast growth factor-saporin, mitoxantrone, mitoxantrone hydrochloride, mofarotene, molgramostim, monoclonal antibody, human chorionic gonadotrophin, monophosphoryl lipid a/myobacterium cell wall SK, mopidamol, multiple drug resistance gene inhibitor, multiple tumor suppressor 1-based therapy, mustard anticancer agent, mycaperoxide B, mycobacterial cell wall extract, mycophenolic acid, myriaporone, n-acetyldinaline, nafarelin, nagrestip, naloxone/pentazocine, napavin, naphterpin, nartograstim, nedaplatin, nemorubicin, neridronic acid, neutral endopeptidase, nilutamide, nisamycin, nitric oxide modulators, nitroxide antioxidant, nitrullyn, nocodazole, nogalamycin, n-substituted benzamides, O6-benzylguanine, octreotide, okicenone, oligonucleotides, onapristone, ondansetron, oracin, oral cytokine inducer, ormaplatin, osaterone, oxaliplatin, oxaunomycin, oxisuran, paclitaxel, paclitaxel analogs, paclitaxel derivatives, palauamine, palmitoylrhizoxin, pamidronic acid, panaxytriol, panomifene, parabactin, pazelliptine, pegaspargase, peldesine, peliomycin, pentamustine, pentosan polysulfate sodium, pentostatin, pentrozole, peplomycin sulfate, perflubron, perfosfamide, perillyl alcohol, phenazinomycin, phenylacetate, phosphatase inhibitors, picibanil, pilocarpine hydrochloride, pipobroman, piposulfan, pirarubicin, piritrexim, piroxantrone hydrochloride, placetin A, placetin B, plasminogen activator inhibitor, platinum complex, platinum compounds, platinum-triamine complex, plicamycin, plomestane, porfimer sodium, porfiromycin, prednimustine, procarbazine hydrochloride, propyl bis-acridone, prostaglandin J2, prostatic carcinoma antiandrogen, proteasome inhibitors, protein A-based immune modulator, protein kinase C inhibitor, protein tyrosine phosphatase inhibitors, purine nucleoside phosphorylase inhibitors, puromycin, puromycin hydrochloride, purpurins, pyrazofurin, pyrazoloacridine, pyridoxylated hemoglobin polyoxyethylene conjugate, RAF antagonists, raltitrexed, ramosetron, RAS farnesyl protein transferase inhibitors, RAS inhibitors, RAS-GAP inhibitor, retelliptine demethylated, rhenium RE 186 etidronate, rhizoxin, riboprine, ribozymes, RII retinamide, RNAi, rogletimide, rohitukine, romurtide, roquinimex, rubiginone B1, ruboxyl, safingol, safingol hydrochloride, saintopin, sarcnu, sarcophytol A, sargramostim, SDI 1 mimetics, semustine, senescence derived inhibitor 1, sense oligonucleotides, signal transduction inhibitors, signal transduction modulators, simtrazene, single chain antigen binding protein, sizofuran, sobuzoxane, sodium borocaptate, sodium phenylacetate, solverol, somatomedin binding protein, sonermin, sparfosate sodium, sparfosic acid, sparsomycin, spicamycin D, spirogermanium hydrochloride, spiromustine, spiroplatin, splenopentin, spongistatin 1, squalamine, stem cell inhibitor, stem-cell division inhibitors, stipiamide, streptonigrin, streptozocin, stromelysin inhibitors, sulfinosine, sulofenur, superactive vasoactive intestinal peptide antagonist, suradista, suramin, swainsonine, synthetic glycosaminoglycans, talisomycin, tallimustine, tamoxifen methiodide, tauromustine, tazarotene, tecogalan sodium, tegafur, tellurapyrylium, telomerase inhibitors, teloxantrone hydrochloride, temoporfin, temozolomide, teniposide, teroxirone, testolactone, tetrachlorodecaoxide, tetrazomine, thaliblastine, thalidomide, thiamiprine, thiocoraline, thioguanine, thiotepa, thrombopoietin, thrombopoietin mimetic, thymalfasin, thymopoietin receptor agonist, thymotrinan, thyroid stimulating hormone, tiazofurin, tin ethyl etiopurpurin, tirapazamine, titanocene dichloride, topotecan hydrochloride, topsentin, toremifene, toremifene citrate, totipotent stem cell factor, translation inhibitors, trestolone acetate, tretinoin, triacetyluridine, triciribine, triciribine phosphate, trimetrexate, trimetrexate glucuronate, triptorelin, tropisetron, tubulozole hydrochloride, turosteride, tyrosine kinase inhibitors, tyrphostins, UBC inhibitors, ubenimex, uracil mustard, uredepa, urogenital sinus-derived growth inhibitory factor, urokinase receptor antagonists, vapreotide, variolin B, velaresol, veramine, verdins, verteporfin, vinblastine sulfate, vincristine sulfate, vindesine, vindesine sulfate, vinepidine sulfate, vinglycinate sulfate, vinleurosine sulfate, vinorelbine, vinorelbine tartrate, vinrosidine sulfate, vinxaltine, vinzolidine sulfate, vitaxin, vorozole, zanoterone, zeniplatin, zilascorb, zinostatin, zinostatin stimalamer, and zorubicin hydrochloride, as well as salts, homologs, analogs, polymorphs, derivatives, enantiomers, and/or functionally equivalent compositions thereof.

Other aspects of the invention are directed to kits. A “kit,” as used herein, typically defines a package or an assembly including one or more of the compositions of the invention, and/or other compositions associated with the invention, for example, as previously described. Each of the compositions of the kit may be provided in liquid form (e.g., in solution), or in solid form (e.g., a dried powder). In certain cases, some of the compositions may be constitutable or otherwise processable (e.g., to an active form), for example, by the addition of a suitable solvent or other species, which may or may not be provided with the kit. Examples of other compositions or components associated with the invention include, but are not limited to, solvents, surfactants, diluents, salts, buffers, emulsifiers, chelating agents, fillers, antioxidants, binding agents, bulking agents, preservatives, drying agents, antimicrobials, needles, syringes, packaging materials, tubes, bottles, flasks, beakers, dishes, frits, filters, rings, clamps, wraps, patches, containers, and the like, for example, for using, administering, modifying, assembling, storing, packaging, preparing, mixing, diluting, and/or preserving the compositions components for a particular use, for example, to a sample and/or a subject.

A kit of the invention may, in some cases, include instructions in any form that are provided in connection with the compositions of the invention in such a manner that one of ordinary skill in the art would recognize that the instructions are to be associated with the compositions of the invention. For instance, the instructions may include instructions for the use, modification, mixing, diluting, preserving, administering, assembly, storage, packaging, and/or preparation of the compositions and/or other compositions associated with the kit. In some cases, the instructions may also include instructions for the delivery and/or administration of the compositions, for example, for a particular use, e.g., to a sample and/or a subject. The instructions may be provided in any form recognizable by one of ordinary skill in the art as a suitable vehicle for containing such instructions, for example, written or published, verbal, audible (e.g., telephonic), digital, optical, visual (e.g., videotape, DVD, etc.) or electronic communications (including Internet or web-based communications), provided in any manner.

In some aspects, the present invention is directed to techniques for promoting one or more embodiments of the invention, e.g., as described above. As used herein, “promoted” includes all methods of doing business including, but not limited to, methods of selling, advertising, assigning, licensing, contracting, instructing, educating, researching, importing, exporting, negotiating, financing, loaning, trading, vending, reselling, distributing, repairing, replacing, insuring, suing, patenting, or the like that are associated with the systems, devices, apparatuses, articles, methods, compositions, kits, etc. of the invention as discussed herein. Methods of promotion can be performed by any party including, but not limited to, personal parties, businesses (public or private), partnerships, corporations, trusts, contractual or sub-contractual agencies, educational institutions such as colleges and universities, research institutions, hospitals or other clinical institutions, governmental agencies, etc. Promotional activities may include communications of any form (e.g., written, oral, and/or electronic communications, such as, but not limited to, e-mail, telephonic, Internet, Web-based, etc.) that are clearly associated with the invention.

In one set of embodiments, the method of promotion may involve one or more instructions. As used herein, “instructions” can define a component of instructional utility (e.g., directions, guides, warnings, labels, notes, FAQs or “frequently asked questions,” etc.), and typically involve written instructions on or associated with the invention and/or with the packaging of the invention. Instructions can also include instructional communications in any form (e.g., oral, electronic, audible, digital, optical, visual, etc.), provided in any manner such that a user will clearly recognize that the instructions are to be associated with the invention, e.g., as discussed herein.

U.S. Provisional Patent Application Ser. No. 61/156,676, filed Mar. 2, 2009, entitled “Methods and Systems for Treatment and/or Diagnosis,” by von Maltzahn, et al. is incorporated herein by reference.

The following examples are intended to illustrate certain embodiments of the present invention, but do not exemplify the full scope of the invention.

### Example 1

This example describes certain protocols and methods that may be useful in various embodiments of the invention.

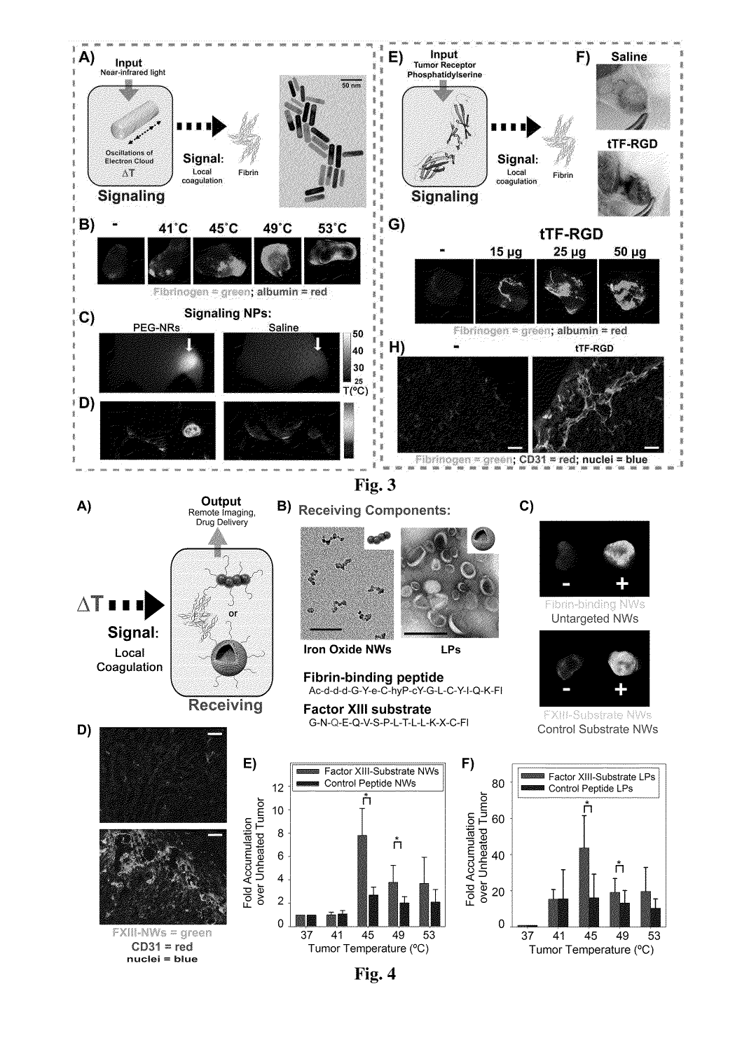

Poly(ethylene glycol)-coated gold nanorod (PEG-NR) synthesis (“signaling module”): PEG-NRs were synthesized as described in von Maltzahn, et al., “Computationally Guided Photothermal Tumor Therapy Using Long-Circulating Gold Nanorod Antennas, Cancer Res., 69(9):2009, incorporated herein by reference in its entirety. Briefly, highly stable, ˜13 nm×47 nm (FIG. 3A) CTAB (cetyltrimethylammonium bromide) coated gold nanorods with longitudinal plasmon resonance at 810 nm (Nanopartz, a division of Concurrent Analytical Inc., Salt Lake City, Utah) coated in 250 micrometer 5 kDa methyl-PEG-thiol (Laysan Bio, U.S., Arab, Ala.) were used. A solution of 5 kDa methyl-PEG-thiol and CTAB-coated gold nanorods was gently mixed at room temperature for 1 hour and dialyzed exhaustively against ultrapure water (18 megohm cm⁻¹) via cellulose ester membrane dialysis (Spectrapor, Rancho Dominguez, Calif.) to drive PEG addition. Dialyzed samples were filtered through 100 kDa filters (Millipore, Billerica, Mass.) to remove excess polymer, and stored at 4° C.

tTF-RGD and tTF-NGR Signaling component expression, purification, and in vitro testing: The cDNAs coding for the tTF-containing amino acids 1-218 and the respective C-terminal peptide extension were amplified by polymerase chain reaction (PCR) using the primers: 5′-CATGCCATGGGATCAGGCACTACAAATACTGTGGCAGCATATAAT-3′ (5′-primer) (SEQ ID NO: 21), 5′-CGGGATCCTATTATGGAGAATCACCTCTTCCTCTGAATTCCCC-3′ (3′-primer) (SEQ ID NO: 22) for tTF-RGD and 5′-CGGGATCCTATTATGCATGTGCTCTTCCGTTACCTCTGAATTCCCC-3′ (3′-primer) (SEQ ID NO: 23) for tTF-NGR. With the DNA-Ligation Kit (Novagen, Schwalbach am Taunus, Germany) the cDNA was cloned into the expression vector pET-30(+)a (Novagen) using the BamHI and NcoI sites of the vector. The vectors were introduced in competent Escherichia coli cells (BL21 DE3) according to the manufacturer's protocol (Novagen). The bacteria were cultivated in Luria broth medium supplemented with kanamycin (30 microgram/ml) at 37° C. When the bacteria cell suspensions reached an OD of ˜0.6, over-expression of the fusion proteins was initiated by adding 1 mM IPTG (Novagen). After ˜16 h, the cells were harvested and 5-7 ml lysis buffer (10 mM Tris-HCl, pH 7.5; 150 mM NaCl; 1 mM MgCl₂; 10 micrograms/ml aprotinin; 20 microliters benzonase; 2 mg/ml lysozyme) per gram wet weight were added. The lysed cells were incubated for 90 minutes at room temperature (RT) and centrifuged at 12,000 g for 20 min at 4° C. The pellet was resuspended and homogenized by sonicating in washing buffer (10 mM Tris/HCl, pH 7.5; 1 mM EDTA, 3% Triton X-100). To solubilize the inclusion bodies, 2-4 ml guanidinium buffer (6 M GuCl, 0.5 M NaCl, 20 mM Tris/HCl, pH 7.5; 1 mM DTT) per gram wet weight was added. After incubation overnight at room temperature (RT), the suspension was centrifuged at 10,000 g for 20 min at 4° C. and the supernatant was filtered through a 0.22 micrometer filter.

The solubilized tTF fusion proteins were refolded and purified by using a multi-step HPLC-based purification process (HPLC unit: AKTA purifier 100 System, GE healthcare, Uppsala, Sweden). It has an immobilized metal-(copper-)affinity chromatography (IMAC; IMAC Sepharose 6 FF, GE healthcare). The histidine-tagged tTF fusion proteins bind to the immobilized copper ions so that the complete refolding (gradient from 6 M to 0 M urea buffer within 60 min) and washing processes are performed on the column, from which the tTF proteins are eluted by applying 300 mM imidazole. During the subsequent gel filtration the IMAC eluate is conditioned by a buffer exchanging step (20 mM Tris/HCl, pH 8; 20% glycerol) using Sephadex G-25 (GE healthcare) in order to prepare for the following intermediate anion-exchange chromatography step (AIEX; Q Sepharose HP, GE healthcare; used buffers: 20 mM Tris/HCl, pH 8; 20% glycerol+/−300 mM NaCl). The concluding polishing step again comprises a gel filtration using Sephadex G25 in order to remove any remaining trace impurities and to exchange the buffer to PBS. The final protein solutions (>95% purity) are stored at −80° C. Each sample produced was tested for purity (SDS-PAGE, Western Blot, endotoxins, HPLC) and activity (Factor-X coagulation test).

Factor-X activation by tTF: The ability of the tTF proteins to enhance the specific proteolytic activation of Factor (F) X by FVIIa was assessed as follows. Briefly, to each well in a microtiter plate was added 20 microliters of: (a) 50 nM recombinant FVIIa (Novo-Nordisc, Bagsverd, Denmark) in TBS-BSA; (b) 0.16 nM-1.6 micromolar tTF/tTF-NGR in TBS containing 0.1% bovine serum albumine (BSA); (c) 25 nM CaCl₂ and 500 micromolar phospholipids (phosphatidylcholine/phosphatidylserine, 70/30, MM; Sigma, München, Germany). After 10 min at room temperature, 20 microliters of the substrate FX (Enzyme Research Laboratories, Swansea, UK) was added in a concentration of 5 micromolar. Aliquots were removed from the reaction mixture every minute and stopped in 100 nM EDTA. Spectrozyme FXa (American Diagnostica, Greenwich, Conn., USA) was added and rates of FXa generation were monitored by the development of color at 405 nm with a microplate reader (Bio-Rad, Hercules, Calif., USA).

Fibrinogen deposition in heated tumors: Bovine fibrinogen (Sigma) and albumin (Sigma) were reacted with near-infrared fluorochromes (VT750-NHS or VT680-NHS) at a 2:1 fluorophore:protein molar ratio in phosphate buffered saline pH 7.2 (PBS) for ˜2 hours and dialyzed extensively at 4° C. against PBS to remove unreacted fluorophores. The product of the dialysis was passed through a 0.1 micrometer filter, quantified using a BCA protein assay (Pierce), and assessed for fluorophore labeling via the absorption at the peak fluorphore absorbance (λₘₐₓ=750 and 680 nm, respectively, for VT750 and VT680). This reaction generated fibrinogen and albumin protein stocks carrying ˜1 fluorophore/protein. To assess whether fibrinogen and albumin home to heated tumors, ˜1 nanomole of both proteins (bearing distinct NIR-fluorophores) was injected intravenously into mice bearing bi-lateral MDA-MB-435 carcinoma tumors. Immediately following injection, one tumor on each mouse was externally heated using a temperature-controlled water bath set to between 41-53° C. for 20 min. At 24 hours post-injection, the mice were dissected and both tumors fluorescently imaged for the relative abundance of fibrinogen and albumin (LI-COR Odyssey Infrared Imaging System). To ensure that fluorophores did not optically or molecularly skew homing results, all fluorescent experiments were performed with equal numbers of mice allocated to VT750-fibrinogen/VT680-ablumin and VT680-fibrinogen/VT750-albumin administration at each temperature tested. Increases in protein tropism to heated tumors were analyzed by combining the fold increase in targeting observed for both fluorophore orientations (n=4 mice at each temperature tested).

Photothermal heating of passively-targeted PEG-NR in vivo: Nude mice were injected subcutaneously in the hind flank with ˜2×10⁶ MDA-MB-435 cells. After 2-3 weeks, the animals were anaesthetized with isoflurane and injected through the tail vein with PEG-NRs in 0.15 M NaCl, 0.1 M Na phosphate buffer, pH 7.2 (10 mg Au/kg, ˜150 microliter bolus). Biodistribution was assessed by collecting organ samples for inductively-coupled plasma mass spectrometry (Thermo-Scientific Finnigan ELEMENT2). The samples were frozen, lyophilized, and dissolved in aqua regia, prepared by adding 100 microliters of nitric acid and 300 microliters of 37% hydrochloric acid for 72 hours to dissolve gold particles. Then, samples were brought up into 10 ml of 9.6 ml 2% HNO₃ and analyzed via ICP-MS against standards. Control saline and organ samples with exogenously added PEG-NRs were utilized to calibrate the linearity of this method. All photothermal heating of NRs was conducted at 72 hours post administration (a time point after which they had completely cleared circulation) under the guidance of infrared thermography to continually illuminate the surface temperature of irradiated regions (FLIR Thermacam S60). A custom diode laser (RPMC Lasers Inc, 810 nm, 30 W) was utilized to broadly irradiate the right flank of tumor-bearing mice at ˜0.75 W/cm² and ˜1 W/cm² to maintain desired peak tumor temperatures in NR-injected mice (˜46° C. for initial fibrinogen-homing experiments and integrated signaling network implementation; 20 min exposure).

Immunohistochemical analysis in tumors: For histologic analysis, frozen sections of tumors were prepared. The sections were first fixed with acetone. Rat anti-mouse CD-31 (1:50, BD PharMingen) and biotinylated mouse fibrin(ogen) antiserum (1:50, Nordic) were used for immunochemical staining of tumor tissue sections. The corresponding secondary antibodies were added and incubated for 1 hour at room temperature: AlexaFluor-594 goat anti-rat or rabbit IgG (1:1,000; Molecular Probes), streptavidin Alexa Fluor 594 (1:1000; Molecular Probes). The slides were washed three times with PBS and mounted in Vectashield Mounting Medium with DAPI. At least three images from representative microscopic fields were analyzed for each tumor sample.

Peptide synthesis: Five peptides used in this example were: a fibrin-binding peptide (Ac-d-d-d-G-Y-e-C-hyP-cY-G-L-C-Y-I-Q-K-Fluorescein) (binding sequence in bold) (SEQ ID NO: 3), a peptide substrate for the transglutaminase Factor XIII (G-N-Q-E-Q-V-S-P-L-T-L-L-K-X-C-(K-Fluorescein)) (active glutamine in bold; X=6-aminohexanoic acid) (SEQ ID NO: 2), and a control substrate for FXIII (G-N-A-E-Q-V-S-P-L-T-L-L-K-X-C-(K Fluorescein)) (single amino acid substitution in bold) (SEQ ID NO: 12), the linear alpha-v-beta-3 targeting ligand matching the targeting motif on tTF-RGD (Fluorescein X C G R G D-S-P-COO-) (SEQ ID NO: 19), and the CD13/aminopeptidase N targeting ligand matching the motif on tTF-NGR (Fluorescein-X-C-G-N-G-R-A-H-A-COO-) (SEQ ID NO: 20). These were synthesized via standard FMOC solid-phase peptide synthesis (MIT Biopolymers Core or Tufts University Core Facility). In these sequences, Ac is the acetyl end terminus, d is D-aspartic acid, e is D-glutamic acid, hyP is hydroxproline, and cY is chlorotyrosine. The products were HPLC-purified to >90% purity and characterized via mass spectrometry. Additionally, high-affinity, cyclic alpha-v-beta-3-targeting peptides were purchased (Peptides International, Louisville, Ky.) for comparing the efficiency of communicating liposomes to liposomes targeted to endogenous tumor markers (cyclo(RGDfC). Fibrin-binding peptides were cyclized by bubbling air into 10 micromolar aqueous peptide solutions overnight, followed by lyophilization for subsequent use.

Nanoworm synthesis (“receiving module”): Superparamagnetic, dextran-caged iron oxide nanoworms (NWs) with a longitudinal size of ˜55 nm were synthesized, aminated using 20% v/v ammonium hydroxide, and derivatized with near-infrared fluorophores. Fibrin-binding peptides were attached to NWs via their exogenous lysine by first reacting fluorophore-labeled NWs with the bifunctional linker NHS-PEO5-NHS (Pierce) in phosphate buffered saline pH 7.2 (PBS) at a 5000:1 linker:NW molar ratio to prevent cross-linking. Following activation with linker, NWs were filtered using a gel filtration column (G50 media) and incubated overnight with cyclized fibrin-binding peptides at ˜1000:1 peptide:NW ratio with shaking. After ˜12 hours, NWs were purified from extra peptides by repeated filtration on centrifugal membrane filters (100 kDa size cutoff, Centricon, Millipore) and finally dispersed in PBS for spectrophotometric analysis of peptide labeling. Factor XIII-substrates, control substrates, alpha-v-beta-3 targeting ligands, and CD13/aminopeptidase N targeting ligands were attached to NWs via their exogenous cysteine similarly, but with the linker NHS-PEO12-maleimide (Pierce) in place of the bifunctional NHS-PEO5-NHS linker. All peptide-functionalized NWs were characterized via dynamic light scattering (DLS) and intravenously injected in vivo to ensure targeted NWs and control NWs exhibited similar circulation times. All peptide-modified particles carried ˜600 peptides/NW.

Doxorubicin-loaded liposome synthesis: Hydrogenated soy sn-glycero-3-phosphocholine (HSPC), cholesterol, and 1,2-distearoyl-snglycero-3-phosphoethanolamine-N-polyethylene glycol 2000 (DSPE-PEG(2k)), 1,2-distearoyl-sn-glycero-3-phosphoethanolamine-N-(maleimide(polyethylene glycol 2000)) (DSPE-PEG(2k)-MAL) were purchased from Avanti Polar Lipids (Alabaster, Ala.). Doxorubicin was purchased from Sigma Chemical Co. (St. Louis, Mo.). For peptide conjugation, liposomes with maleimide groups were prepared from HSPC, cholesterol, DSPE-PEG(2k), and DSPE-PEG(2k)-MAL in the molar ratio of 75:50:3:3 by lipid film hydration and membrane (100 nm) extrusion method. Encapsulation of doxorubicin (DOX) into the liposomes was then carried out using the pH gradient-driven loading protocol. Free doxorubicin was removed by gel filtration on Sephadex G-50. After doxorubicin loading, the maleimide-terminated liposomes were reacted with thiols on peptides (FXIII and control) for 2 hours and then purified by gel filtration on Sephadex G-50. The peptide-conjugated doxorubicin liposomes were stored in PBS at 4° C. before use.

In vitro cytotoxicity: Cytotoxicity assessments were conducted by the MIT Nanomaterials Toxicity Core using human HeLa cervical cancer cultures (ATTC) in 96-well plates grown to ˜70% confluency. Cells were incubated in quadruplicate with various dilutions of either PEG-NR or Lp formulations assessed for viability after 24 hours of incubation using the fluorogenic intracellular esterase sensor calcein acetoxymethylester (Invitrogen).

Quantifying NW homing to tumors: Mixtures of NIR-fluorophore-labeled, targeted and control NWs (bearing VT750 and VT680 or VT680 and VT750, respectively) were co-adminstered intravenously in PBS (2 mg Fe/kg) to tumor-bearing nu/nu mice to provide an internal control reference for coagulation-specific NW homing. At 24 hours post-NW injection, mice were sacrificed and organs were analyzed for both NIR-fluorophores (LI-COR Odyssey Infrared Imaging System). For integrated nanoparticle system characterization, mice were additionally imaged under isofluorane anaesthetic before euthanization using a whole animal fluorescence reflectance imaging system (Xenogen, IVIS Imaging System) to visualize the specificity of NW homing to tumors. Images from both organ scanning and whole animal imaging are displayed as overlaid fluorescent images from both channels (VT750 is green and VT680 is red). As with fibrinogen/albumin characterization earlier, all experiments were conducted with VT750-labeled targeted particles alongside VT680-labeled control particles and with VT680-labeled targeted particles alongside VT750-labeled control particles to ensure that fluorophore bias did not perturb results. For initial characterization, levels of targeted and control NW homing were plotted by comparing the average NW fluorescence in heated tumors versus unheated tumors (n=4 mice at each temperature). For integrated network evaluation, levels were plotted by comparing the average targeted and untargeted NW fluorescence in irradiated tumors versus un-irradiated tumors (n=4 mice for all conditions).

For autonomously-communicating nanosystems, NIR-fluorophore-labeled peptide-bearing NWs (bearing VT750 fluorophores) were intravenously (2 mg Fe/kg) in PBS to unanaesthetized MDA-MB-435 tumor-bearing nu/nu mice alone or alongside various tTF signaling components (25 micrograms). At 24 hours post-NW injection, the mice were sacrificed and organs were analyzed for NIR receiver fluorescence (LI-COR Odyssey Infrared Imaging System). For intraoperative fluorescent tumor imaging, mice were anaesthesized and tumours were surgically exposed to reveal detailed tumor fluorescence (LI-COR). The levels of NW homing to tumors were plotted by comparing the average NW fluorescence in fully-excised tumours (n=4 mice at each condition). For whole animal organ distribution, tTF-RGD signaling components were administered intraperitoneally (25 micrograms) and FXIII-NWs were administered intravenously (2 mg Fe/kg) to unanaesthetized mice bearing an MDA-MB-435 tumor.

Quantification of doxorubicin in tissues: MDA-MB-435 xenografts were established in nu/nu mice and administered with FXIII-substrate or control-substrate Lps as described in the manuscript. At 24 hours post-irradiation or external heating, doxorubicin in tissues was quantified fluorescently in organ homogenates. Briefly, organs were removed, weighed, incubated with 500 microliters of 70% EtOH, 0.3 N HCl, and homogenized (Tissue Tearor, Biospec Products) to release doxorubicin from tissues. Following homogenization, another 1 ml of 70% EtOH, 0.3 N HCl, was added to samples and they were centrifuged. Supernatants of samples were analyzed for doxorubicin fluorescence using a fluorescence microplate reader (Molecular Devices, SpectraMax GeminiEM) and compared to standard curves.

Therapeutic assessment of communicating and controlled nanoparticle signaling networks: Therapeutic studies were conducted by first intravenously administering PEG-NRs or saline into nu/nu mice bearing a single MDA-MB-435 tumor. At 72 hours post-injection, mice were intravenously administered FXIII-substrate Lps, control-substrate Lps, or saline (in a ˜150 microliter bolus) and broadly irradiated in the vicinity of the tumor with NIR light to supply the input to the inter-nanoparticle signaling network (810 nm, ˜1 W/cm², 20 min). An additional cohort of mice was administered saline at 0 and 72 hours and not exposed to NIR light in order to isolate any therapeutic efficacy of this input in isolation. Each therapeutic cohort included 7 mice, except for the unirradiated saline-only control, which had 6 mice. At regular intervals after treatment, tumors were measured and mice were weighed. Mice were sacrificed when tumors exceeded 500 mm.

### Example 2

This example illustrates synthetic components that were engineered to communicate with one another in vivo and amplify disease targeting. These systems were composed of signaling components (nanoparticles or proteins) that target tumors and then broadcast the tumor location to receiving nanoparticles in circulation that carry a diagnostic or therapeutic cargo, thereby amplifying their delivery. To facilitate robust communication between components, these systems were designed to harness ubiquitous biological cascades in plasma to transmit and amplify communications. It was shown that communicating nanoparticle systems can be composed from multiple types of signaling and receiving components, can transmit information via multiple molecular pathways, and can target over 40-fold higher doses of chemotherapeutics to tumors than non-communicating system components.

This example illustrates two-part nanosystems where signaling components target tumors and then broadcast the tumor's location to receiving nanoparticles in circulation (FIG. 2A). This figure is a schematic representation of communication between various components. In this figure, tumor-targeted signaling nanoparticles broadcast tumor location to receiving nanoparticles in circulation.

To facilitate rapid and efficient signal propagation between components, an endogenous biological cascade was harnessed (FIG. 2B). The coagulation cascade was selected due to its powerful amplification and positive feedback, ubiquitous presence in plasma, and potential to operate across multiple tumor types (FIG. 2C). Two signaling strategies were prepared to selectively activate the coagulation cascade in tumors: nanoparticles (gold nanorods) that target tumors and convert external electromagnetic energy into heat in order to locally disrupt tumor vessels; and engineered human proteins (tumor-targeted tissue factor) that autonomously survey host vessels for angiongenic tumor receptors and, in their presence, activate the extrinsic coagulation pathway (FIG. 2C). Receiving nanoparticles were constructed using nanomaterials with distinct functions: a model imaging agent (magnetofluorescent iron oxide nanoparticles) and a model therapeutic agent (doxorubicin-loaded liposomes). Also explored was the potential to communicate to receivers via two distinct molecular features of coagulation by developing peptide coatings that recognize fibrin directly and peptides that target coagulation enzyme activity by acting as a substrate for the coagulation transglutaminase Factor XIII (FXIII) (FIG. 2C).

Signaling and receiving components act as unnatural inputs and outputs to the coagulation cascade, respectively. The signaling components were either tumor-targeted plasmonic gold nanorods (NRs), which initiate coagulation cascade activation in tumors by photothermally disrupting tumor vessels and activating the extrinsic and intrinsic coagulation pathways, or tumor-targeted truncated tissue factor proteins, which are latent in circulation and activate the extrinsic coagulation pathway upon binding to tumor receptors. Communication was exploited to recruit inorganic (iron oxide nanoworms) or organic (drug-loaded liposomes) receiving components via activity of the coagulation transglutaminase FXIII or via targeting of polymerized fibrin.

The capacity of signaling components to precisely induce coagulation in tumors was first examined (FIG. 3A). To show that photothermal heating of gold nanorods (NRs) could compromise tumor blood vessels, the transduction of tumor heating into localized coagulation was examined by evaluating fibrin deposition in tumors as a function of temperature (FIG. 3A). This figure shows a schematic of nanorod-directed coagulation and transmission electron microscopy of near-infrared absorbing nanorods. Gold nanorods (NRs) were targeted to tumors to specify local coagulation cascade activation via photothermal conversion of near-infrared energy.

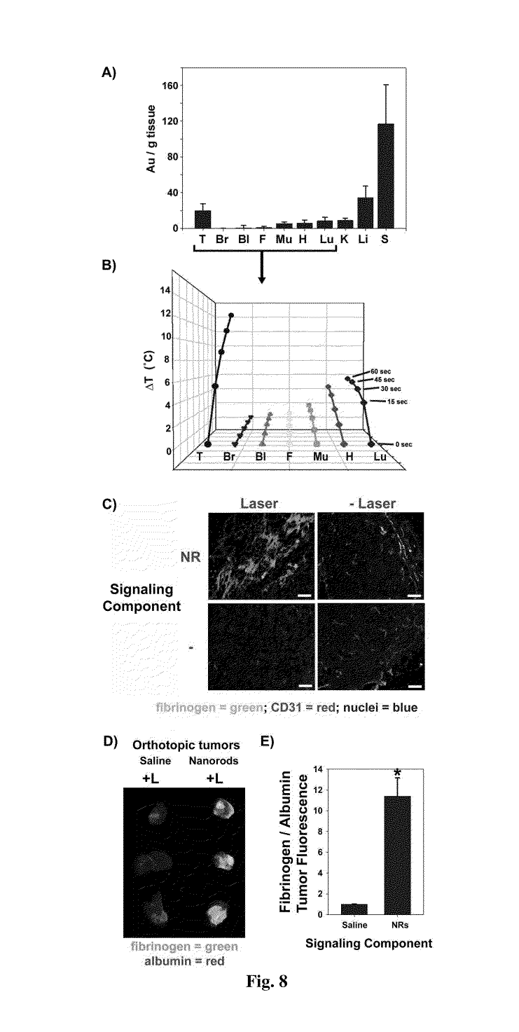

Purified fibrinogen (the precursor to fibrin) and albumin (an abundant blood protein unrelated to coagulation) were labeled with unique near-infrared fluorochromes to allow simultaneous assessment of coagulation-dependent and independent protein tropism to heated tumors. Mixtures of fibrinogen and albumin were intravenously injected into athymic (nu/nu) mice bearing bilateral human MDA-MB-435 tumors, after which one tumor was immediately heated using a temperature-controlled water bath. At 24 hours post-injection, the mice were sacrificed and dissected, and the relative levels of tumor fibrin(ogen) and albumin were assessed fluorescently. A marked induction of fibrin(ogen) accumulation in tumors was observed between 45° C. and 53° C., with little accompanying increase in albumin accumulation, indicating that heat specifically activated the coagulation cascade in tumors (FIGS. 3B and 7).

In addition, FIG. 7 shows that fibrinogen and albumin were labeled with unique near-infrared fluorochromes in the opposite channels as in FIG. 3A (VT680 and VT750, respectively), and injected in mice under identical conditions. Reversing the fluorophore labeling on fibrinogen and albumin enables quantification of protein tropism to heated tumors independent of any potential optical or molecular fluorophore bias. At 24 hours post-injection, mice were dissected and both tumors imaged for the relative abundance of fibrinogen and albumin.

Immunohistochemical staining for fibrin(ogen) in heated vs. unheated tumors from uninjected mice corroborated these findings, demonstrating that exogenous fibrinogen administration did not artificially drive accumulation in heated tumors (FIG. 7). Data taken under conditions of FIG. 3B and FIG. 7A were utilized to quantify the relative abundance of fibrinogen and albumin in heated tumors vs. unheated. At 45-53° C., fibrinogen abundance in tumors was significantly enhanced over albumin (P<0.05; 2-sided t-test; 4-mice per temperature). FIG. 7C shows the quantification of anti-fibrin(ogen) binding to sections from unheated and externally-heated tumors. As an independent measure of fibrin(ogen) deposition in heated tumors, uninjected mice bearing bilateral MDA-MB-435 tumors had one tumor immersed in a temperature-controlled water bath for 20 minutes and were sacrificed 24 hours later for histological sectioning. Fluorescent quantification showed significantly enhanced abundance of antibody binding at both 45° C. and 53° C. (P<0.01, 2-sided t-test, 6 separate regions analyzed in each condition).

Having probed the thermal sensitivity of coagulation in tumors, tumor-targeted gold NRs that could specify coagulation to occur in tumor tissues were then investigated. Rod-shaped gold nanoparticles are precisely-tunable plasmonic nanomaterials that may be synthesized in bulk, have narrow size distributions, and optical absorption coefficients 10⁴-10⁶-fold higher than conventional organic fluorochromes. Previously, it was demonstrated that polyethyleneglycol-coated gold NRs (PEG-NRs) have >17 hour circulation half-lives in mice and can passively target tumors in mice via their fenestrated angiogenic blood vessels to direct precise tumor heating with otherwise benign near-infrared (NIR) energy (FIG. 8). See, e.g., von Maltzahn, et al., “Computationally Guided Photothermal Tumor Therapy Using Long-Circulating Gold Nanorod Antennas, Cancer Res., 69(9):2009, incorporated herein by reference in its entirety.

Because near-infrared photons can penetrate several centimeters in human tissue, they provide an attractive external input to actuate vascular disruption in regionally-localized tumors. To examine NR-directed coagulation, PEG-NRs (10 mg Au/kg) or saline were intravenously administered to mice bearing bilateral MDA-MB-435 tumors (FIGS. 3C and 3D). FIG. 3C shows thermographic imaging of PEG-NR- and saline-injected mice under near-infrared irradiation of the right flank, while FIG. 3D shows fluorescence reflectance imaging of mice to visualize fibrinogen tropism to PEG-NR-heated tumors.

After PEG-NR clearance from circulation (72 hours post-injection), fluorescent fibrinogen was intravenously injected and the right flanks of mice were irradiated with near-infrared light (˜1 W/cm²), generating focal tumor surface temperatures of ˜49° C. in PEG-NR-injected mice, while saline-injected tumor surface temperatures remained below ˜37° C. (FIG. 3C). At 24 hours post-injection, irradiated tumors on NR-injected mice displayed localized accumulation of fibrinogen (FIG. 3D), while tumors with PEG-NRs or near-infrared energy alone and peripheral tissues lacked this feature. Histopathological analysis revealed that fibrin(ogen) deposition formed a broad interstitial mesh in heated tumors, indicating that NR heating could compromise tumor blood vessels to initiate extravascular coagulation (FIG. 8).

FIG. 8 shows nanorod-directed tumor heating and fibrinogen deposition in vivo. FIG. 8A shows PEG-nanorod biodistribution and targeting to MDA-MB-435 tumors 72 hours following intravenous administration, quantified via inductively-coupled plasma mass spectrometry (3 mice). (T=tumor, Br=brain, Bl=bladder, F=fat, Mu=muscle, H=heart, Lu=lung, K=kidney, Li=liver, S=spleen.) FIG. 8B shows ex vivo heating rates of dissected organs under identical irradiation conditions revealed that NR targeting confers specificity to photothermal tumor heating. Mice bearing a single MDA-MB-435 tumor were injected with PEG-NR signaling components and, 72 hours after injection, were dissected for comparison of photothermal organ heating rates.

FIG. 8C shows histopathological analysis of NR-directed fibrinogen deposition in tumors. Mice bearing bilateral MDA-MB-435 tumors were injected with PEG-NRs (10 mg Au/kg) or saline and, 72 hours later, injected with fluorescently-labeled fibrinogen (VT750) and broadly irradiated on their right side (˜1 W/cm², 810 nm, 20 min). At 24 hours post-injection, mice were sacrificed and tumors isolated for histological analysis of fibrinogen distribution (scale bars are 100 micrometers).

FIG. 8D shows photothermal induction of coagulation in orthotopic MDA-MB-435 tumors implanted deep in the mammary fat pad. Mice harboring a single MDA-MB-435 tumor in the mammary fat pad were injected with PEG-NRs (10 mg Au/kg) or saline and, 72 hours later, injected with fluorescently-labeled fibrinogen (VT750) and albumin (VT680) and broadly irradiated with extracorporeal light (˜1.5 W/cm², 810 nm, 20 min). At 24 hours post-injection, mice were sacrificed and tumors isolated for fluorescent imaging of relative fibrinogen and albumin accumulation. FIG. 8E shows quantification of fibrinogen:albumin fluorescence ratio in orthotopic MDA-MB-435 tumors. Data taken under conditions of FIG. 8D were utilized to quantify the relative abundance of fibrinogen and albumin in tumors from mice injected with saline and NRs and irradiated under identical conditions.

Next, the potential for a signaling component to autonomously survey the host vasculature for angiogenic tumor receptors was investigated, including, in their presence, engaging the extrinsic coagulation cascade. Such a system would operate without the need for any external electromangetic inputs (e.g. near-infrared energy) and amplify nanoparticle targeting to deep-seeded and disseminated cancers. A truncated, tumor-targeted version of the human protein tissue factor (tTF-RGD) was utilized, which harnesses an RGD peptide motif to induce coagulation upon binding to angiogenic alpha-v-beta-3 (αᵥβ₃)receptors (FIG. 3E). This figure shows a schematic of tumor-targeted tissue factor stimulation of the coagulation cascade in response to tumor receptors. The signaling components were ligand-targeted, truncated human tissue factor proteins (tTF-RGD) proteins that are latent in circulation and autonomously gain coagulation-inducing activity upon binding to alpha-v-beta-3 receptors in tumor blood vessels and associating with endothelial cell surface phosphatidylserine.

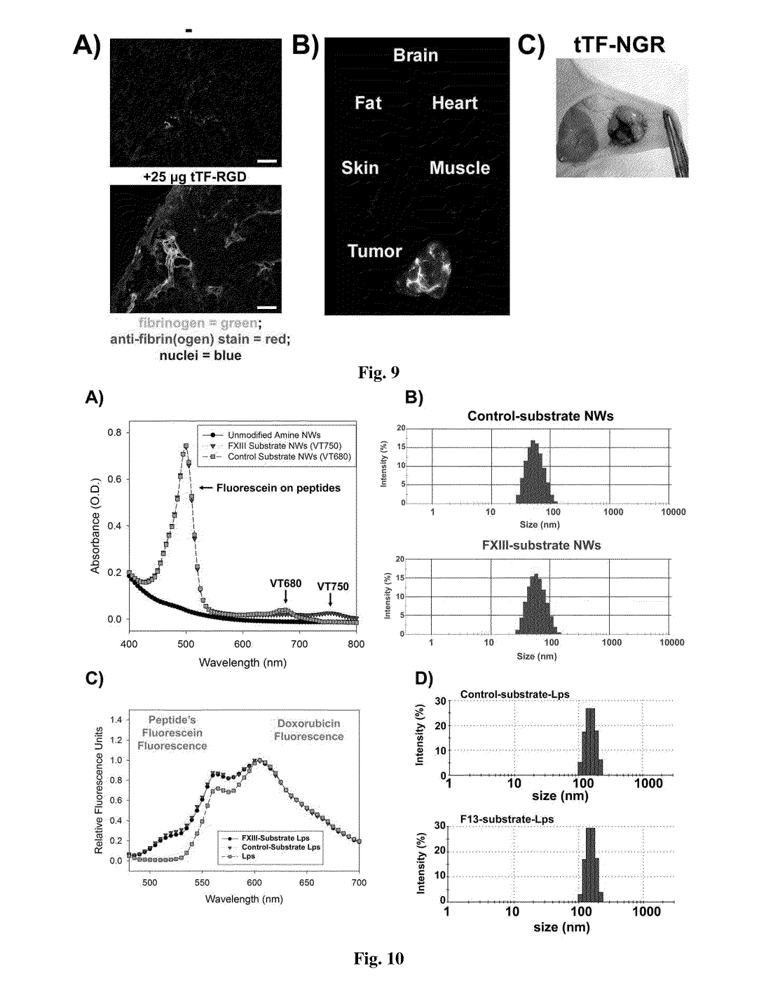

When tTF is separated from essential cell surface lipid co-factors, its activity alongside factor VII diminishes by 5 orders of magnitude. The differential activity of free and membrane-bound tTF has allowed tumor-targeted tTFs to safely and specifically activate the coagulation cascade by binding to endothelial receptors in mouse cancer models and human cancer patients. As with PEG-NR signaling components, the relative accumulation of fluorescently-labeled fibrinogen and albumin in MDA-MB-435 tumors of mice injected with varying doses of tTF-RGD proteins was first probed. At 24 hours after injection of doses greater than 15 micrograms tTF-RGD, a macroscopic appearance of hemorrhage in tumors that corresponded to the tumor-specific accumulation of fibrinogen in dendritic, vascular patterns was observed, which were absent in control tumors and other host organs (FIGS. 3F, 3G, and 9). FIG. 3F shows intraoperative images at 24 hours post-tissue factor injection revealing tTF-RGD-mediated hemorrhaging. Microscopically, this appearance of vascular coagulation was corroborated by the abundant localization of fibrin(ogen) within tumor blood vessels (FIG. 3G). In particular, FIG. 3G shows the coagulation-dependent and independent protein tropism to tumors on tTF-RGD-injected mice. tTF-RGD signaling components were injected intravenously at varying doses alongside mixtures of fluorescent fibrinogen (VT750) and albumin (VT680) to monitor tTF-RGD-mediated coagulation in tumors. FIG. 3H shows the histopathologic analysis of tumor fibrinogen distribution without (left) and with 25 micrograms tTF-RGD signaling component co-injection (right) (scale bars are 100 micrometers). It was found that both PEG-NR and tTF-RGD signaling components were capable of triggering tumor-specific coagulation, highlighting the potential for localized coagulation to communicate the location of the tumor to receivers in circulation.

FIG. 9 shows tTF-RGD and tTF-NGR Signaling component characterization. FIG. 9A shows an anti-fibrin(ogen) antibody stain that reveals little fibrin(ogen) in tumors of mice injected with saline+fluorescent fibrin(ogen) (top). Abundant fibrin(ogen) was observed in tumors of tTF-RGD-injected mice, which co-localized with injected fibrin(ogen) fluorescence (bottom), top=VT750-labeled fibrinogen without tTF-RGD injection; bottom=VT750-labeled fibrinogen with tTF-RGD injection (1 mg tTF-RGD/kg) (Scale bars=100 micrometers). FIG. 9B shows the macroscopic distribution of fluorescent fibrinogen in mice injected with tTF-RGD signaling component. Various organs were imaged at 24 hours following intravenous co-injection of fluorescent fibrinogen with tTF-RGD Signaling component (1 mg tTF-RGD/kg) (˜1 nmole fibrinogen; VT750 fluorophore). FIG. 9C shows intraoperative images at 24-hours post-tTF-NGR signaling component injection (which binds CD13/aminopeptidase N receptors associated with tumor angiogenesis) highlighting the modularity of tTF signaling components. Nu/nu mice bearing a single MDA-MB-435 tumor were intravenously injected with tTF-NGR (1 mg of tTF-NGR/kg) and dissected 24 hours later. Administration of both tTF-NGR or tTF-RGD led to the macroscopic appearance of tumoral hemorrhage and RBC stasis, while saline-injected mice displayed ivory, non-hemorrhagic tumors (see FIG. 3 for tTF-RGD and saline).

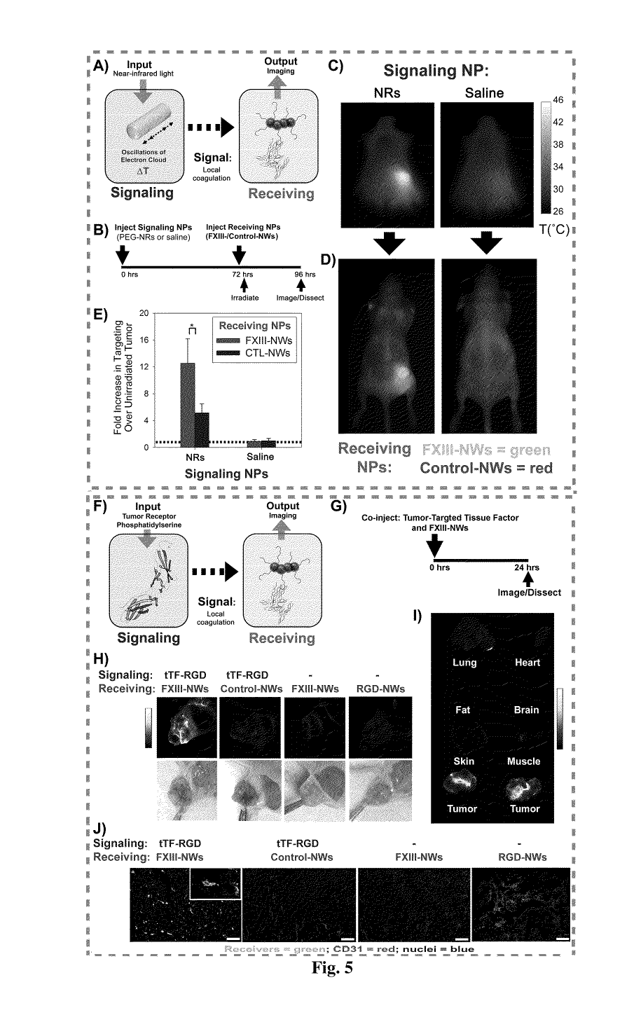

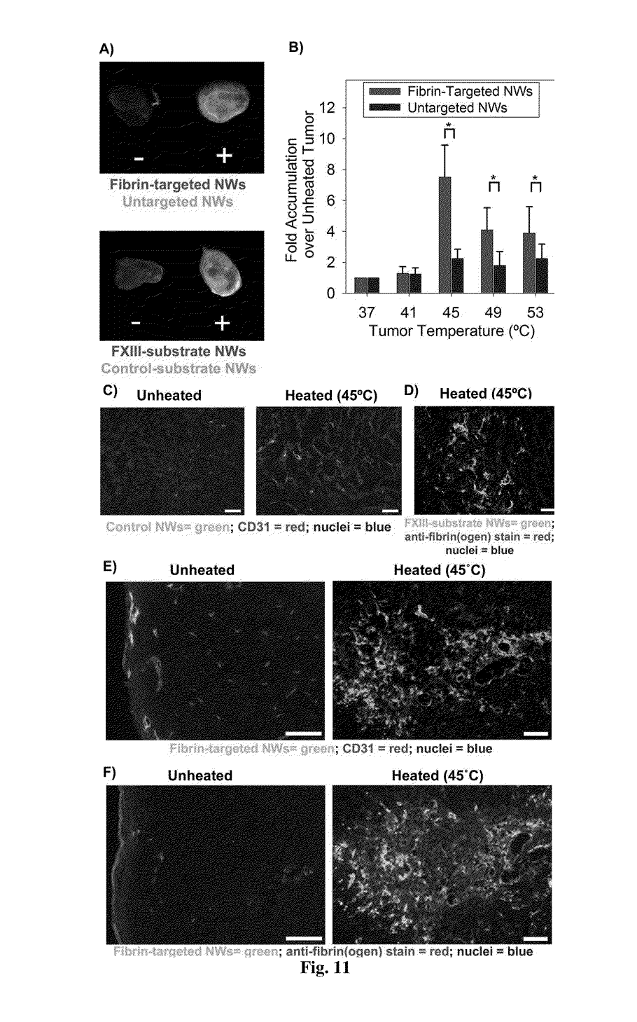

Next, receiving nanoparticles were developed that could efficiently target regions of coagulation to deliver therapeutics or act as imaging agents (FIG. 4A). This figure shows a schematic of receiving NP homing to regions of coagulation. Nanoworm (NW) imaging agents and drug-loaded liposomes (LPs) (top and bottom, respectively) were derivatized with coagulation-targeting peptides to form Receiving NPs. Initially, magnetofluorescent iron oxide nanoparticle imaging agents (NWs) (FIG. 4B, top) were derivatized with a peptide substrate for the coagulation transglutaminase Factor XIII (G-N-Q-E-Q-V-S-P-L-T-L-L-K-X-C-Fluorescein) (SEQ ID NO: 2, X is a 6-aminohexanoic acid) to create receivers that would covalently incorporate in regions of active coagulation (FIG. 4B, bottom; FIG. 10).

FIG. 4B shows transmission electron microscopy images of the two classes of nanomaterials utilized in receiving NP synthesis: iron oxide nanoworms (NWs; scale bar is 50 nm) and doxorubicin-loaded liposomes (LPs; scale bar is 400 nm). Two peptides were utilized to generate receiving NPs: a fibrin-binding peptide and a glutamine-containing substrate for the coagulation transglutaminase FXIII to respectively direct particle binding and covalent attachment in regions of coagulation.

FIG. 10A shows spectrophotometric characterization of NW receiving component functionalization. Aminated NWs were conjugated with NHS-activated NIR fluorochromes (VT680 or VT750) to allow fluorescent imaging and subsequently linked to thiol-containing FXIII-substrate peptides or control-peptides. The spectra NWs were utilized to quantify the number of peptides and NIR-fluorochromes per particle (˜600 FXIII- or control-peptides/NW and ˜12-15 fluorochromes/NW, respectively). Conjugation conditions were optimized to produce populations with approximately equal numbers of peptides in the FXIII-NWs and control-NWs. FIG. 10B shows dynamic light scattering (DLS) characterization of FXIII-NW and control-NW receiving components. After peptide functionalization with NIR-fluorochromes and peptides, samples were analyzed via DLS to probe the hydrodynamic size of each conjugate. FIG. 10C shows fluorescent characterization of FXIII-LPs and control-LPs. The fluorescence emission spectra of Fluorescein-containing FXIII- and control-peptides was utilized to ensure similar surface density on LP conjugates (excitation: 444 nm; cutoff: 455 nm; emission 480 nm-700 nm). FIG. 10D shows dynamic light scattering characterization of FXIII-LP and control-LP receiving components. After peptide functionalization, LP receiving components were analyzed via DLS to probe the hydrodynamic size of each conjugate.

Having observed that external heating of tumors produced localized coagulation, this response was utilized in an assay to assess the ability of receivers to target tumor coagulation prior to integrating them with signaling components. Mixtures of targeted and untargeted NWs, labeled with unique near-infrared fluorochromes, were intravenously injected into mice bearing two MDA-MB-435 tumors. Immediately following injection, one tumor was submerged in a temperature-controlled water bath for 20 min and the mice were dissected at 24 hours for fluorescent organ imaging.

It was found that the accumulation of FXIII-substrate receiving NWs was sharply amplified at 45° C. compared to control-NWs bearing peptides without the essential glutamine for FXIII cross-linking (FIGS. 4C, 4D, and 11), enabling nearly an order of magnitude increase in tumor targeting compared to unheated tumors.

FIG. 4C shows fluorescence reflectance imaging of receiving NP homing to externally-heated tumors. Mixtures of targeted and untargeted NWs, labeled with the unique NIR-fluorochromes VT750 and VT680, respectively, were intravenously injected into mice bearing bilateral MDA-MB-435 tumors. Immediately following injection, one tumor was submerged in a temperature-controlled water bath for 20 min and mice were dissected at 24 hours for fluorescent organ imaging. Overlaid fluorescence images are shown for targeted and untargeted receiving NP accumulation in both heated (+, 45° C. heating) and naive (−) tumors from the same mouse. FIG. 4D shows histopathological analysis of receiving NP homing to heated tumors. Histological sections from naive (top) and heated (bottom, 45° C.) tumors in FXIII-NW-injected mice were stained for CD31 and nuclei and imaged to reveal receiving NP distribution. (Scale bars are 100 micrometers.)

The specificity of heat-induced targeting to coagulation persisted at higher temperatures, although the magnitude of accumulation decreased (FIG. 4E), likely indicating that higher temperatures accelerated intravascular coagulation and occlusion, diminishing the perfusion required for delivery of receiving NWs into tumors. Histopathologically, FXIII-substrate NWs showed marked extravasation and interstitial spreading in heated tumors compared with controls (FIGS. 4D and 11), illustrating the capacity of thermal energy to dismantle tumor vascular barriers and direct abundant interstitial receiver accumulation.

In particular, FIG. 4E shows a quantification of the amplification of FXIII-substrate and control NW receiver homing to heated over unheated tumors. The fold enhancement of NW targeting is plotted across the range of temperatures tested (p=0.02 and 0.03 for the difference between FXIII-substrate-NWs and control substrate NWs at 45° C. and 49° C., respectively; paired, two-sided t-test, n=4; error bars are standard deviations).

FIG. 11A shows fluorescence reflectance imaging of receiving component homing to externally-heated tumors. Mixtures of targeted and untargeted NWs (labeled with the opposite orientation of NIR-fluorochromes used in FIG. 4C to control against potential optical or molecular fluorochrome bias to receiving component detection in heated vs. unheated tumors) bearing VT680 and VT750 fluorochromes, respectively, were intravenously injected into mice bearing bilateral MDA-MB-435 tumors. Immediately following injection, one tumor was submerged in a temperature-controlled water bath for 20 min and the mice were dissected at 24 hours for fluorescent organ imaging. Overlaid fluorescence images are shown for targeted (red) and untargeted (green) Receiving component accumulation in both heated (“+”, 45° C. heating) and naive (“−”) tumors from the same mouse.

FIG. 11B shows fluorescent quantification of fibrin-binding and untargeted-NW receiving component homing to heated over unheated tumors. The fold enhancement of NW targeting is plotted across the range of temperatures tested (n=4 mice in each set, p<0.05 for the difference between fibrin-binding-NWs and untargeted-NWs at 45° C., 49° C., and 53° C., respectively, 2-tailed t-test). FIG. 11C shows histopathological analysis of control-substrate NWs. Mice bearing bilateral MDA-MB-435 tumors were injected with control-substrate NWs and one tumor was heated to 45° C. for 20 min. At 24 hours post-injection, mice were sacrificed and tumors were analyzed for NW distribution in histology. (Scale Bar is 100 Micrometers.)

FIG. 11D shows co-localization of FXIII-substrate NWs with areas of anti-fibrin(ogen) staining. Mice bearing bilateral MDA-MB-435 tumors were injected with FXIII-substrate NWs and one tumor was heated to 45° C. for 20 min. At 24 hours post-injection, the mice were sacrificed and tumors were analyzed for NW distribution in histology using the same exposure conditions for NW imaging as FIGS. 14, 15, and 4D. (Scale bar is 100 micrometers.) FIG. 11E shows the histopathological distribution of fibrin-targeted NWs in unheated and heated tumors (Scale bar is 100 micrometers.) FIG. 11F shows co-localization of fibrin-targeted NWs with anti-fibrin(ogen) antibody staining in heated tumors. (Scale bar is 100 micrometers.)

Also explored was feasibility of communicating via an alternative molecular pathway in the coagulation cascade. NWs were derivatized with a fibrin-binding peptide (Ac-d-d-d-G-Y-e-C-hyP-cY-G-L-C-Y-I-Q-K-Fluorescein) (FIG. 4B) (SEQ ID NO: 3) and tested in a similar assay. Fibrin-binding receiving NWs also exhibited nearly a 10-fold amplification of targeting to heated tumors (FIGS. 4C and 11), with prominent extravascular accumulation histopathologically (FIG. 11).

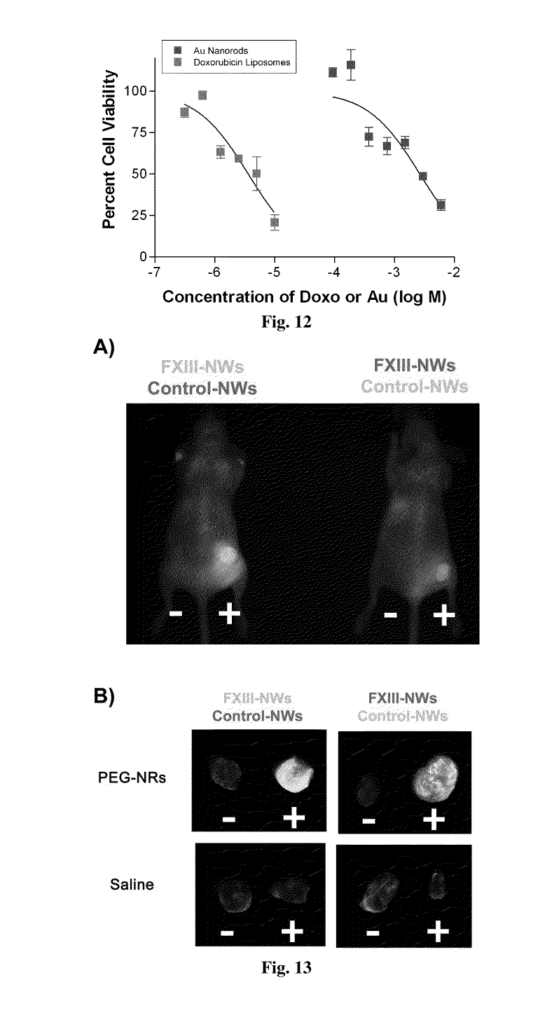

Next, model therapeutic receiving nanoparticles were constructed to provide amplified drug delivery to regions of tumor coagulation. Therapeutic receivers were developed by synthesizing doxorubicin-loaded liposomes (LPs) with tethered FXIII substrates (FIGS. 10 and 12). Here, tumor heating to 45° C. directed the accumulation of over 40-times higher doses of doxorubicin in tumors compared with unheated controls and significantly enhanced targeting over control LPs (FIG. 4F). This figure shows the quantification of the amplification of FXIII-substrate and control drug-loaded liposome receiver homing to heated over unheated tumors. The fold enhancement of doxorubicin accumulation in tumors is plotted across the range of temperatures tested for FXIII-substrate LPs and control-substrate LPs (p=0.025 and p=0.049 for the difference between FXIII-substrate-NWs and control substrate NWs at 45° C. and 49° C., respectively; unpaired, two-sided t-test, n=3; error bars are standard deviations).

FIG. 12 shows cytotoxicity experiments to assess intrinsic toxicity of Au nanorods and doxorobicin-loaded liposomes. Cytotoxicity assessments were conducted using Human HeLa cervical cancer cultures (ATTC) in 96-well plates grown to ˜70% confluency. The cells were incubated with various dilutions of either PEG-NR or LP formulations assessed for viability after 24 hours of incubation using the fluorogenic intracellular esterase sensor calcein acetoxymethylester (each point represents the average of 4 wells in a 96-well plate).

### Example 3

Having developed signaling and receiving components and characterized their function in isolation as discussed above, this example illustrates the ability of integrated nanoparticle systems to communicate and amplify tumor targeting in vivo (FIG. 5A). First, the ability for communication between PEG-NR signaling and FXIII-NW receiving components to amplify tumor imaging was tested.

PEG-NRs (or saline) were intravenously-injected into mice bearing bilateral MDA-MB-435 tumors. After NR clearance from circulation (72 hours), mixtures of FXIII-substrate and control-NWs labeled with distinct near-infrared fluorochromes were co-injected intravenously, followed by near-infrared irradiation of the entire right flank of the mouse (˜0.75 W/cm², 810 nm, 20 min) under infrared thermographic observation. At 96 hours, the entire mouse and then the individual explanted organs were fluorescently imaged (FIG. 5B, which shows the experimental timeline for testing communicating nanoparticles).

Thermographic surveillance of photothermal heating showed focal tumor heating only in NR-injected mice (FIG. 5C) and whole-animal imaging at 96 hours revealed pronounced homing of FXIII-targeted NWs to NR-heated tumors, with over an order of magnitude increase in accumulation above unirradiated contralateral tumors and tumors on saline-injected mice (FIGS. 5D, 5E, and 13). At 72 hrs post NR- or saline-injection (10 mg Au/kg), mice were co-injected with targeted (FXIII-NWs) and untargeted (Control-NWs) and their right flanks were broadly irradiated (810 nm, ˜0.75 W/cm², 20 min) under infrared thermographic surveillance to reveal surface temperatures. FIG. 5D illustrates overlaid fluorescence reflectance image of targeted and untargeted receiving NP homing. At 24 hours post-irradiation, whole-animal fluorescence imaging revealed the distributions of targeted (FXIII-NWs) and untargeted (control-NWs) receiving NPs. FIG. 5E shows quantification of receiving NP homing in irradiated vs contralateral unirradiated tumors. After whole-animal imaging, mice were dissected and the fluorescence of each tumor was measured to quantify the homing of receiving NPs. (* indicates p=0.02, paired, two-sided t-test; n=4; error bars are standard deviations).

FIG. 13 shows integrated communicating nanoparticles from FIG. 5D, but with inverted fluorophore-targeting ligand relationships to control against bias and ex-vivo imaging of excised tumors. FIG. 13A shows simultaneous near-infrared imaging of co-injected: VT750-labeled, FXIII-substrate-NWs (left mouse) and VT680-labeled, control-substrate NWs (left mouse); or VT680-labeled, FXIII-NW (right mouse) and VT750-labeled, control-substrate NWs (right mouse). The inversion of fluorochrome-NW relationships prevents optical or molecular bias from fluorophores in homing visualization. +indicates broad laser irradiation (810 nm, 0.75 W/cm², 20 min). FIG. 13B shows imaging of NW homing in excised tumors from experiments in FIGS. 5D and 5E. Each box was imaged using the same acquisition parameters for both VT750 and VT680 and contains the left and right tumors from MDA-MB-435-bearing mice.

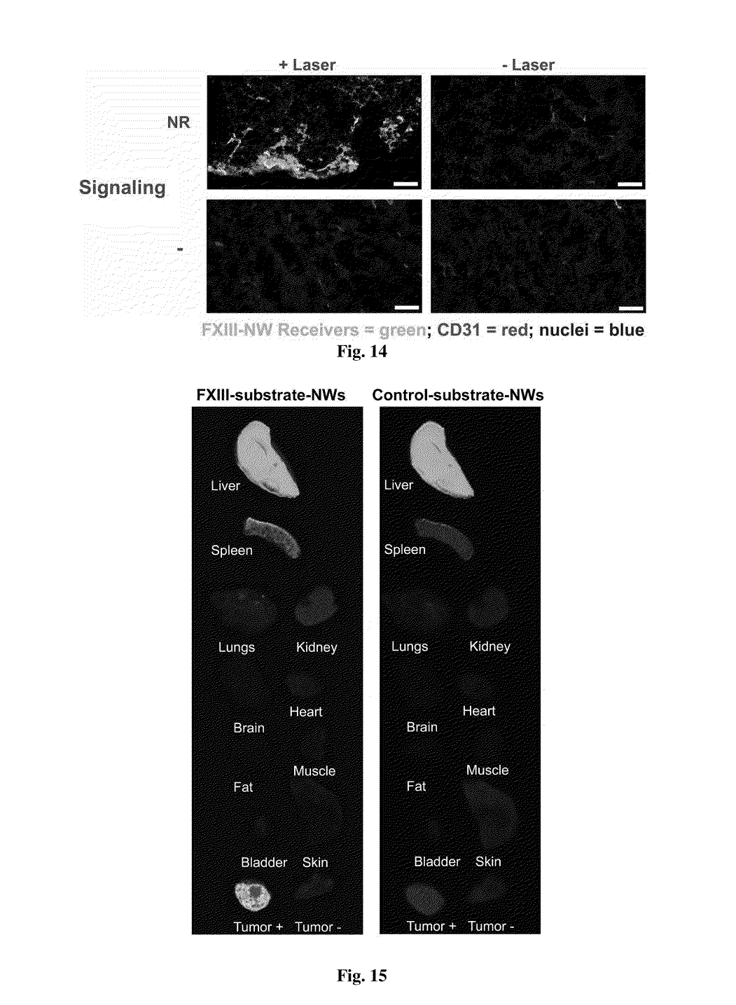

istologically, integrated nanoparticle systems produced intense regions of FXIII-NW fluorescence relative to controls, particularly in tumor boundaries where blood vessels were well-perfused (FIG. 14). Communicating nanoparticle systems were found to be effective in xenograft cervical tumor models as well, directing several fold amplification in homing of targeted receiving nanoparticles over untargeted controls (FIG. 15).

FIG. 14 shows histopathological analysis of FXIII-targeted NWs with integrated and disconnected NP communication systems. These are the histopathological sections from the integrated NP signaling experiments in FIGS. 5C-5E and 13. At 24 hours post-NW injection, mice were sacrificed and tumors were analyzed for FXIII-NW distribution in histology. (Scale bars are 100 micrometers.)

FIG. 15 shows the function of NP communication in human cervical xenograft tumors. Athymic (nu/nu) mice bearing bilateral Hela human cervical cancer tumors were injected with PEG-NRs and, 72 hours later, a mixture of FXIII-substrate-NWs and control-substrate-NWs, labeled with unique NIR-fluorophores, as described for MDA-MB-435 tumor-bearing mice in FIG. 5. Immediately following injection of NW mixtures, the right flanks of mice were broadly irradiated with a diode laser source. At 24 hours post-injection, the homing of FXIII-NWs and control-NWs was visualized using NIR fluorescent organ imaging. (“Tumor +” indicates the irradiated tumor; “Tumor −” was not exposed to diode irradiation.)

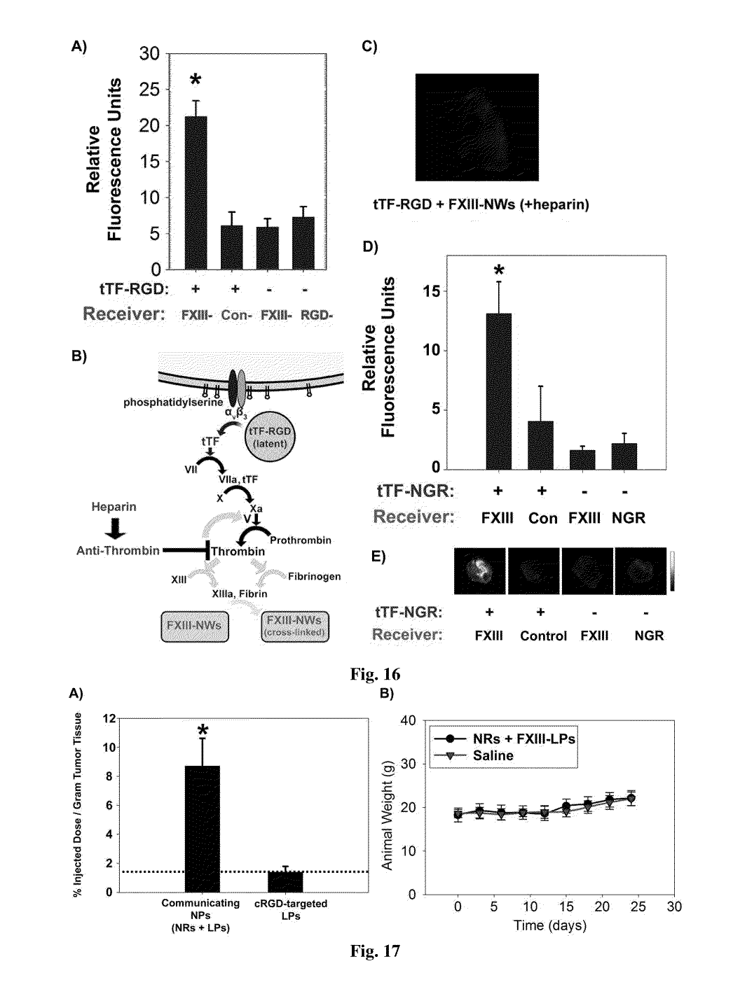

Next, the ability for autonomous communication between tTF-RGD signaling and FXIII-NW Receiving components was probed to amplify tumor targeting without the need for an external energy source (FIG. 5F). FIG. 5F shows a schematic of a nanosystem that communicates autonomously in the presence of tumor receptors. When co-injected alongside FXIII-NW Receivers (FIG. 5G), it was found that tTF-RGD signaling components amplified receiver targeting by several fold over non-communicating controls and over NWs that were directly targeted by the linear RGD targeting motifs that direct tTF homing (FIGS. 5H and 16). FIG. 5G shows the experimental timeline for testing the autonomous nanosystem in vivo.

FIG. 5H shows intraoperative imaging of NW receivers. Nu/nu mice bearing a single MDA-MB-435 tumor were intravenously injected with communicating (tTF-RGD+FXIII-NWs) or control (tTF-RGD+control-NWs) systems, FXIII-NWs alone, or NWs targeted by the peptide used to direct signaling component tumor homing (1 mg/kg tTF-RGD). At 24 hours post-injection, tumors were surgically exposed for fluorescent intraoperative imaging of NW homing. Similar to the fibrin(ogen) distribution observed during tTF-RGD Signaling component testing, FXIII-targeted NW Receivers injected alongside tTF-RGD proteins produced a dendritic pattern of accumulation in tumors, corresponding to abundant intravascular localization immunohistochemically (FIGS. 5H, 5I, and 5J). This amplified vascular targeting was found to be highly specific for tumors over normal organs and was absent when the coagulation inhibitor heparin was administered alongside the integrated system (FIGS. 5I and 16). Further, it was found that tTF-NGR Signaling components, which target CD13 angiogenic receptors, were also able to amplify receiver targeting to tumors above controls (FIG. 16), highlighting the capacity for autonomously-communicating systems to be customized for specific molecular signatures of disease.

FIG. 5I shows tumor specificity of the autonomous nanosystem. Excised organs from mice injected with autonomously communicating nanosystems (tTF-RGD+FXIII-NWs) were imaged for NW fluorescence at 24 hours post-injection (1 mg/kg tTF-RGD). FIG. 5J shows histopathological analysis of NW receivers. Histopathological sections from experiments in FIG. 5H. At 24 hours post-NW injection, mice were sacrificed and tumors were analyzed for NW receiver distribution in histology. (RGD-NW scale bar is 100 micrometers; all others are 200 micrometers).

FIG. 16 shows the characterization of autonomous communication between tTF-RGD signaling components and FXIII-NW receivers. FIG. 16A shows fluorescent quantitation of NW homing autonomously-communicating nanosystems and controls from mice in experiment from FIG. 5H (Con=Control) (*p<0.05 versus all other sets, n=4 mice for each set). FIG. 16B shows a schematic of heparin-mediated disruption of nanosystem communication. FIG. 16C shows nu/nu mice bearing a single MDA-MB-435 tumor were intravenously injected with the autonomous nanosystem (tTF-RGD+FXIII-NWs; 1 mg/kg tTF-RGD) alongside heparin to prohibit thrombin activation (intravenous heparin bolus of 800 units/kg+intraperitoneal bolus of 500 units/kg 30 minutes later). At 24 hours post-injection, mice were sacrificed and tumors were surgically removed for fluorescent imaging of NW localization to tumors. Tumors on heparin-injected mice lack characteristic vascular pattern of communication between tTF-RGD signaling components and FXIII-NW receivers. FIG. 16D modularity of autonomously-communicating nanosystems. tTF-NGR Signaling components (which bind CD13/aminopeptidase N receptors associated with tumor angiogenesis) were substituted for tTF-RGD signaling components to explore whether tTF signaling modules with varying receptor specificities could be coupled into communicating nanosystems. Nu/nu mice bearing a single MDA-MB-435 tumor were intravenously injected with communicating (tTF-NGR+FXIII-NWs) or control (tTF-NGR+control-NWs) nanosystems, FXIII-NWs alone, or NWs targeted by the peptide used to direct tTF-NGR signaling component tumor homing (1 mg/kg tTF-NGR). At 24 hours post-injection, mice were sacrificed and tumors were surgically removed for fluorescent imaging of NW localization to tumors. FIG. 16E shows the quantification of average fluorescence from tumor cohorts above (n=3 or 4 mice in each set; error bar indicates standard deviation of data; Student's two-tailed t-test: p<0.02 for tTF-NGR+FXIII-NWs vs. all other treatment sets).

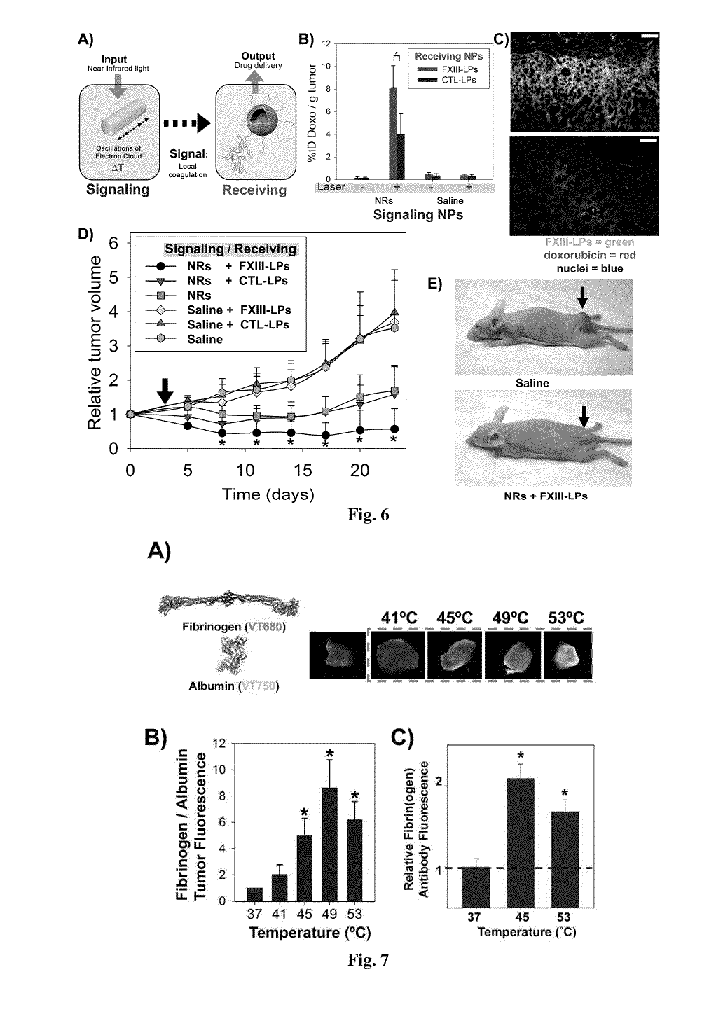

As a proof-of-principle that nanoparticle communication could improve tumor therapy, the ability of a communicating nanosystem to improve drug delivery and delay tumor progression was next studied (FIG. 6A, which shows a schematic of a therapeutic system of communicating nanoparticles). It was found that communication between NR signaling components and FXIII-LP receivers amplified the accumulation of doxorubicin in tumors by over 40-fold (˜8% ID/g) as compared to the LPs alone (FIG. 6B). This figure shows the quantification of doxorubicin-loaded LP receiver homing in irradiated vs. contralateral unirradiated tumors. At 96 hours after signaling NP injection, mice were dissected and the doxorubicin fluorescence of each tumor homogenate in acidic ethanol was measured to quantify the homing of Receiving NPs. (* indicates p=0.021, unpaired, two-sided t-test, n=4; error bars are standard deviations).

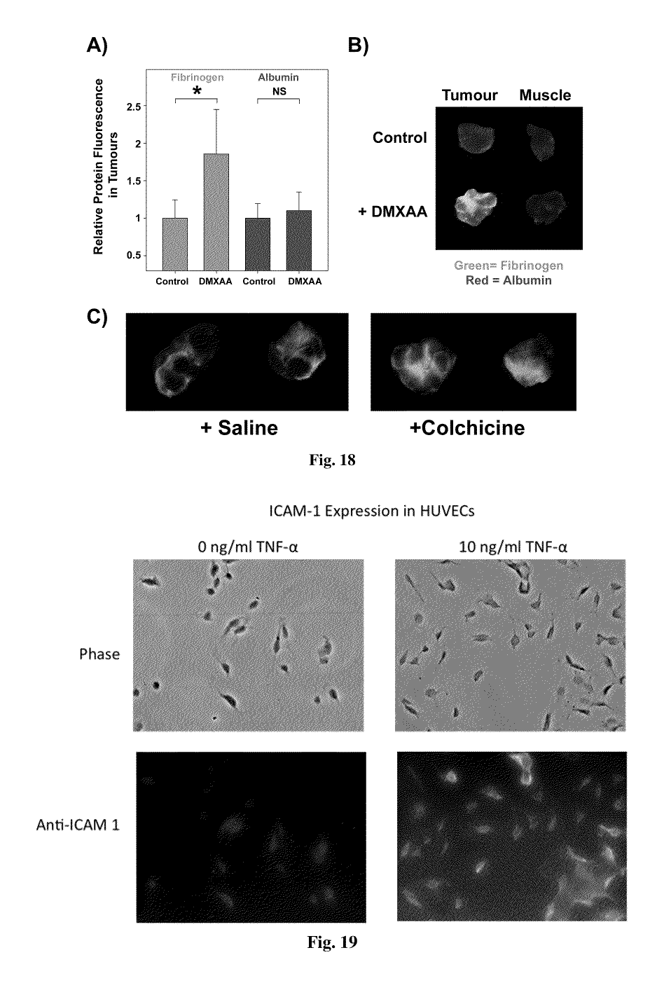

When compared to an optimized liposome formulation that targeted endogenous vascular receptors (alpha-v-beta-3 for high affinity cyclic-RGD peptide-targeted LPs), the integrated nanosystem deposited more than 6-fold more drug in tumors, illustrating the potential for nanoparticle communication to amplify drug delivery over nanoparticles targeted directly to tumor receptors (FIG. 17). FIG. 17A shows a comparison between communicating LPs and an optimized formulation of LPs designed to target the endogenous tumor markers alpha-v-beta-3 via high-affinity cyclic RGD peptides. Communicating NPs deliver over 6-fold higher doses of doxorubicin per gram of tumor tissue. FIG. 17B shows animal weights for mice treated with therapeutic nanoparticle systems compared with those administered saline only.

This amplification of drug delivery likely has at least two components: heat-dependent increases in passive accumulation due to improved extravasation in tumors (as indicated by control-LPs and consistent with previous observations) and specific biochemical recognition of the amplified coagulation cascade products by the peptide coating. Histologically, FXIII-LPs formed a broad interstitial mesh in NR-heated tumors, with released doxorubicin fluorescence emanating from the nuclei of surrounded tumor cells, confirming the delivery and release of active drug within tumor tissues (FIG. 6C). At 24 hours post-NW injection, the mice were sacrificed and tumors were analyzed for FXIII-LP and doxorubicin distributions in histology. (Scale bars are 100 micrometers). Finally, the therapeutic efficacy of these communicating nanoparticles in mice bearing single MDA-MB-435 human carcinoma tumors was evaluated. PEG-NRs (10 mg/kg) or saline were injected into mice and, once NRs had cleared from circulation (72 hrs), a single intravenous dose (2 mg/kg doxorubicin) of FXIII-substrate-LPs, control-substrate-LPs, or saline was given, followed immediately by irradiation with near-infrared energy (˜0.75 W/cm², 810 nm, 20 min). It was found that a single treatment with communicating nanoparticles directed a prolonged inhibition of tumor growth that was significantly more effective than system components in isolation (FXIII-LPs, control-LPs, NRs) and non-communicating control systems (NRs+control-LPs) (FIGS. 6D and 6E) (P<0.05 for NR+FXIII-LPs compared to all other treatment groups at each day from 8-24 after treatment; ANOVA) without detectable weight loss due to systemic toxicity (FIG. 17).

FIG. 6D shows tumor volumes following a single treatment with communicating nanoparticle systems and controls. Tumors in all treatment groups except saline (-laser) were exposed to near-infrared irradiation for 20 min (˜0.75 W/cm², ˜810 nm, arrow) 72 hrs after i.v. nanorod or saline injection (P<0.05 for NR+FXIII-LPs and all other treatment sets between days 8 and 24; ANOVA, n=7 mice in each set). FIG. 6E shows representative images of mice treated with communicating nanoparticles (NRs+FXIII-LPs, below) compared with untreated controls (Saline, above) (20 days post-treatment).

Inspired by communication in biological systems, these nanoparticles communicate to amplify disease targeting. In contrast with existing “combination” therapies that act synergistically but distribute independently in vivo, the strategy shown in these examples is composed of agents that communicate to more efficiently find and treat diseases. It was demonstrated that systems of synthetic human proteins and simple nanoparticles can be engineered to communication the location of tumors by harnessing the coagulation cascade. In addition to the two signaling components explored here, a number of cancer therapies in clinical practice or trials have been shown to induce local coagulation in tumors, including vascular disrupting agents (i.e. tubulin binding-drugs:; small molecule cytokine inducers; ligand-targeted cytokines; and ligand-targeted pro-coagulants), photodynamic therapies, anti-vascular antibodies, hyperthermia/ablation, among others.

To investigate whether small molecules could act as signaling components, the induction of coagulation in tumors following treatment with either the cytokine-inducing drug DMXAA (5,6-dimethylxanthenone-4-acetic acid) or the tubulin-binding drug colchicine was probed. It was found that both agents specifically induced coagulation in tumors (FIG. 18), showing that small molecule signaling platforms may also be harnessed. FIG. 18A shows quantifying fibrinogen and albumin homing to tumors in mice injected with the cytokine-inducing vascular disrupting agent DMXAA. Mice bearing MDA-MB-435 tumors were intravenously injected with a mixture of fibrinogen and albumin that had been labeled with unique near-infrared fluorophores (VT750 and VT680, respectively). Immediately after injection, mice were intraperitoneally injected with DMXAA (20 mg/kg) in 200 microliters of sodium bicarbonate buffer or vehicle. At 24 hours after injections, mice were sacrificed and organs removed for fluorescent quantification of protein tropism to tumors (* indicates p=0.014; n=6). FIG. 18B shows overlaid fluorescent images of tumors and muscle from Control and DMXAA-injected mice. Tumor-specific tropism of fibrinogen was observed over albumin. FIG. 18C shows overlaid fluorescence of tumors from mice injected with saline and colchicine, a small molecule vascular disrupting agent that binds to tubulin in tumor blood vessels. Mice bearing bilateral MDA-MB-435 tumors were intravenously injected with a mixture of fibrinogen and albumin that had been labeled with unique near-infrared fluorophores (VT750 and VT680, respectively). Immediately after injection, mice were intraperitoneally injected with colchicine (1 mg/kg) in 200 microliters of sodium phosphate buffer or vehicle. At 24 hours after injections, mice were sacrificed and organs removed for fluorescent quantification of protein tropism to tumors. Additionally, it was shown communication can be mediated by both short- and long-lived facets of coagulation (FXIII-activity and fibrin, respectively).

### Example 4

This example illustrates the induction of ICAM-1 expression in HUVECs. The cell surface glycoprotein Intercellular Cell Adhesion Molecule-1 (ICAM-1) is expressed at low levels in endothelial cells, but can be upregulated in response to certain growth factors and cytokines. Human Umbilical Vein Endothelial Cells (HUVECs) were cultured on slides coated with gelatin in Medium 200 supplemented with LSGS (Cascade Biologics/Invitrogen). One day after plating, cells were treated with 10 ng/ml TNF-alpha or vehicle alone overnight. The next day cells were fixed and immuno-stained for ICAM-1 expression (antibody from Cell Signaling). The results show an upregulation of ICAM-1 on the cell surface in cells treated with TNF-alpha. See FIG. 19. This experiment demonstrates a controlled induction of a cell surface molecule relevant to the inflammatory response in an endothelial cell type.

### Example 5

The complement cascade is an important pathway in the immune response to pathogens that results in the deposition of certain molecules on the cell surface. It also may be induced in response to the binding of exogenously-administered therapeutic antibodies on target cell surfaces. In this prophetic example, as a model interaction, sheep red blood cells (Innovative Research) will be sensitized to the complement cascade by incubation with anti-sheep red blood cell stroma from rabbit (Sigma-Aldrich). A control population of non-sensitized cells will also be included. The cells will then be incubated with sera containing the necessary complement cascade molecules (Quidel) to induce C3 deposition on sensitized cells. Cells will then be immuno-stained for the presence of C3 on the cell surface using an anti-C3 antibody (GenWay). The presence of C3 on only the sensitized cells will demonstrate a controlled deposition of a protein on the cell surface.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method, comprising:
administering, to a subject, a receiving composition comprising an agent able to bind a product of a biological cascade; and
prior to, simultaneously with, and/or after the act of administering, activating the biological cascade in the subject, thereby facilitating association of the receiving composition with the biological cascade.

2. The method of claim 1, wherein the biological cascade is a coagulation cascade.

3. The method of claim 1, wherein the biological cascade is a complement cascade.

4. The method of claim 1, wherein the cascade is an inflammation cascade.

5. (canceled)

6. The method of claim 1, wherein the agent is able to specifically bind the product of the biological cascade.

7.-8. (canceled)

9. The method of claim 1, wherein the product of the biological cascade that the agent binds catalyzes another reaction within the biological cascade.

10. The method of claim 1, wherein the product of the biological cascade that the agent binds is a protein.

11.-18. (canceled)

19. The method of claim 1, wherein the receiving composition further comprises a diagnostic agent.

20.-25. (canceled)

26. The method of claim 1, wherein the receiving composition further comprises a pharmaceutical agent.

27. The method of claim 26, wherein the pharmaceutical agent comprises an antitumor species.

28. (canceled)

29. The method of claim 1, wherein the receiving composition comprises a particle having an average dimension of less than about 1 micrometer.

30.-32. (canceled)

33. The method of any one of claim 1, comprising activating the biological cascade within a tumor.

34. The method of any one of claim 1, wherein the biological cascade is activated, at least in part, using an externally applied stimulus.

35. The method of claim 34, wherein the biological cascade is activated using heat.

36. The method of claim 34, wherein the biological cascade is activated using electromagnetic radiation.

37. (canceled)

38. The method of claim 1, wherein the biological cascade is activated using externally applied pressure.

39. The method of claim 1, wherein the biological cascade is activated by exposing the subject to an activation composition.

40. The method of claim 39, wherein the activation composition comprises a particle having an average dimension of less than about 1 micrometer.

41.-52. (canceled)

53. An article, comprising a receiving composition comprising an agent able to bind a product of a biological cascade, and an activation composition able to activate the biological cascade, wherein the receiving composition is distinguishable from the activation composition.

54.-68. (canceled)

69. A kit, comprising:
a receiving composition comprising an agent able to bind a product of a biological cascade; and
an activation composition able to activate the biological cascade.

70.-207. (canceled)


SEQUENCE LISTING (23 sequences)

SEQ ID NO: 1 - protein, 12 aa, Artificial Sequence
  Synthetic Peptide
GYXCXXGLCYIQ

SEQ ID NO: 2 - protein, 15 aa, Artificial Sequence
  Synthetic Peptide
GNQEQVSPLTLLKXC

SEQ ID NO: 3 - protein, 16 aa, Artificial Sequence
  Synthetic Peptide
XXXGYXCXXGLCYIQK

SEQ ID NO: 4 - protein, 2 aa, Artificial Sequence
  Synthetic Peptide
KK

SEQ ID NO: 5 - protein, 6 aa, Artificial Sequence
  Synthetic Peptide
HSSKLQ

SEQ ID NO: 6 - protein, 5 aa, Artificial Sequence
  Synthetic Peptide
PICFF

SEQ ID NO: 7 - protein, 7 aa, Artificial Sequence
  Synthetic Peptide
GPLGVRG

SEQ ID NO: 8 - protein, 10 aa, Artificial Sequence
  Synthetic Peptide
GVSQNYPIVG

SEQ ID NO: 9 - protein, 10 aa, Artificial Sequence
  Synthetic Peptide
LVLASSSFGY

SEQ ID NO: 10 - protein, 4 aa, Artificial Sequence
  Synthetic Peptide
DEVD

SEQ ID NO: 11 - protein, 4 aa, Artificial Sequence
  Synthetic Peptide
WEHD

SEQ ID NO: 12 - protein, 16 aa, Artificial Sequence
  Synthetic Peptide
GNAEQVSPLTLLKXCK

SEQ ID NO: 13 - protein, 12 aa, Artificial Sequence
  Synthetic Peptide
GLPCDYYGTCLD

SEQ ID NO: 14 - protein, 12 aa, Artificial Sequence
  Synthetic Peptide
GYLCGDYTLCPD

SEQ ID NO: 15 - protein, 10 aa, Artificial Sequence
  Synthetic Peptide
LPCDYYGTCX

SEQ ID NO: 16 - protein, 11 aa, Artificial Sequence
  Synthetic Peptide
LPCDYYGTCXX

SEQ ID NO: 17 - protein, 10 aa, Artificial Sequence
  Synthetic Peptide
CGLIIQKNEC

SEQ ID NO: 18 - protein, 10 aa, Artificial Sequence
  Synthetic Peptide
CNAGESSKNC

SEQ ID NO: 19 - protein, 8 aa, Artificial Sequence
  Synthetic Peptide
XCGRGDSP

SEQ ID NO: 20 - protein, 9 aa, Artificial Sequence
  Synthetic Peptide
XCGNGRAHA

SEQ ID NO: 21 - DNA, 45 bp, Artificial Sequence
  Synthetic Oligonucleotide
catgccatgggatcaggcactacaaatactgtggcagcatataat

SEQ ID NO: 22 - DNA, 43 bp, Artificial Sequence
  Synthetic Oligonucleotide
cgggatcctattatggagaatcacctcttcctctgaattcccc

SEQ ID NO: 23 - DNA, 46 bp, Artificial Sequence
  Synthetic Oligonucleotide
cgggatcctattatgcatgtgctcttccgttacctctgaattcccc